(19) United States
(12) Patent Application Publication
(10) Pub. No.: US 2009/0325878 A1
(43) Pub. Date: Dec. 31, 2009

(54) ORGAN PRECONDITIONING, ARREST, PROTECTION, PRESERVATION AND RECOVERY

(75) Inventor: Geoffrey Phillip Dobson, Wulguru (AU)

Correspondence Address: VENABLE LLP, P.O. BOX 34385, WASHINGTON, DC, 20043-9998, US

(73) Assignee: Hibernation Therapeutics Limited, Brisbane (AU)

(21) Appl. No.: 12/472,069

(22) Filed: May 26, 2009

Related U.S. Application Data
(63) Continuation of application No. 10/539,222, filed on Jun. 17, 2005, now abandoned.
(60) Provisional application No. 60/436,175, filed on Dec. 23, 2002.

(30) Foreign Application Priority Data
Jan. 23, 2003 (AU) 2003900296
Jun. 20, 2003 (AU) 2003903127

(51) Int. Cl. A61K 38/17; A61K 31/167; A61K 31/7076; A61K 38/08; A61K 31/517; A61K 31/135

(52) U.S. Cl. 514/12; 514/626; 514/46; 514/17; 514/252.17; 514/654

(57) ABSTRACT

The present invention relates to a method for reducing electrical disturbance of a cell's resting membrane potential comprising administering an effective amount of a composition comprising an effective amount of a local anaesthetic and of one or more of a potassium channel opener, an adenosine receptor agonist, an anti-adrenergic, a calcium antagonist, an opioid, an NO donor and a sodium hydrogen exchange inhibitor.

### FIELD OF THE INVENTION

The present invention relates to a composition for arresting, protecting or preserving a cell, tissue or organ, and uses of the composition for preconditioning, arresting, protecting or preserving a cell, tissue or organ, in particular the heart. The present invention also provides a method for arresting, protecting or preserving a cell, tissue or organ, in particular the heart during open-heart surgery, cardiovascular diagnosis or therapeutic intervention. The invention also relates to a method of recovering a cell, tissue or organ from arrest. The invention also provides a method for preconditioning and protecting a cell, tissue or organ from damage during therapeutic intervention and/or ischaemia.

### BACKGROUND OF THE INVENTION

Globally there are over 1 million elective open-heart surgery operations performed each year. One to three percent of these patients will die in the operating room, 10% of patients will leave with left ventricular dysfunction and 24% of high risk patients will die within 3 years. Moreover, in patients with elevated blood levels of creatine kinase (CK-MB) immediately following surgery, there is a significantly higher risk of early (first year) and late (3 to 5 years) mortality. Perioperative and post-operative mortality and morbidity are related to iatrogenic ischemia-reperfusion injury during cardiac surgery, and to inadequate myocardial protection.

In 2000, approximately 1.2 million open-heart surgeries were performed worldwide. About 64% of these were coronary artery bypass graft procedures, 24% were heart valve replacement or repair procedures, and about 12% were related to the repair of congenital heart defects¹. About 1.2% were neonatal. The majority of open heart surgery operations (over 80%) require cardiopulmonary bypass and elective heart arrest using either a blood or crystalloid cardioplegia solution. During these procedures the heart may be arrested for 3 hrs, and a maximum of 4 hrs. About 10% of patients undergoing open-heart surgery will have post-operative left-ventricular dysfunction, and up to 30% will have atrial fibrillation following surgery². 3-5% of patients die in the operating room and 24% of high risk patients die within 3 years following surgery³. The amount of damage to the heart caused by 3-4 hrs is such that the heart is increasingly less likely to recover function, and more likely than not recover after 4 hrs arrest.

Currently the majority of cardioplegia solutions used contain high potassium (in excess of 15-20 mM).⁴⁻⁶ These include the widely used St Thomas No. 2 Hospital Solution which generally contains 110 mM NaCl, 16 mM KCl, 16 mM MgCl₂, 1.2 mM CaCl₂ and 10 mM NaHCO₃ and has a pH of about 7.8⁷. High potassium solutions usually lead to membrane depolarisation from about −80 to −50 mV⁷. Notwithstanding hyperkalemic solutions providing acceptable clinical outcomes, recent evidence suggests that progressive potassium induced depolarisation leads to ionic and metabolic imbalances that may be linked to myocardial stunning, ventricular arrhythmias, ischaemic injury, endothelial cell swelling, microvascular damage, cell death and loss of pump function during the reperfusion period. Infant hearts are even more prone to damage with prolonged cardioplegic arrest from high potassium than adult hearts⁷⁻¹⁰.

The major ion imbalances postulated are linked to an increased sodium influx which in turn activates the Na⁺/Ca²⁺ exchangers leading to a rise in intracellular Ca²⁺¹¹. Compensatory activation of Na⁺ and Ca²⁺ ion pumps then occur, which activate anaerobic metabolism to replenish ATP with a concomitant increase in tissue lactate and fall in tissue pH⁷. Potentially damaging free radical generation and oxidative stress have also been implicated in high potassium arrest and partially reversed by the administration of antioxidants. In some cases, high potassium induced ischaemia has been reported to have damaged smooth muscle and endothelial function which can compromise coronary artery flow⁸.

In an attempt to minimise ischaemic injury during cardioplegic arrest, an increasing number of experimental studies have employed potassium channel openers instead of high potassium¹² Cardioprotection using nicorandil, aprikalim or pinacidil is believed to be linked to the opening of the potassium channel which leads to a hyperpolarised state, a shortening of the action potential and decreasing Ca²⁺ influx into the cell¹⁰. One shortfall however is that the heart takes the same time or longer to recover with no improvement in function than with high potassium cardioplegic solutions. Another limitation is that pinacidil requires a carrier due to its low solubility in aqueous solutions. The carrier routinely used is dimethyl sulphoxide (DMSO) which is controversial when used in animal or human therapy.

Most investigators, including those who advocate using potassium channel openers, believe that as soon as blood flow is halted and the arrest solution administered, ischaemia occurs and progressively increases with time. To reduce the likelihood of damage, the applicant sought a cardioplegic solution that would place the heart in a reversible hypometabolic state analogous to the tissues of a hibernating turtle, a hummingbird in torpor or an aestivating desert frog. When these animals drop their metabolic rate (some by over 90%), their tissues do not become progressively ischaemic but remain in a down-regulated steady state where supply and demand are matched. An ideal cardioplegic solution should produce a readily reversible, rapid electrochemical arrest with minimal tissue ischaemia. Ideally, the heart should accumulate low tissue lactate, utilise little glycogen, show minimal changes in high-energy phosphates, cytosolic redox (NAD/NADH) and the bioenergetic phosphorylation (ATP/ADP Pi) ratio and free energy of ATP. There should be little or no change in cytosolic pH or free magnesium, minimal water shifts between the intracellular and extracellular phases, and no major ultrastructural damage to organelles such as the mitochondria. The ideal cardioplegic solution should produce 100% functional recovery with no atrial fibrillation, ventricular arrhythmias, cytosolic calcium overload, or other pump abnormalities. There is no cardioplegic solution currently available which fulfils all these requirements.

Ischaemic heart disease is the single leading cause of death in the US and industrialized nations¹. Each year, about 1.1 million US people suffer a heart attack, and industry estimates there are over 2.7 million cases globally per annum. About 42% of heart attacks (ie 460,000 patients in the USA) are fatal, and half of these occur within the first hour of experiencing symptoms and before the patient reaches the hospital. Ischaemia (literally “to hold back blood”) is usually defined as an imbalance between blood supply and demand to an organ or tissue and results in deficient oxygen, fuel or nutrient supply to cells. The most common cause of ischaemia is a narrowing of the artery or, in the extreme case, from a blood clot blocking the artery. In 90% of cases a blood clot is usually formed from rupture of an atherosclerotic plaque.

The response of a cell to ischaemia depends upon the time and extent of the deprivation of blood supply. A large percentage of deaths from cardiac ischaemia are due to ventricular fibrillation (VF) associated with profound metabolic, ionic and functional disturbances. Within seconds to minutes of coronary artery occlusion there is a shift from aerobic to anaerobic metabolism, a decrease in high-energy phosphates (phosphocreatine, ATP), glycogen loss, lactate accumulation, tissue acidosis, a rise in intracellular Na⁺ and Ca²⁺ and extracellular K⁺ as well as changes to the transmembrane potential and ventricular dysfunction. Restoration of coronary flow within 15 min can lead to full recovery of the heart^{13,14}. However, it can also stun the myocardium leading to potentially fatal arrhythmias¹⁵. If ischaemia persists beyond 15 min, the deprived area of the heart will undergo a progressive loss of ATP, increased Na⁺ and Ca²⁺ entry, severe membrane injury, mitochondrial dysfunction, and the closing of gap junctions between cells thereby electrically isolating the damaged cells and eventually, cell death will occur¹⁶.

While early reperfusion, or restoration of the blood flow, remains the most effective means of salvaging the myocardium from acute ischaemia, the sudden influx of oxygen paradoxically may lead to further necrosis, ventricular arrhythmias and death¹⁶⁻¹⁹. The extent of “reperfusion injury” has been linked to a cascade of inflammatory reactions including the generation of cytokines, leukocytes, reactive oxygen species and free radicals²⁰.

Reperfusion of ischaemic myocardium is necessary to salvage tissue from eventual death^{22,28}. However, reperfusion after even brief periods of ischaemia is associated with pathologic changes that represent either an acceleration of processes initiated during ischaemia per se, or new pathophysiological changes that were initiated after reperfusion. The degree and extent of reperfusion injury can be influenced by inflammatory responses in the myocardium. Ischaemia-reperfusion prompts a release of oxygen free radicals, cytokines and other pro-inflammatory mediators that activate both the neutrophils and the coronary vascular endothelium. The inflammatory process can lead to endothelial dysfunction, microvascular collapse and blood flow defects, myocardial infarction and apoptosis²². Pharmacologic anti-inflammatory therapies targeting specific steps have been shown to decrease infarct size and myocardial injury. Adenosine and nitric oxide are two compounds which have been observed to have beneficial effects against such neutrophil-mediated inflammation.

In 1990, Homeister and colleagues aimed to limit reperfusion injury by administering an intravenous bolus of lidocaine (2 mg/kg) in open-chest dogs 1 min before a 90 min occlusion of the left circumflex coronary artery and again 1 min before reperfusion²⁹. At reperfusion, adenosine was infused (150 μg/kg/ml/min) through an intracoronary catheter and continued for 1-hour reperfusion. It was concluded that the sequential treatment of lidocaine and adenosine reduced infarct size²⁹. In 1996, Vander-Heide and Reimer³⁰ failed to reproduce these findings in the same model and concluded that intravenous adenosine therapy (150 μg/kg/ml/min) during reperfusion with or without lidocaine pretreatment did not limit infarct size after 90 min regional ischaemia. In an attempt to clarify the issue, Garratt³¹ and Mahaffey,³² administered lidocaine and adenosine sequentially and separately in humans during balloon angioplasty and thrombolytic therapy respectively, but the results were again conflicting. Garratt and colleagues³¹ proposed a potential benefit in 35 patients whereas Mahaffey and colleagues, in the larger AMISTAD trials involving 236 acute myocardial infarction patients, concluded that the presence of lidocaine made no difference to the outcome of adenosine-treated patients in reducing infarct size. Indeed, the clinical outcomes of the adenosine-treated group in the AMISTAD trials tended to be slightly worse than in the placebo group³².

The applicant previously found that the heart can be better protected by using a potassium channel opener and/or an adenosine receptor agonist (preferably adenosine) and a local anaesthetic (preferably lidocaine or lignocaine) to arrest and then preserve the heart under physiological concentrations of potassium. Thus reducing the risk of potassium induced injury to the tissue which prior art high potassium arrest solutions may induce (see WO 00/56145)^{33,34} (the entire disclosure of which is incorporated herein by reference). In this reference these components are administered in a single preparation or simultaneously.

This cardioplegia solution containing the combination of the potassium channel opener and local anaesthetic was shown by the applicant to generally improve functional recovery from arrest of the organ over existing solutions.

This solution provides improved functional recovery of the arrested heart. However, functional recovery is still decreased with increasing arrest time. Accordingly, there is a need for a method which further improves functional recovery of an arrested tissue, and/or reduces damage to an arrested tissue, and more particularly after increasing arrest time of the tissue. In particular, there is a need for improved protection of the tissue from damage during arrest.

Also, as stated above, this solution results in the arrest of the heart under physiological potassium concentrations. The arrested heart is then reperfused (ie, blood flow restored) to recover function. However, there are also risks in further damaging the heart at reperfusion. Accordingly, there is also a need for a method of recovering a tissue from arrest, with improved functional recovery during reperfusion.

The heart possesses an extraordinary ability to ‘remember’ short episodes of sublethal ischaemia-reperfusion (angina) which protects the myocardium and microvascular from a subsequent lethal period of ischaemia (infarction)^{41,42}. The phenomenon, known as “ischaemic preconditioning” or “preconditioning”, is the most powerful means of delaying cell death known. It was first described in 1986 by Murry, Jennings and Reimer who reported an infarct size reduction from 29% to 7% in anaesthetised open-chested dogs after three brief episodes of brief ischaemia followed by 40 min coronary artery occlusion⁴¹. Since that time, the phenomenon has been described in tissues and organs of most animal models studied⁴³, including human^{44,45}. Two different time frames for preconditioning have been identified; an early “classical” window that lasts 1 to 3 hrs after the stimulus, and a later “delayed” window which develops over many hours and can last up to 12 to 72 hours^{18,36,43,46}. The heart can also be protected by preconditioning other organs such as kidney or intestine. This phenomenon is termed “remote preconditioning”⁴⁷.

However, most clinicians are reluctant to precondition a patient's diseased heart by temporarily tying off the vessel in the clinical setting⁴⁵. Therefore, there is also a need to develop a pharmacological mimetic or composition for preconditioning tissue, to protect the tissue from a subsequent period of ischemia without the need to physically tie-off vessels.

### SUMMARY OF THE INVENTION

This invention is directed towards overcoming, or at least alleviating, one or more of the difficulties or deficiencies associated with the prior art.

In one embodiment, the invention provides a method for reducing electrical disturbance of a cell's resting membrane potential comprising administering an effective amount of a composition comprising an effective amount of a local anaesthetic and of one or more of a potassium channel opener, an adenosine receptor agonist, an anti-adrenergic, a calcium antagonist, an opioid, an NO donor and a sodium hydrogen exchange inhibitor.

In another embodiment, the invention provides a method for reducing damage to an cell, tissue or organ following ischaemia comprising administering an effective amount of a composition comprising an effective amount of a local anaesthetic and of one or more of a potassium channel opener, an adenosine receptor agonist, an anti-adrenergic, a calcium antagonist, an opioid, an NO donor and a sodium hydrogen exchange inhibitor.

In another embodiment, the invention provides a method for preconditioning a cell or tissue during ischaemia or reperfusion comprising administering an effective amount of a composition comprising an effective amount of a local anaesthetic and of one or more of a potassium channel opener, an adenosine receptor agonist, an anti-adrenergic, a calcium antagonist, an opioid, an NO donor and a sodium hydrogen exchange inhibitor.

In another embodiment, the invention provides a method for reducing damage to cells, organs and tissues before, during and following a surgical or clinical intervention comprising administering an effective amount of a composition comprising an effective amount of a local anaesthetic and of one or more of a potassium channel opener, an adenosine receptor agonist, an anti-adrenergic, a calcium antagonist, an opioid, an NO donor and a sodium hydrogen exchange inhibitor.

In another embodiment, the invention provides a method for reducing either or both inflammation and clotting in a tissue or organ comprising administering an effective amount of a composition comprising an effective amount of a local anaesthetic and of one or more of a potassium channel opener, an adenosine receptor agonist, an anti-adrenergic, a calcium antagonist, an opioid, an NO donor, a protease inhibitor and a sodium hydrogen exchange inhibitor.

The methods of the invention are applicable to any cell, tissue or organ. Examples include where the cell is a myocyte, endothelial cell, smooth-muscle cell, neutrophil, platelet and other inflammatory cells, or the tissue is heart tissue or vasculature, or the organ is a heart.

In some embodiments, the composition used in these methods further comprises one or more of an antioxidant, ionic magnesium, an impermeant and a metabolic substrate. The composition may also oxygenated. The composition may also be formulated into a medicament by combining with a blood-based or crystalloid (non-cell, non-protein) carrier. In such a medicament, it is desirable in some applications to that the concentrations of one or more of sodium, calcium and chloride are lower than physiological concentrations. Also, it is desirable to use the medicaments at different temperatures, namely: profound hypothermia (0 to 4 degrees Celsius), moderate hypothermia (5 to 20 degrees Celsius), mild hypothermia (20 to 32 degrees Celsius) or normothermia (32 to 38 degrees Celsius).

The components of the medicament or composition may be combined before administration or when the components are administered substantially simultaneously or co-administered.

### DETAILED DESCRIPTION

The applicant has surprisingly found that the simultaneous delivery of a solution a local anaesthetic together with component(s) as detailed below prior to, during or following ischaemia markedly reduces cell damage resulting from ischaemia. In particular, continuous administration of a solution (which may be carried in physiological saline or compatible fluid (eg, patient's own blood)) of the components results in significantly less damage to a cell, organ or tissue, such as a heart, than delivery of the components of the composition independently (eg, one component (adenosine) parenterally and the other (lignocaine) in intermittent bolus doses).

The simultaneous delivery of the two components briefly prior to ischaemia, throughout ischaemia and reperfusion shows surprising increased efficacy. In another aspect of the invention, there is provided a method of reducing myocardial tissue damage during a heart attack or cardioplegia by delivering the composition to the tissue. In another aspect of the invention, there is provided a method of protecting myocardial tissue from reperfusion injury, including inflammatory and blood coagulation effects often experienced during reperfusion following an ischaemic event.

The invention also provides a method for reducing infarction size and/or reducing inflammation and blood coagulation responses in myocardial tissue during ischaemia and/or reperfusion.

The invention also provides a method for reducing electrical disturbances in the heart such as atrial or ventricular arrhythmias (including lethal ventricular tachycardias and ventricular fibrillation) during ischaemia and/or reperfusion.

The composition of the present invention protects the organ after arrest of the organ, with good to excellent recoveries of function after reperfusion.

The invention also provides a use of the composition (especially the preferred embodiments described below) in the methods described above. This use of the composition can extend to many therapeutic applications, including without limitation, cardiovascular diagnosis (including coronary angiography, myocardial scintigraphy, non-invasive diagnosis of dual AV nodal conduction), use in treatment of heart attack, resuscitation therapy, short-term and long-term storage of organs tissues or cells (including graft vessels), use before, prior to, during or following open-heart surgery, angioplasty and other therapeutic interventions.

In one embodiment, the composition comprises adenosine and lignocaine. In particular, the composition may include adenosine and lignocaine in the weight ratio of about 1:2.

In this application, without being bound by this mode of action, protection is thought to involve a multi-tiered system from modulating membrane excitability to a multitude of intracellular signaling pathways leading to (i) reduced ion imbalances, in particular sodium and calcium ion loading in the cells, (ii) improved atrial and ventricular matching of electrical conduction to metabolic demand, which may involve modulation of gap junction communication, (iii) vasodilation of coronary arteries and (ii) attenuation of the inflammatory response to injury

Infusion of the composition during pretreatment and ischaemia and reperfusion provides continuous protection from ischaemic tissue injury including protection from lethal arrhythmias. The protection from localised injury and inflammation can also be obtained when placing a stent into a vessel such as during angioplasty. The composition is also used within a polymer or special coating for a stent for use in any vessel of the body including coronary arteries, carotid arteries, or leg arteries of the body.

The composition according to the invention includes a potassium channel opener. Potassium channel openers are agents which act on potassium channels to open them through a gating mechanism. This results in efflux of potassium across the membrane along its electrochemical gradient which is usually from inside to outside of the cell. Thus potassium channels are targets for the actions of transmitters, hormones, or drugs that modulate cellular function. It will be appreciated that the potassium channel openers include the potassium channel agonists which also stimulate the activity of the potassium channel with the same result. It will also be appreciated that there are diverse classes of compounds which open or modulate different potassium channels; for example, some channels are voltage dependent, some rectifier potassium channels are sensitive to ATP depletion, adenosine and opioids, others are activated by fatty acids, and other channels are modulated by ions such as sodium and calcium (ie. channels which respond to changes in cellular sodium and calcium). More recently, two pore potassium channels have been discovered and thought to function as background channels involved in the modulation of the resting membrane potential.

Potassium channel openers may be selected from the group consisting of: nicorandil, diazoxide, minoxidil, pinacidil, aprikalim, cromokulim and derivative U-89232, P-1075 (a selective plasma membrane KATP channel opener), emakalim, YM-934, (+)-7,8-dihydro-6,6-dimethyl-7-hydroxy-8-(2-oxo-1-piperidinyl)-6H-pyrano[2,3-f]benz-2,1,3-oxadiazole (NIP-121), RO316930, RWJ29009, SDZPCO400, rimakalim, symakalim, YM099, 2-(7,8-dihydro-6,6-dimethyl-6H-[1,4]oxazino[2,3-f][2,1,3]benzoxadiazol-8-yl) pyridine N-oxide, 9-(3-cyanophenyl)-3,4,6,7,9,10-hexahydro-1,8-(2H,5H)-acridinedione (ZM244085), [(9R)-9-(4-fluoro-3-125iodophenyl)-2,3,5,9-tetrahydro-4H-pyrano[3,4-b]thieno[2,3-e]pyridin-8(7H)-one-1,1-dioxide] ([125I]A-312110), (−)-N-(2-ethoxyphenyl)-N′-(1,2,3-trimethylpropyl)-2-nitroethene-1,1-diamine (Bay X 9228), N-(4-benzoyl phenyl)-3,3,3-trifluoro-2-hydroxy-2-methylpropionamine (ZD6169), ZD6169 (KATP opener) and ZD0947 (KATP opener), WAY-133537 and a novel dihydropyridine potassium channel opener, A-278637. In addition, potassium channel openers can be selected from BK-activators (also called BK-openers or BK(Ca)-type potassium channel openers or large-conductance calcium-activated potassium channel openers) such as benzimidazolone derivatives NS004 (5-trifluoromethyl-1-(5-chloro-2-hydroxyphenyl)-1,3-dihydro-2H-benzimidazole-2-one), NS1619 (1,3-dihydro-1-[2-hydroxy-5-(trifluoromethyl)phenyl]-5-(trifluoromethyl)-2H-benzimidazol-2-one), NS1608 (N-(3-(trifluoromethyl)phenyl)-N′-(2-hydroxy-5-chlorophenyl)urea), BMS-204352, retigabine (also GABA agonist). There are also intermediate (eg. benzoxazoles, chlorzoxazone and zoxazolamine) and small-conductance calcium-activated potassium channel openers.

In addition, potassium channel openers may act as indirect calcium antagonists, ie they act to reduce calcium entry into the cell by shortening the cardiac action potential duration through the acceleration of phase 3 repolarisation, and thus shorten the plateau phase. Reduced calcium entry is thought to involve L-type calcium channels, but other calcium channels may also be involved.

Some embodiments of the invention utilise direct calcium antagonists, the principal action of which is to reduce calcium entry into the cell. These are selected from at least five major classes of calcium channel blockers as explained in more detail below. It will be appreciated that these calcium antagonists share some effects with potassium channel openers, particularly ATP-sensitive potassium channel openers, by inhibiting calcium entry into the cell.

Adenosine is particularly preferred as the potassium channel opener or agonist. Adenosine is capable of opening the potassium channel, hyperpolarising the cell, depressing metabolic function, possibly protecting endothelial cells, enhancing preconditioning of tissue and protecting from ischaemia or damage. Adenosine's actions are complex as the drug has many broad-spectrum properties. Adenosine has been shown to increase coronary blood flow³⁵, hyperpolarise the cell membrane, and protect during ischemia and reperfusion²². Adenosine also acts as a ‘early’ and ‘delayed’ preconditioning ‘trigger’ or agent to protect the heart against ischaemic injury^{36,37}. Part of adenosine's cardioprotective properties are believed to be activation of one or more of the adenosine receptor subtypes (A1, A2a, A2b and A3)³⁸. Much of adenosine's protection has been ascribed to A1 and A3 receptor activation and their associated transduction pathways leading to preconditioning, protection and preservation of cell integrity³⁹. It is also known that adenosine, by activating A1 receptors, is involved in slowing the sinoatrial nodal pacemaker rate (negative chronotropy), delaying atrioventricular (A-V) nodal impulse conduction (negative dromotropy), reduces atrial contractility (negative inotropy), and inhibits the effect of catecholamines (anti-adrenergic effect)⁴⁰. The A1-stimulated negative chronotropic, dromotropic and inotropic effects of adenosine are linked to the drug's action to reduce the activity of adenyl cyclase, to activate the inward rectifier potassium current (I_{K-Ado}), inhibition of phospholipid turnover, activation of ATP-sensitive K channels, inhibits effect of catecholamines on the L-type Ca²⁺ current and activation of nitric oxide synthase in AV nodal cells. A3 receptors have also shown to have direct cardioprotective effects, and A2 receptors have potent vasodilatory and anti-inflammatory actions in response to injury^{22,38}. Adenosine is also an indirect calcium antagonist, vasodilator, antiarrhythmic, antiadrenergic, free radical scavenger, arresting agent, anti-inflammatory agent (attenuates neutrophil activation), analgesic, metabolic agent and possible nitric oxide donor.

It will be appreciated that anti-adrenergics such as beta-blockers, for example, esmolol, atenolol, metoprolol and propranolol could be used instead of or in combination with the potassium channel opener to reduce calcium entry into the cell. Preferably, the beta-blocker is esmolol. Similarly, alpha(1)-adrenoceptor-antagonists such as prazosin, could be used instead of or in combination with the potassium channel opener to reduce calcium entry into the cell and therefore calcium loading.

In one aspect of the invention there is provided a method for preconditioning, arresting, protecting and/or reducing damage to tissues during ischemia or reperfusion comprising delivery of an effective amount of:

an antiadrenergic; and

a local anaesthetic.

According to this aspect of the present invention there is also provided a composition including an effective amount of an antiadrenergic and a local anaesthetic.

Preferably, the antiadrenergic is a beta-blocker. Preferably the beta-blocker is esmolol.

Adenosine is also known to indirectly inhibit the sodium-calcium exchanger which would reduce cell sodium and calcium loading. It will be appreciated that inhibitors of the sodium-calcium exchanger would lead to reduced calcium entry and magnify the effect of adenosine. Na⁺/Ca²⁺ exchange inhibitors may include benzamyl, KB-R7943 (2-[4-(4-Nitrobenzyloxy)phenyl]ethyl]isothiourea mesylate) or SEA0400 (2-[4-[(2,5-difluorophenyl)methoxy]phenoxy]-5-ethoxyaniline).

Since one of adenosine's properties is to reduce calcium entry and sodium entry in the heart and coronary vascular cells, it will be further appreciated that a compound leading to reduced calcium and sodium entry (or reduce calcium oscillations in the cell) before, during and/or following treatment could be used instead of or in combination with adenosine to reduce calcium entry into the cell. Such compounds may be selected from calcium channel blockers from three different classes: 1,4-dihydropyridines (eg. nitrendipine), phenylalkylamines (eg. verapamil), and the benzothiazepines (e.g. diltiazem, nifedipine).

Calcium channel blockers are also called calcium antagonists or calcium blockers. They are often used clinically to decrease heart rate and contractility and relax blood vessels. They may be used to treat high blood pressure, angina or discomfort caused by ischaemia and some arrhythmias, and they share many effects with beta-blockers (see discussion above).

Five major classes of calcium channel blockers are known with diverse chemical structures: 1. Benzothiazepines: eg Diltiazem, 2. Dihydropyridines: eg nifedipine, Nicardipine, nimodipine and many others, 3. Phenylalkylamines: eg Verapamil, 4. Diarylaminopropylamine ethers: eg Bepridil, 5. Benzimidazole-substituted tetralines: eg Mibefradil.

The traditional calcium channel blockers bind to L-type calcium channels (“slow channels”) which are abundant in cardiac and smooth muscle which helps explain why these drugs have selective effects on the cardiovascular system. Different classes of L-type calcium channel blockers bind to different sites on the alpha1-subunit, the major channel-forming subunit (alpha2, beta, gamma, delta subunits are also present). Different sub-classes of L-type channel are present which may contribute to tissue selectivity. More recently, novel calcium channel blockers with different specificities have also been developed for example, Bepridil, is a drug with Na+ and K+ channel blocking activities in addition to L-type calcium channel blocking activities. Another example is Mibefradil, which has T-type calcium channel blocking activity as well as L-type calcium channel blocking activity.

Three common calcium channel blockers are diltiazem (Cardizem), verapamil (Calan) and Nifedipine (Procardia). Nifedipine and related dihydropyridines do not have significant direct effects on the atrioventricular conduction system or sinoatrial node at normal doses, and therefore do not have direct effects on conduction or automaticity. While other calcium channel blockers do have negative chronotropic/dromotropic effects (pacemaker activity/conduction velocity). For example, Verapamil (and to a lesser extent diltiazem) decreases the rate of recovery of the slow channel in AV conduction system and SA node, and therefore act directly to depress SA node pacemaker activity and slow conduction. These two drugs are frequency- and voltage-dependent, making them more effective in cells that are rapidly depolarizing. Verapamil is also contraindicated in combination with beta-blockers due to the possibility of AV block or severe depression of ventricular function. In addition, mibefradil has negative chronotropic and dromotropic effects. Calcium channel blockers (especially verapamil) may also be particularly effective in treating unstable angina if underlying mechanism involves vasospasm.

Omega conotoxin MVIIA (SNX-111) is an N type calcium channel blocker and is reported to be 100-1000 fold more potent than morphine as an analgesic but is not addictive. This conotoxin is being investigated to treat intractible pain. SNX-482 a further toxin from the venom of a carnivorous spider venom, blocks R-type calcium channels. The compound is isolated from the venom of the African tarantula, Hysterocrates gigas, and is the first R-type calcium channel blocker described. The R-type calcium channel is believed to play a role in the body's natural communication network where it contributes to the regulation of brain function. Other Calcium channel blockers from animal kingdom include Kurtoxin from South African Scorpion, SNX-482 from African Tarantula, Taicatoxin from the Australian Taipan snake, Agatoxin from the Funnel Web Spider, Atracotoxin from the Blue Mountains Funnel Web Spider, Conotoxin from the Marine Snail, HWTX-I from the Chinese bird spider, Grammotoxin SIA from the South American Rose Tarantula. This list also includes derivatives of these toxins that have a calcium antagonistic effect.

Direct ATP-sensitive potassium channel openers (eg nicorandil, aprikalem) or indirect ATP-sensitive potassium channel openers (eg adenosine, opioids) are also indirect calcium antagonists and reduce calcium entry into the tissue. One mechanism believed for ATP-sensitive potassium channel openers also acting as calcium antagonists is shortening of the cardiac action potential duration by accelerating phase 3 repolarisation and thus shortening the plateau phase. During the plateau phase the net influx of calcium may be balanced by the efflux of potassium through potassium channels. The enhanced phase 3 repolarisation may inhibit calcium entry into the cell by blocking or inhibiting L-type calcium channels and prevent calcium (and sodium) overload in the tissue cell.

Potential targets for the combinational therapy include cardioplegia, management of ischaemic syndromes without or without clot-busters, cardiac surgery (on and off-pump), arrhythmia management, coronary interventions (balloon and stent), preconditioning an organ, tissue or cell to ischaemic stress, longer-term organ or cell preservation, peri- and post-operative pain management, peri- and post operative anti-inflammatory treatments, peri- and post operative anti-clotting strategies, resuscitation therapies, and other related therapeutic interventions.

Calcium channel blockers can be selected from nifedipine, nicardipine, nimodipine, nisoldipine, lercanidipine, telodipine, angizem, altiazem, bepridil, amlodipine, felodipine, isradipine and cavero and other racemic variations. In addition, it will be appreciated that calcium entry could be inhibited by other calcium blockers which could be used instead of or in combination with adenosine and include a number of venoms from marine or terrestrial animals such as the omega-conotoxin GVIA (from the snail conus geographus) which selectively blocks the N-type calcium channel or omega-agatoxin IIIA and IVA from the funnel web spider Agelelnopsis aperta which selectively blocks R- and P/Q-type calcium channels respectively. There are also mixed voltage-gated calcium and sodium channel blockers such as NS-7. to reduce calcium and sodium entry and thereby assist cardioprotection.

It will be appreciated that a calcium channel blocker could be used instead of or in combination with the a local anaesthetic.

Thus, in another aspect of the invention there is provided a method for preconditioning, arresting, protecting and/or reducing damage to a tissue during ischemia or reperfusion comprising delivery of an effective amount of:

a calcium channel blocker; and

potassium channel opener or adenosine receptor agonist.

According to this aspect of the invention there is also provided a composition including an effective amount of a calcium channel blocker and a local anaesthetic.

Preferably the calcium channel blocker is nifedipine.

In another embodiment, the composition according to the invention further includes an additional potassium channel opener. Preferably the additional potassium channel opener is diazoxide. Diazoxide is believed to preserve ion and volume regulation, oxidative phosphorylation and mitochondrial membrane integrity (appears concentration dependent). Diazoxide also affords cardioprotection by reducing mitochondrial oxidant stress at reoxygenation⁸¹. There is also some evidence that the protective effects of potassium channel openers are associated with modulation of reactive oxygen species generation in mitochondria^{42,49}.

The composition according to the invention includes an adenosine receptor agonist. It will be appreciated that the adenosine receptor agonists include compounds which act both directly and indirectly on the receptor resulting in activation of the receptor, or mimic the action of the receptor having the same net effect.

Suitable adenosine receptor agonists can be found in the reviews by Linden and colleagues^{38,72}, Hayes⁷² and Belardinelli⁷³. They may be selected from: N⁶-cyclopentyladenosine (CPA), N-ethylcarboxamido adenosine (NECA), 2-[p-(2-carboxyethyl)phenethyl-amino-5′-N-ethylcarboxamido adenosine (CGS-21680), 2-chloroadenosine, N⁶-[2-(3,5-demethoxyphenyl)-2-(2-methoxyphenyl]ethyladenosine, 2-chloro-N⁶-cyclopentyladenosine (CCPA), N-(4-aminobenzyl)-9-[5-(methylcarbonyl)-beta-D-robofuranosyl]-adenine (AB-MECA), ([IS-[1a,2b,3b,4a(S*)]]-4-[7-[[2-(3-chloro-2-thienyl)-1-methyl-propyl]amino]-3H-imidazole[4,5-b]pyridyl-3-yl]cyclopentane carboxamide (AMP579), N⁶—(R)-phenylisopropyladenosine (R-PLA), aminophenylethyladenosine 9APNEA) and cyclohexyladenosine (CHA)⁷². Others include full adenosine A1 receptor agonists such as N-[3-(R)-tetrahydrofuranyl]-6-aminopurine riboside (CVT-510), or partial agonists such as CVT-2759 and allosteric enhancers such as PD81723⁷⁴⁻⁷⁶. Other agonists include N6-cyclopentyl-2-(3-phenylaminocarbonyltriazene-1-yl)adenosine (TCPA), a very selective agonist with high affinity for the human adenosine A1 receptor⁷⁷, and allosteric enhancers of A1 adenosine receptor includes the 2-amino-3-naphthoylthiophenes⁷⁸.

CCPA is a particularly preferred adenosine receptor agonist. CCPA an A1 adenosine receptor agonist.

Thus, in another aspect, the invention provides a method for preconditioning, arresting, protecting and/or reducing damage to a tissue during ischemia or reperfusion comprising an effective amount of:

potassium channel opener or adenosine receptor agonist;

local anaesthetic; and

CCPA.

Modulation of agonist responses at the A1 adenosine receptor can also be achieved indirectly by an irreversible antagonist, receptor-G protein uncoupling and by the G protein activation state⁷⁹. Thus any agonist or antagonist which modulates the G protein activation state may be used to mimic adenosine receptor activation. There is also some evidence that there is some cross-talk between adenosine receptors. Furthermore, there is data suggesting that there are converging pathways and/or receptor cross-talk between adenosine 1 (and perhaps A3) receptors and deltal-opioid receptor mediated cardioprotection⁸⁰. Thus opioid receptor activation may result in identical protection as A1 receptor activation. It would be appreciated that Opioids could be used instead of or in combination with a potassium channel opener or adenosine receptor agonists.

Opioids, also known or referred to as opioid agonists, are a group of drugs that inhibit opium (Gr opion, poppy juice) or morphine-like properties and are generally used clinically as moderate to strong analgesics, in particular, to manage pain, both peri- and post-operatively. Other pharmacological effects of opioids include drowsiness, respiratory depression, changes in mood and mental clouding without loss of consciousness.

Opioids are also believed to be involved as part of the ‘trigger’ in the process of hibernation, a form of dormancy characterised by a fall in normal metabolic rate and normal core body temperature. In this hibernating state, tissues are better preserved against damage that may otherwise be caused by diminished oxygen or metabolic fuel supply, and also protected from ischemia reperfusion injury.

There are three types of opioid peptides: enkephalin, endorphin and dynorphin.

Opioids act as agonists, interacting with stereospecific and saturable binding sites, in the heart, brain and other tissues. Three main opioid receptors have been identified and cloned, namely mu, kappa, and delta receptors. All three receptors have consequently been classed in the G-protein coupled receptors family (which class includes adenosine and bradykinin receptors). Opioid receptors are further subtyped, for example, the delta receptor has two subtypes, delta-1 and delta-2.

Cardiovascular effects of opioids are directed within the intact body both centrally (ie, at the cardiovascular and respiratory centres of the hypothalamus and brainstem) and peripherally (ie, heart myocytes and both direct and indirect effects on the vasculature). For example, opioids have been shown to be involved in vasodilation. Some of the action of opioids on the heart and cardiovascular system may involve direct opioid receptor mediated actions or indirect, dose dependent non-opioid receptor mediated actions, such as ion channel blockade which has been observed with antiarrhythmic actions of opioids, such as arylacetamide drugs. It is also known that the heart is capable of synthesising or producing the three types of opioid peptides, namely, enkephalin, endorphin and dynorphin. However, only the delta and kappa opioid receptors have been identified on ventricular myocytes.

Without being bound by any mode of action, opioids are considered to provide cardioprotective effects, by limiting ischaemic damage and reducing the incidence of arrhythmias, which are produced to counter-act high levels of damaging agents or compounds naturally released during ischemia. This may be mediated via the activation of ATP sensitive potassium channels in the sarcolemma and in the mitochondrial membrane and involved in the opening potassium channels. Further, it is also believed that the cardioprotective effects of opioids are mediated via the activation of ATP sensitive potassium channels in the sarcolemma and in the mitochondrial membrane. Thus it is believed that the opioid can be used in stead or in combination with the potassium channel opener or adenosine receptor agonist as they are also involved in indirectly opening potassium channels.

It will be appreciated that the opioids include compounds which act both directly and indirectly on opioid receptors. Opioids also include indirect dose dependent, non-opioid receptor mediated actions such as ion channel blockade which have been observed with the antiarrhythmic actions of opioids.

Thus, in another aspect of the invention there is provided a method for preconditioning, arresting, protecting and/or reducing damage to an organ, tissue or cell during ischemia and/or reperfusion comprising delivery of an effective amount of:

an opioid; and

a local anaesthetic.

According to this aspect of the invention there is also provided a composition including an effective amount of opioid and a local anaesthetic.

Preferably the opioid is selected from enkephalins, endorphins and dynorphins.

Preferably, the opioid is an enkephalin which targets delta, kappa and/or mu receptors.

More preferably the opioid is selected from delta-1-opioid receptor agonists and delta-2-opioid receptor agonists.

D-Pen2, 5]enkephalin (DPDPE) is a particularly preferred Delta-1-Opioid receptor agonist.

Local anaesthetic agents are drugs which are used to produce reversible loss of sensation in an area of the body. Many local anaesthetic agents consist of an aromatic ring linked by a carbonyl containing moiety through a carbon chain to a substituted amino group. In general there are 2 classes of local anaesthetics defined by their carbonyl-containing linkage group. The ester agents include cocaine, amethocaine, procaine and chloroprocaine, whereas the amides include prilocalne, mepivacaine, bupivacaine, mexiletine and lignocaine. At high concentrations, many drugs that are used for other purposes possess local anaesthetic properties. These include opioid analgesics, Beta-adrenoceptor antagonists, anticonvulsants (lamotrigine and lifarizine) and antihistamines. The local anaesthetic component of the composition according to the present invention may be selected from these classes, or derivatives thereof, or from drugs than may be used for other purposes. Preferably, the component possesses local anaesthetic properties also.

Preferably the local anaesthetic is Lignocaine. In this specification Lignociane and Lidocaine are used interchangeably. Lignocaine is preferred as it is capable of acting as a local anaesthetic probably by blocking sodium fast channels, depressing metabolic function, lowering free cytosolic calcium, protecting against enzyme release from cells, possibly protecting endothelial cells and protecting against myofilament damage. At lower therapeutic concentrations lidocaine normally has little effect on atrial tissue, and therefore is ineffective in treating atrial fibrillation, atrial flutter, and supraventricular tachycardias⁶⁵. Lignocaine is also a free radical scavenger, an antiarrhythmic and has anti-inflammatory and anti-hypercoagulable properties. It must also be appreciated that at non-anaesthetic therapeutic concentrations, local anaesthetics like lidocaine may not completely block the voltage-dependent sodium fast channels, but down-regulate channel activity and reduce sodium entry^{82,83}. As an anti-arrhythmic, lidocaine is believed to target small sodium currents that normally continue through phase 2 of the action potential and consequently shortens the action potential and the refractory period⁶⁵.

Lignocaine is a local anaesthetic which is believed to block sodium fast channels and has anti-arrhythmatic properties by reducing the magnitude of inward sodium current⁶²⁻⁶⁵. In this specification, the terms “lidocaine” and “lignocaine” are used interchangeably. The accompanying shortening of the action potential is thought to directly reduce calcium entry into the cell via Ca²⁺ selective channels and Na⁺/Ca²⁺ exchange⁶⁵. Recent reports also implicate lignocaine with the scavenging of free radicals such as hydroxyl and singlet oxygen in the heart during reperfusion⁶⁶. Associated with this scavenging function, lignocaine may also inhibit phospholipase activity and minimise membrane degradation during ischaemia. Lignocaine can also depress vascular relaxations by a complex mechanism including poly(ADP-ribose) synthetase enzyme activity, but this effect has recently been shown to be pH dependent with little inhibition occurring below pH 7.2. Lignocaine's vasodilatory effects are believed due to calcium entry blockade that do not appear to involve Na⁺ channel blockade or opening of K⁺-channels⁶⁷. Lignocaine has also been shown to have a myocardial protective effect and in one study was found to be superior to high potassium solutions. However, these experiments show that lignocaine alone at 0.5, 1.0 and 1.5 mM gave highly variable functional recoveries using the isolated working rat heart. Lignocaine has also been shown to reduce infarct size in the brain and protect against reperfusion injury in the heart. More recently lignocaine has been shown to exhibit a number of pharmacological actions not related to the sodium channel block. For example, recent work has shown that local anaesthetics, including lignocaine, inhibit inflammatory responses^{68,69}. They also have beneficial effects in a number of pathological processes dependent on an overly active inflammatory response such as adult respiratory distress syndrome and in ischaemia-reperfusion injury. Intravenous lignocaine has also been shown to be effective in prevention of deep vein thrombosis after elective hip surgery⁷⁰. Lignocaine therefore appears to be effective in both attenuating inflammatory and hypercoagulable states (post-operative thrombosis) in the clinical setting^{70,71}. Unlike adenosine, lignocaine has not been implicated in the preconditioning of a cell, tissue or organ.

As lignocaine acts as a local anaesthetic by primarily blocking sodium fast channels, it will be appreciated that other sodium channel blockers could be used instead of or in combination with the local anaesthetic in the method and composition of the present invention. It will be appreciated that sodium channel blockers include compounds that substantially block sodium channels and also downregulate sodium channels. Examples of suitable sodium channel blockers include venoms such as tetrodotoxin, and the drugs primaquine, QX, HNS-32 (CAS Registry # 186086-10-2), NS-7, kappa-opioid receptor agonist U50 488, crobenetine, pilsicamide, phenyloin, tocamide, mexiletine, RS100642, riluzole, carbamazepine, flecamide, propafenone, amiodarone, sotalol, imipramine and moricizine, or any of derivatives thereof. Other suitable sodium channel blockers include: Vinpocetine (ethyl apovincaminate); and Beta-carboline derivative, nootropic beta-carboline (ambocarb, AMB).

Lidocaine in addition to being a local anaesthetic also has anti-inflammatory properties. Although the beneficial clinical effect of local anaesthetics and the regulation of the immune system remain poorly defined, studies have suggested several mechanisms of action including inhibition of the adhesion of granulocytes to the inflammatory sites, reduction of lysosomal activity, decreased production of superoxide and the suppression of metabolic activation and secretion of LTB4 and IL-1 from granulocytes. Lidocaine-related local anaesthetics have been shown to inhibit lymphocyte maturation and proliferation, inhibit the migration of macrophages into tissues, inhibit the expression of CD11b/CD18 by polymorphonuclear cells, inhibit the adhesion of leucocytes to injured venules and inhibit the LPS-stimulated secretion of LTB4 and IL-1 from peripheral blood mononuclear cells. Lidocaine's actions have also been linked to lidocaine-induced reduction in the release of substance P from nerve terminals.

Since polarisation of the membrane potential of tissue cell is one of the key factors involved in superior arrest, protection and preservation, we reasoned that adenosine and lidocaine may act synergistically to further produce enhanced inhibition of inflammation.

In another embodiment of the present invention there is provided a composition according to the present invention, further including an effective amount of an antioxidant.

Antioxidants are commonly enzymes or other organic substances that are capable of counteracting the damaging effects of oxidation in the tissue. The antioxidant component of the composition according to the present invention may be selected from one or more of the group consisting of: allopurinol, carnosine, histidine, Coenzyme Q 10, n-acetyl-cysteine, superoxide dismutase (SOD), glutathione reductase (GR), glutathione peroxidase (GP) modulators and regulators, catalase and the other metalloenzymes, NADPH and AND(P)H oxidase inhibitors, glutathione, U-74006F, vitamin E, Trolox (soluble form of vitamin E), other tocopherols (gamma and alpha, beta, delta), tocotrienols, ascorbic acid, Vitamin C, Beta-Carotene (plant form of vitamin A), selenium, Gamma Linoleic Acid (GLA), alpha-lipoic acid, uric acid (urate), curcumin, bilirubin, proanthocyanidins, epigallocatechin gallate, Lutein, lycopene, bioflavonoids, polyphenols, trolox(R), dimethylthiourea, tempol(R), carotenoids, coenzyme Q, melatonin, flavonoids, polyphenols, aminoindoles probucol and nitecapone, 21-aminosteroids or lazaroids, sulphydryl-containing compounds (thiazolidine, Ebselen, dithiolethiones), and N-acetylcysteine. Other antioxidants include the ACE inhibitors (captopril, enalapril, lisinopril) which are used for the treatment of arterial hypertension and cardiac failure on patients with myocardial infarction. ACE inhibitors exert their beneficial effects on the reoxygenated myocardium by scavenging reactive oxygen species. Other antioxidants that could also be used include beta-mercaptopropionylglycine, O-phenanthroline, dithiocarbamate, selegilize and desferrioxamine (Desferal), an iron chelator, has been used in experimental infarction models, where it exerted some level of antioxidant protection. Spin trapping agents such as 5′-5-dimethyl-1-pyrrolione-N-oxide (DMPO) and (a-4-pyridyl-1-oxide)-N-t-butylnitrone (POBN) also act as antioxidants. Other antioxidants include: nitrone radical scavenger alpha-phenyl-tert-N-butyl nitrone (PBN) and derivatives PBN (including disulphur derivatives); N-2-mercaptopropionyl glycine (MPG) a specific scavenger of the OH free radical; lipooxygenase inhibitor nordihydroguaretic acid (NDGA); Alpha Lipoic Acid; Chondroitin Sulfate; L-Cysteine; oxypurinol and Zinc.

Preferably, the antioxidant is allopurinol (1H-Pyrazolo[3,4-α]pyrimidine-4-ol). Allopurinol is a competitive inhibitor of the reactive oxygen species generating enzyme xanthine oxidase. Allopurinol's antioxidative properties may help preserve myocardial and endothelial functions by reducing oxidative stress, mitochondrial damage, apoptosis and cell death.

In addition, protease inhibitors attenuate the systemic inflammatory response in patients undergoing cardiac surgery with cardiopulmonary bypasss, and other patients where the inflammatory response has been heightened such as AIDS or in the treatment of chronic tendon injuries. Some broad spectrum protease inhibitors such as aprotinin also reduce blood loss and need for blood transfusions in surgical operations such as coronary bypass.

In another embodiment of the present invention there is provided a composition according to the present invention, further including an effective amount of a sodium hydrogen exchange inhibitor. The sodium hydrogen exchange inhibitor reduces sodium and calcium entering the cell.

The sodium hydrogen exchange inhibitor may be selected from one or more of the group consisting of amiloride, cariporide, eniporide, triamterene and EMD 84021, EMD 94309, EMD 96785 and HOE 642 and T-162559 (inhibitors of the isoform 1 of the Na⁺/H⁺ exchanger). Preferably, the sodium hydrogen exchange inhibitor is amiloride. Amiloride inhibits the sodium proton exchanger (Na⁺/H⁺ exchanger, also often abbreviated NHE-1) and reduces calcium entering the cell. During ischaemia excess cell protons (or hydrogen ions) are exchanged for sodium via the Na⁺/H⁺ exchanger.

Accordingly another aspect of the invention provides a method for preconditioning, arresting, protecting and/or reducing damage to a tissue during ischemia or reperfusion comprising delivery of an effective amount of:

a Na⁺/H⁺ exchange inhibitor; and

a local anaesthetic.

According to this aspect there is also provided a composition comprising:

a Na⁺/H⁺ exchange inhibitor; and

a local anaesthetic.

Preferably the Na⁺/H⁺ exchange inhibitor is Amiloride.

In yet another embodiment of the present invention there is provided a composition according to the present invention, further including an effective amount of:

a source of magnesium in an amount for increasing the amount of magnesium in a cell in the tissue; and

a source of calcium in an amount for increasing the amount of calcium in a cell in the tissue.

Elevated magnesium and low calcium has been associated with protection during ischaemia and reoxygenation of the organ. The action is believed due to decreased calcium loading.

Preferably the magnesium is present at a concentration of between 0.5 mM to 20 mM, more preferably about 2.5 mM. Preferably the calcium present is at a concentration of between 0.1 mM to 2.5 mM, more preferably about 0.3 mM.

In another aspect there is also provided a composition according to the invention further including an effective amount of elevated magnesium.

The composition according to the invention may also include an impermeant or a compound for minimizing or reducing the uptake of water by a cell in a tissue.

Compounds for minimizing or reducing the uptake of water by a cell in a tissue are typically impermeants or receptor antagonists or agonists.

A compound for minimizing or reducing the uptake of water by a cell in the tissue tends to control water shifts, ie, the shift of water between the extracellular and intracellular environments. Accordingly, these compounds are involved in the control or regulation of osmosis. One consequence is that a compound for minimizing or reducing the uptake of water by a cell in the tissue reduces cell swelling that is associated with Oedema, such as Oedema that can occur during ischemic injury.

An impermeant according to the present invention may be selected from one or more of the group consisting of: sucrose, pentastarch, hydroxyethyl starch, raffinose, mannitol, gluconate, lactobionate, and colloids. Colloids include albumin, hetastarch, polyethylene glycol (PEG), Dextran 40 and Dextran 60. Other compounds that could be selected for osmotic purposes include those from the major classes of osmolytes found in the animal kingdom including polyhydric alcohols (polyols) and sugars, other amino acids and amino-acid derivatives, and methylated ammonium and sulfonium compounds.

Cell swelling can also result from an inflammatory response which may be important during organ retrieval, preservation and surgical grafting. Substance P, an important pro-inflammatory neuropeptide is known to lead to cell oedema and therefore antagonists of substance P may reduce cell swelling. Indeed antagonists of substance P, (-specific neurokinin-1) receptor (NK-1) have been shown to reduce inflammatory liver damage, i.e., oedema formation, neutrophil infiltration, hepatocyte apoptosis, and necrosis. Two such NK-1 antagonists include CP-96,345 or [(2S,3S)-cis-2-(diphenylmethyl)-N-((2-methoxyphenyl)-methyl)-1-azabicyclo(2.2.2.)-octan-3-amine (CP-96,345)] and L-733,060 or [(2S,3S)3-([3,5-bis(trifluoromethyl)phenyl]methoxy)-2-phenylpiperidine]. R116301 or [(2R-trans)-4-[1-[3,5-bis(trifluoromethyl)benzoyl]-2-(phenylmethyl)-4-piperidinyl]-N-(2,6-dimethylphenyl)-1-acetamide (S)-Hydroxybutanedioate] is another specific, active neurokinin-1 (NK(1)) receptor antagonist with subnanomolar affinity for the human NK(1) receptor (K(i): 0.45 nM) and over 200-fold selectivity toward NK(2) and NK(3) receptors. Antagonists of neurokinin receptors 2 (NK-2) that may also reduce cell swelling include SR48968 and NK-3 include SR142801 and SB-222200. Blockade of mitochondrial permeability transition and reducing the membrane potential of the inner mitochondrial membrane potential using cyclosporin A has also been shown to decrease ischemia-induced cell swelling in isolated brain slices. In addition glutamate-receptor antagonists (AP5/CNQX) and reactive oxygen species scavengers (ascorbate, Trolox®, dimethylthiourea, Tempol®) also showed reduction of cell swelling. Thus, the compound for minimizing or reducing the uptake of water by a cell in a tissue can also be selected from any one of these compounds.

It will also be appreciated that the following energy substrates can also act as impermeants. Suitable energy substrate can be selected from one or more from the group consisting of: glucose and other sugars, pyruvate, lactate, glutamate, glutamine, aspartate, arginine, ectoine, taurine, N-acetyl-beta-lysine, alanine, proline and other amino acids and amino acid derivatives, trehalose, floridoside, glycerol and other polyhydric alcohols (polyols), sorbitol, myo-innositol, pinitol, insulin, alpha-keto glutarate, malate, succinate, triglycerides and derivatives, fatty acids and carnitine and derivatives.

Preferably the compound for minimizing or reducing the uptake of water by the cells in the tissue is sucrose. Sucrose reduces water shifts as an impermeant. Impermeant agents such as sucrose, lactobionate and raffinose are too large to enter the cells and hence remain in the extracellular spaces within the tissue and resulting osmotic forces prevent cell swelling that would otherwise damage the tissue, which would occur particularly during storage of the tissue.

Preferably, the concentration of the compound for minimizing or reducing the uptake of water by the cells in the tissue is between about 5 to 500 mM. Typically this is an effective amount for reducing the uptake of water by the cells in the tissue. More preferably, the concentration of the compound for reducing the uptake of water by the cells in the tissue is between about 20 and 100 mM. Even more preferably the concentration of the compound for reducing the uptake of water by the cells in the tissue is about 70 mM.

In another preferred embodiment of the present invention, there is provided a composition according to the present invention including an effective amount of:

a potassium channel opener and/or adenosine receptor agonist; and

a local anaesthetic,

and further including an effective amount of one or more components selected from:

diazoxide;

an opioid

an antioxidant;

an anti-adrenergic

a sodium hydrogen exchange inhibitor;

a calcium channel blocker

a source of magnesium; and

a source of calcium.

The term “tissue” is used herein in its broadest sense and refers to any part of the body exercising a specific function including organs and cells or parts thereof, for example, cell lines or organelle preparations. Other examples include conduit vessels such as arteries or veins or circulatory organs such as the heart, respiratory organs such as the lungs, urinary organs such as the kidneys or bladder, digestive organs such as the stomach, liver, pancreas or spleen, reproductive organs such as the scrotum, testis, ovaries or uterus, neurological organs such as the brain, germ cells such as spermatozoa or ovum and somatic cells such as skin cells, heart cells (ie, myocytes), nerve cells, brain cells or kidney cells.

It will be understood that the term “comprises” or its grammatical variants as used in this specification and claims is equivalent to the term “includes” and is not to be taken as excluding the presence of other elements or features.

The composition of the present invention is particularly useful in preconditioning, arresting, protecting and/or preserving the heart during open-heart surgery including heart transplants. Other applications include reducing heart damage before, during or following cardiovascular intervention which may include a heart attack, “beating heart” surgery, angioplasty or angiography. For example, the composition could be administered to subjects who have suffered or are developing a heart attack and used at the time of administration of blood clot-busting drugs such as streptokinase. As the clot is dissolved, the presence of the composition may protect the heart from further injury such as reperfusion injury. The composition may be particularly effective as a cardioprotectant in those portions of the heart that have been starved of normal flow, nutrients and/or oxygen for different periods of time. For example, the composition may be used to treat heart ischaemia which could be pre-existing or induced by cardiovascular intervention.

In a preferred embodiment the composition according to the present invention is a cardioplegic and/or cardioprotectant composition.

According to another aspect of the present invention there is provided use of the composition according to the present invention in the manufacture of a medicament for preconditioning, arresting, protecting and/or preserving an organ.

In a preferred embodiment of this aspect of the present invention it is preferred to aerate the composition with a source of oxygen before and/or during use. The source of oxygen may be an oxygen gas mixture where oxygen is the predominant component. The oxygen may be mixed with, for example CO₂. Preferably, the oxygen gas mixture is 95% O₂ and 5% CO₂.

It is considered that the oxygenation with the oxygen gas mixture maintains mitochondrial oxidation and this helps preserve the myocyte and endothelium of the tissue.

In another aspect of the present invention there is provided a method for preconditioning, arresting, protecting and/or preserving a tissue including:

providing in a suitable container a composition according to the invention and a source of oxygen;

aerating the composition with the oxygen; and

placing the tissue in contact with the composition under conditions sufficient to precondition arrest, protect and/or preserve thereof.

In another embodiment of the present invention there is provided use of a composition according to the invention for preconditioning, arresting, protecting and/or preserving a tissue, wherein the composition is aerated with the oxygen and contacts the organ.

Preferably the oxygen source is an oxygen gas mixture. Preferably oxygen is the predominant component. The oxygen may be mixed with, for example CO₂. More preferably, the oxygen gas mixture is 95% O₂ and 5% CO₂.

Preferably the composition is aerated before and/or during contact with the tissue.

Preferably the composition according to this aspect of the invention is in liquid form. Liquid preparations of the composition may take the form of, for example, solutions, syrups, or suspensions, or may be presented as a dry product for constitution with water or other suitable vehicle. Such liquid preparations may be prepared by conventional means with pharmaceutically acceptable additives such as suspending agents, emulsifying agents, non-aqueous vehicles, preservatives and energy sources.

While the present invention is particularly advantageous in preconditioning, arresting, protecting and/or preserving an organ while intact in the body of a subject, for example in the treatment of the heart in circumstances of myocardial infarction or heart attack, it will also be appreciated that the present invention may also be used to arrest, protect and/or preserve isolated organs.

The subject from which the tissue is to be preconditioned, arrested, protected and/or preserved may be a human or an animal such as a livestock animal (eg, sheep, cow or horse), laboratory test animal (eg, mouse, rabbit or guinea pig) or a companion animal (eg, dog or cat), particularly an animal of economic importance.

The method of the present invention involves contacting a tissue with the composition, for a time and under conditions sufficient for the tissue to be preconditioned, arrested, protected and/or preserved.

The composition may be infused or administered as a bolus intravenous, intracoronary or any other suitable delivery route as pre-treatment for protection during a cardiac intervention such as open heart surgery (on-pump and off-pump), angioplasty (balloon and with stents or other vessel devices) and as with clot-busters (ant-clotting drug or agents).

The composition can also be infused or administered as a bolus intravenous, intracoronary or any other suitable delivery route for protection during cardiac intervention such as open heart surgery (on-pump and off-pump), angioplasty (balloon and with stents or other vessel devices) and as with clot-busters to protect and preserve the cells from injury.

The composition may also be infused or administered as a bolus intravenous, intracoronary or any other suitable delivery route for protection following a cardiac intervention such as open heart surgery (on-pump and off-pump), angioplasty (balloon and with stents or other vessel devices) and as with clot-busters to protect and preserve the cells from injury.

Accordingly, the tissue may be contacted by delivering the composition according to the invention intravenously to the tissue. This involves using the blood as a vehicle for delivery to the tissue. In particular, the composition according to the invention may be used for blood cardioplegia.

Alternatively, the composition may be delivered directly to the tissue for affecting the viability of the tissue. In particular, the composition according to the invention may be used for crystalloid cardioplegia.

The composition according to the invention may be delivered according to one of or a combination of the following delivery protocols: intermittent, continuous and bolus.

The dose and time intervals for each delivery protocol may be designed accordingly. For example, a composition according to the invention may be delivered as a bolus to the tissue to initially arrest the tissue. A further composition according to the invention may then be administered continuously to maintain the tissue in an arrested state. Yet a further composition according to the invention may be administered continuously to reperfuse the tissue or recover normal function.

Accordingly, in another aspect of the invention, there is provided a composition for arresting, protecting and preserving a tissue upon administration of a bolus or single dose of the composition, the composition including a primary potassium channel opener or agonist and/or adenosine receptor agonist and a local anaesthetic. The invention also provides a method for arresting and protecting an tissue comprising administering as a single dose an effective amount of that composition. A bolus or single dose administration may also be referred to as a “one-shot” administration.

In another aspect of the invention, there is provided a composition for arresting, protecting and preserving a tissue by intermittent administration of the composition, the composition including an effective amount of a primary potassium channel opener or agonist and/or adenosine receptor agonist and a local anaesthetic. A suitable administration schedule is a 2 minute induction dose every 20 minutes throughout the arrest period. The actual time periods can be adjusted based on observations by one skilled in the art administering the composition, and the animal/human model selected. The invention also provides a method for intermittently administering a composition for arresting, protecting and preserving a tissue.

The composition can of course also be used in continuous infusion with both normal and injured tissues or organs, such as heart tissue. Continuous infusion also includes static storage of the tissue, whereby the tissue is stored in a composition according to the invention, for example the tissue may be placed in a suitable container and immersed in a solution according to the invention for transporting donor tissues from a donor to recipient.

The dose and time intervals for each delivery protocol may be designed accordingly. For example, a composition according to the invention may be delivered as a one-shot to the tissue to initially arrest of the tissue. A further composition according to the invention may then be administered continuously to maintain the tissue in an arrested state. Yet a further composition according to the invention may be administered continuously to reperfuse the tissue or recover normal function.

As mentioned previously, the composition according to the invention may be used at a temperature range selected from one of the following: from about 0° C. to about 5° C., from about 5° C. to about 20° C., from about 20° C. to about 32° C. and from about 32° C. to about 38° C. It is understood that “profound hypothermia” is used to describe a tissue at a temperature from about 0° C. to about 5° C. “Moderate hypothermia” is used to describe a tissue at a temperature from about 5° C. to about 20° C. “Mild hypothermia” is used to describe a tissue at a temperature from about 20° C. to about 32° C. “Normothermia” is used to describe a tissue at a temperature from about 32° C. to about 38° C.

The composition according to the present invention is highly beneficial at about 10° C. but can also arrest preserve and protect over a wider temperature range up to about 37° C. In contrast, the majority of present day arrest and preservation solutions operate more effectively at lower temperatures the longer arrest times using St Thomas No. 2 solution may only be achieved when the temperature is lowered, for example, to a maximum of 4° C. Moreover, the composition according to the invention may be used at a temperature range selected from the following: 0° C. to 5° C., 5° C. to 20° C., 20° C. to 32° C. and 32° C. to 38° C.

In another aspect of the invention, there is provided a method of reducing heart tissue damage during a heart attack, cardioplegia or event likely to be ischaemic for a particular tissue or tissues by delivering a composition to the tissue, the composition according to the composition of the invention together with a suitable carrier or excipient, such as for example physiological saline or blood. In particular, there is provided a method of blood cardioplegia and crystalloid cardioplegia.

In another aspect of the invention, there is provided a method of protecting heart tissue from reperfusion injury, including inflammatory and blood clotting and coagulation effects often experienced during reperfusion following an ischaemic event, such as in the post-operative period or longer-term recovery. The method comprises administering a solution comprising the composition according to the present invention.

The invention also provides a method for reducing infarction size and/or reducing inflammation and blood coagulation responses in heart tissue during ischaemia and/or reperfusion comprising administration of the same solution.

While it is possible for each component of the composition to contact the tissue alone, it is preferable that the components of the composition be provided together with one or more pharmaceutically acceptable carriers, diluents, adjuvants and/or excipients. Each carrier, diluent, adjuvant and/or excipient must pharmaceutically acceptable such that they are compatible with the components of the composition and not harmful to the subject. Preferably, the composition is prepared with liquid carriers, diluents, adjuvants and/or excipients.

The composition according to the invention may be suitable for topical administration to the tissue. Such preparation may be prepared by conventional means in the form of a cream, ointment, jelly, solution or suspension.

The composition may also be formulated as depot preparations. Such long acting formulations may be administered by implantation (eg, subcutaneously or intramuscularly) or by intramuscular injection. Thus, for example, the composition according to the invention may be formulated with suitable polymeric or hydrophobic materials (eg, as an emulsion in an acceptable oil or ion exchange resins, or as sparingly soluble derivatives, for example, as a sparingly soluble salt.

Accordingly, this aspect of the invention also provides a method for preconditioning, arresting, protecting and/or preserving an organ, which includes providing the composition together with a pharmaceutically acceptable carrier, diluent, adjuvant and/or excipient.

A preferred pharmaceutically acceptable carrier is a buffer having a pH of about 6 to about 9, preferably about 7, more preferably about 7.4 and/or low concentrations of potassium, for example, up to about 10 mM, more preferably about 2 to about 8 mM, most preferably about 4 to about 6 mM. Suitable buffers include Krebs-Henseleit which generally contains 10 mM glucose, 117 mM NaCl, 5.9 mM KCl, 25 mM NaHCO₃, 1.2 mM NaH₂PO₄, 1.12 mMCaCl₂ (free Ca²⁺=1.07 mM) and 0.512 mM MgCl₂ (free Mg²⁺=0.5 mM), St. Thomas No. 2 solution, Tyrodes solution which generally contains 10 mM glucose, 126 mM NaCl, 5.4 mM KCl, 1 mM CaCl₂, 1 mM MgCl₂, 0.33 mM NaH₂PO₄ and 10 mM HEPES (N-[2-hydroxyethyl]piperazine-N′-[2-ethane sulphonic acid], Fremes solution, Hartmanns solution which generally contains 129 NaCl, 5 mM KCl, 2 mM CaCl₂ and 29 mM lactate and Ringers-Lactate. Other naturally occurring buffering compounds that exist in muscle that could be also used in a suitable ionic environment are carnosine, histidine, anserine, ophidine and balenene, or their derivatives. Very recent studies have suggested that the processes of inflammation and thrombosis are linked through common mechanisms. Therefore, it is believed that understanding of the processes of inflammation will help with better management of thrombotic disorders including the treatment of acute and chronic ischaemic syndromes. In the clinical and surgical settings, a rapid response and early intervention to an organ or tissue damaged from ischemia, can involve both anti-inflammatory and anti-cloffing therapies. In addition to protease inhibitors which have been shown to attenuate the inflammatory response, further anti-inflammatory therapies have included the administration of aspirin, normal heparin, low-molecular-weight heparin (LMWH), non-steroidal anti-inflammatory agents, anti-platelet drugs and glycoprotein (GP) IIb/IIIa receptor inhibitors, statins, angiotensin converting enzyme (ACE) inhibitor and angiotensin blockers. Examples of protease inhibitors are indinavir, nelfinavir, ritonavir, lopinavir, amprenavir or the broad-spectrum protease inhibitor aprotinin, a low-molecular-weight heparin (LMWH) is enoxaparin, non-steroidal anti-inflammatory agent are indomethacin, ibuprofen, rofecoxib, naproxen or fluoxetine, an anti-platelet drug is Clopidogrel, a glycoprotein (GP) IIb/IIIa receptor inhibitor is abciximab, a statin is pravastatin, an angiotensin converting enzyme (ACE) inhibitor is captopril and an angiotensin blocker is valsartin. Another example of an anti-platelet drug is Aspirin.

In another embodiment of the present invention there is provided use of a composition for preconditioning, arresting, protecting and/or preserving an organ including an effective amount of:

a potassium channel opener and/or adenosine receptor agonist and;

a local anaesthetic;

provided in a suitable container together with a source of oxygen;

wherein the composition is aerated with the oxygen and contacts the organ.

In another preferred embodiment of the present invention there is also provided a reperfusion solution which is administered after arrest particularly long-term arrest, protection and preservation, together with the solution according to the invention.

Preferably, the reperfusion solutions comprises Krebs Henseleit buffer.

Preferably, the reperfusion solution is provided at 37° C.

The composition according to the invention may also include an energy substrate. The energy substrate helps with recovering metabolism. The energy substrate can be selected from one or more components selected from the group consisting of: glucose and other sugars, pyruvate, lactate, glutamate, glutamine, aspartate, arginine, ectoine, taurine, N-acetyl-beta-lysine, alanine, proline and other amino acids and amino acid derivatives, trehalose, floridoside, glycerol and other polyhydric alcohols (polyols), sorbitol, myo-innosotil, pinitol, insulin, alpha-keto glutarate, malate, succinate, trigylcerides and derivatives, fatty acids and carnitine and derivatives.

Throughout this specification, unless stated otherwise, where a document, act or item of knowledge is referred to or discussed, this reference or discussion is not an admission that the document, act or item of knowledge, or any combination thereof, at the priority date, was part of the common general knowledge.

### DESCRIPTION OF FIGURES

The invention will now be described with reference to the following examples. These examples are not to be construed as limiting in any way. In this description, there are the following figures.

FIG. 1. illustrates diagrammatically the experimental design for combinatorial therapy of Adenosine and Lignocaine after regional ischemia at varying concentrations.

FIG. 2. is a graph comparing arrhythmic deaths from ventricular fibrillation during LCA occlusion comparing varying compositions according to the invention.

FIG. 3. are graphs comparing episodes (A) and duration (B) of ventricular tachycardia (VT) and ventricular fibrillation (VF) and VT+VF during ischaemia for surviving rats in all treatment groups. These values represent overall sum of episodes and durations (sec) that occurred throughout the 30 min ischaemic period. The percentage of animals that experienced either VT or VF per group are shown. Surviving rats: saline-control, n=5, AL solution, n=7; Ado-only, n=4; Lido-only, n=6. *P<0.05 vs. control, †P<0.05 vs AL-I group.

FIG. 4. are graphs comparing effects of AL mixture (“Al-soln”), adenosine alone (“Ado-only”) or lidocaine alone (“Lido-only”) treatments on left ventricle necrosis and infarct size. Areas at risk (AAR/LV) were not significantly different between groups (A). Areas of necrosis in the left ventricle (AN/LV) were significantly smaller with AL mixture treatment (B). Infarct sizes (AN/AAR) in groups receiving AL solution treatment were significantly smaller compared with all other treatment groups (C). Only data from Surviving rats are shown: saline-control, n=5; AL soln, n=7; Ado-only, n=4; Lido-only, n=6. *P<0.05 vs. control, ÝP<0.05 vs AL-soln group. The black filled-in squares represent the mean±SEM for each of the groups whereas the open symbols represent the values for each animal in that group.

FIG. 5. are graphs comparing Hemodynamic changes for all surviving animals during the course of the experiment described in Example 1. Measurements were recorded throughout pretreatment/preocclusion, ischaemia and reperfusion. Shown above are in order of appearance: equilibration, following 5 min pretreatment, 10, 20 and 30 min ischaemia and every 20 min through out reperfusions. All groups received treatment through 30 min ischaemia. (A) Heart rate (HR); (B) Mean arterial pressure (MAP); (C) Rate-pressure product (RPP). Large symbols represent means±SE for each group. Surviving rats: saline-control, n=5; AL solution, n=7; Ado-only, n=4; Lido-only, n=6.

FIG. 6. are Scatterplots of the relationship of MAP (A) and RPP (B) and infarct size following pretreatment prior to ischaemia as described in Example 1. Negative values connote the decline in the measured points. Following pretreatment, a correlation was found between infarct size and all hemodynamic variables in the Ado-only treatment group.

FIG. 7. show graphs comparing the episodes (A) and duration (B) of ventricular tachycardia (VT) and ventricular fibrillation (VF) and VT+VF during ischaemia for surviving rats of second study as described in Example 1. These values represent overall sum of episodes and durations (sec) that occurred throughout the 30 min ischaemic period. Surviving rats: saline-control, n=5; AL solution, n=7; Lido, Ado-SEQ, n=5; AL SEQ, n=6, AL-Pre-I-Rep, n=6. *P<0.05 vs. control, †P<0.05 vs AL solution group.

FIG. 8. shows graphs comparing the affects of AL solution and sequential administration of adenosine and lignocaine during ischaemia and/or reperfusion on infarct size as described in Example 1. (A) Areas at risk (AAR) were not significantly different between groups. (B) Areas of necrosis (AN/LV) in the left ventricle were reduced with AL solution treatment in comparison all groups tested (C). Infarct sizes (AN/AAR) in groups receiving AL treatment were significantly smaller compared with all other groups. Surviving rats: saline-control, n=5; AL solution, n=7; Lido, Ado-SEQ, n=5; AL SEQ, n=6, AL-Pre-I-Rep, n=6. *P<0.05 vs. Control; †P≦0.05 vs. AL solution.

FIG. 9. is a graph comparing percentage deaths from ventricular fibrillation during ischemia from different compositions according to the invention as described in Example 2. The actual percentage of animals that died per group is shown above bars. The total number of rats in each group are as follows: Saline-control, n=12; IPC, n=6; AL soin, n=7; A1 agonist (CCPA, 5 μg/kg) plus lido; n=6 A1 agonist only (CCPA, 5 μg/kg), n=7.

FIG. 10. is a graph comparing episodes and duration of ventricular tachycardia (VT) and ventricular fibrillation (VF) and VT+VF during ischemia for surviving rats in all treatment groups as described in Example 2. These values represent the overall sum of episodes and durations (sec) that occurred throughout the 30 min ischaemic period. Surviving rats: saline-control, n=5; IPC, n=5, AL soln, n=7, A1 agonist (CCPA, 5 μg/kg) plus lido, n=6 A1 agonist only (CCPA, 5 μg/kg), n=5. *P<0.05 vs. control; †P<0.05 vs. IPC.

FIG. 11. show graphs comparing the effects of solutions according to the invention AL soln, A1 agonist (CCPA) plus lidocaine, and A1 agonist (CCPA) only to “IPC” on left ventricle necrosis and infarct size as described in Example 2. Areas at risk (MR/LV) were not significantly different between groups (A). Areas of necrosis in the left ventricle (AN/LV) were significantly smaller with AL mixture treatment (A). Infarct sizes (AN/AAR) in groups receiving AL solution treatment were significantly smaller compared with all other treatment groups (B). Surviving rats: Saline-control, n=5; IPC, n=5; AL soln, n=7; A1 agonist (CCPA, 5 μg/kg) plus lido, n=6; A1 agonist only (CCPA, 5 μg/kg), n=5. *P<0.05 vs. control.

FIG. 12. illustrates a possible scheme of adenosine and lidocaine's possible mutiple signaling mechanisms involved in early (classic) preconditioning of the in situ rat myocardium and coronary microvascular. Co-administering adenosine (or adenosine agonists) plus lidocaine target electrophysiological (nodal, intercalated discs, myocyte), mechanical and metabolic sites which lead to substantial protection against mortality, life-threatening arrhythmias and tissue necrosis. Delayed protection is due in part to improved atrial and ventricular matching of electrical conduction and pump performance. Targeting adenosine receptors and voltage sensitive Na⁺ fast channels may offer a new therapeutic window to delay myocardial damage during ischemia-reperfusion. Abbreviations used: AP, action potential; AV, atrioventricular; Gi/o, inhibitory membrane bound G protein which couples adenosine receptors to intracellular signaling pathways; PKC, protein kinase C; IK_{Ach/Ado}, inwardly rectifying K⁺-channel current which in supraventricular tissue (e.g., AV nodal myocytes) is directly linked to activation by adenosine/A1 activation (cAMP independent)—hyperkalemia potentiate slowing AV nodal conduction⁴⁰; SR, sarcoplasmic reticulum; ROS, reactive oxygen species which in small amounts may serve as signal transduction messengers; cyclic AMP, cyclic adenosine monophosphate; AL, adenosine and lidocaine.

FIG. 13. contains Table 2, being the results of functional parameters of isolated working rat hearts during pre-arrest and reperfusion following 30 minute arrest with a composition according to the invention including Adenosine (200 nM), Lidocaine (500 uM) and Esmolol (100 uM). (in 10 mM glucose containing Krebs Henseleit, pH 7.55 delivered intermittently at 37° C.).

FIG. 14. contains Table 3, being the results of functional parameters of isolated working rat hearts during pre-arrest and reperfusion following 30 minute arrest with a composition according to the invention including Adenosine (200 mM), Liguocaine (500 uM) and Esmolol (10 uM). (in 10 mM glucose containing Krebs Henseleit, pH 7.60 delivered intermittently at 37° C.)

FIG. 15. contains Table 4, being the results of functional parameters of isolated working rat hearts during pre-arrest and reperfusion following 30 minute arrest with a composition according to the invention including adenosine (20 mM), lidocaine (500 uM) and esmolol (100 uM) (in 10 mM glucose containing Krebs Henseleit, pH 7.51 delivered intermittently at 37° C.).

FIG. 16. contains Table 5, being the results of parameters of isolated working rat hearts during pre-arrest and reperfusion following 30 minute arrest with a composition according to the invention including nifedipine (0.44 uM) and lidocaine (500 uM).

FIG. 17. contains Table 6, being the results of functional parameters of isolated working rat hearts during pre-arrest and reperfusion following 30 minute arrest with a composition according to the invention including nifedipine (2 uM), adenosine (200 uM) and lidocaine (500 uM).

FIG. 18. contains Table 7, being the results of functional parameters of isolated working rat hearts during pre-arrest and reperfusion following 30 minute arrest with a composition according to the invention containg DPDPE (1 uM) and lidocaine (500 uM). (in 10 mM glucose containing Krebs Henseleit, pH 7.55 delivered intermittently at 37° C.). Note: % return refers to % of pre-arrest values.

FIG. 19. contains Table 8, being the results of functional parameters of isolated working rat hearts during pre-arrest and reperfusion following 30 minute arrest with a composition according to the invention containing DPDPE (1 uM), adenosine (200 uM) and 500 uM lidocaine. (in 10 mM glucose containing Krebs Henseleit, pH 7.55 delivered intermittently at 37° C.). Note: % return refers to % of pre-arrest values.

FIG. 20. illustrates the effect of A, L and AL on In Vitro Superoxide anion generation by activated neutrophils as described in Example 6.

FIG. 21. contains Table 9, being the results of functional parameters of “one shot” normothermic arrest in the isolated healthy working heart using a composition according to the invention containing Adenosine (200 uM) and Lidocaine (500 uM).

FIG. 22. contains Table 10, being the results of functional parameters of “one Shot” normothermic arrest in healthy working rat heart using a composition containing Adenosine (200 uM), Lidocaine (500 uM) and Nifedipine (52 uM).

FIG. 23. contains Table 11, being the results of functional parameters of continuous normothermic arrest in healthy working rat heart using a composition according to the invention containing Adenosine (200 uM) and Lidocaine (500 uM).

FIG. 24. contains Table 12 being The results of functional parameters of intermittent normothermic arrest in healthy working rat heart using a composition according to the invention containing Adenosine (200 uM) and Lidocaine (500 uM).

FIG. 25. contains Table 13, being the results of functional parameters of isolated working rat hearts during pre-arrest and reperfusion following 30 minute arrest with AL cardioplegia solution according to the invention containing high Magnesium (16 mM), high Chloride (158 mM) and normal Sodium (143 mM).

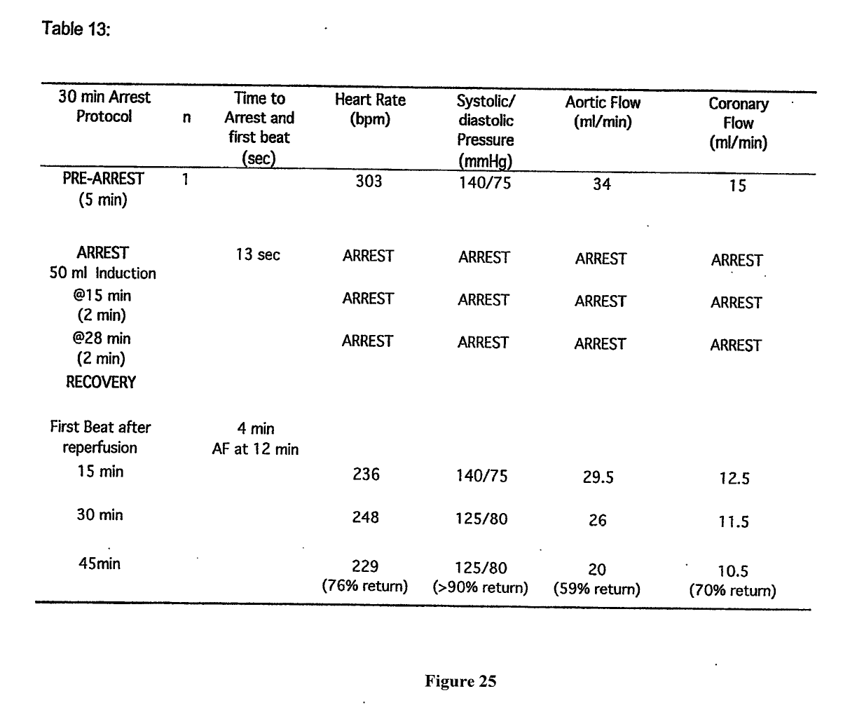

FIG. 26. contains Table 14, being the results of functional parameters of isolated working rat hearts during pre-arrest and reperfusion following 30 minute arrest with AL cardioplegia solution according to the invention containing high Magnesium (16 mM), normal Chloride (124.5 mM) and low Sodium (111 mM).

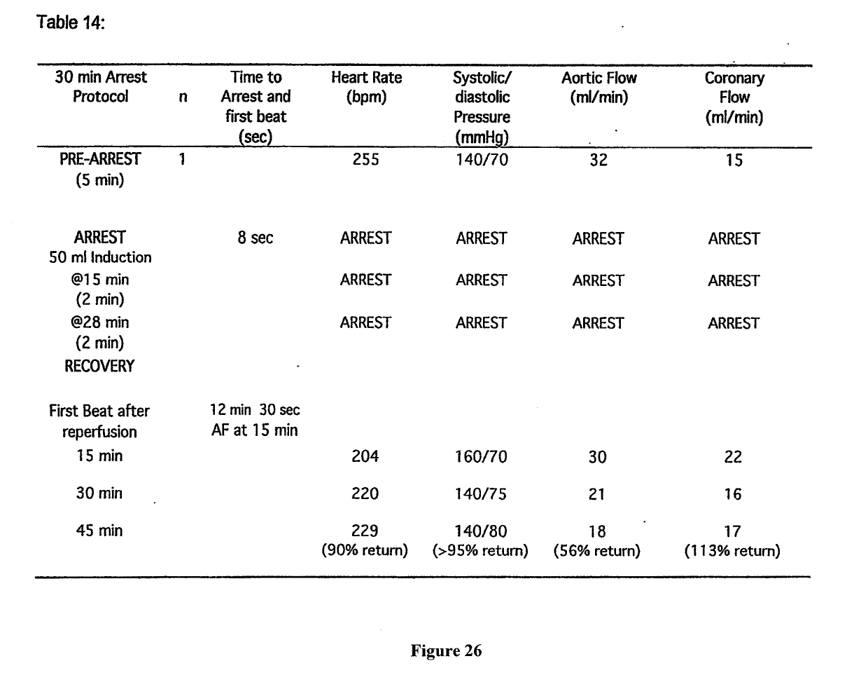

FIG. 27 is a graph comparing a composition according to the invention (“AL”) with a prior art composition (“St Thomas” or “ST”) by measuring Cardiac Output (as a percentage of pre-arrest output) at various times after arrest of injured rat hearts.

FIG. 28 is the same as FIG. 27 but measuring Aortic Flow recovery.

FIG. 29 is the same as FIG. 27 but measuring Coronary Flow.

FIG. 30 is the same as FIG. 27 but measuring Systolic Pressure.

FIG. 31 shows data for (a) Coronary Vascular Resistance (CVR) and (b) O₂ consumption measured during cardioplegia delivery at different times during 2 and 4 hr arrest of a healthy heart.

FIG. 32 is a representative profile of a heart's surface temperature during arrests.

FIG. 33 contains Table 15, being the estimates of the membrane potential in the isolated rat heart before and during arrest by adenosine and lidocaine cardioplegia, hyperkalemic St. Thomas Hospital solution No. 2 or 16 M KCl at 37° C.

FIG. 34 contains Table 16, being the results of functional recovery following 2 hrs arrest with the 2 hr arrest data reflected in FIG. 31.

FIG. 35 contains Table 17, being the results of functional recovery following 4 hrs arrest with the 4 hr arrest data reflected in FIG. 31.

### EXAMPLES

### Example 1

### Combinational Therapy of Adenosine and Lignocaine after Regional Ischemia (at Varying Concentrations)

Animals and Reagents: Male Sprague Dawley rats (330-400 g) from the James Cook University Breeding Colony were fed ad libitum and housed in a 12-hour light/dark cycle. On the day of the experiment rats were anaesthetised with an intraperitoneal injection of Nembutal (Sodium Pentabarbitone; 60 mg/kg) and the anaesthetic was administered as required throughout the protocol. Animals were treated in accordance with the James Cook University Guidelines for use of ‘Animals for Experimental Purposes’ (Ethics approval number A557). Adenosine (A9251 >99% purity), copper II pthalocyanine-tetrasulfonic acid tetrasodium salt (blue dye), and triphenyltetrazolium chloride (TTC) and all chemicals were obtained from Sigma Aldrich (Castle Hill, NSW). Lidocaine hydrochloride was purchased as a 2% solution (ilium) from the local Pharmaceutical Supplies (Lyppard, Queensland).

Surgical Protocol: Anesthetized animals were positioned in a specially designed plexiglass cradle. A tracheotomy was performed and the animals were artificially ventilated at 75-80 strokes per min on humidified room air using a Harvard Small Animal Ventilator (Harvard Apparatus, Mass., USA). Blood pO₂, pCO₂ and pH were maintained in the normal physiological range and measured on a Ciba-Corning 865 blood gas analyser. Body temperature was maintained at 37° C. using a homeothermic blanket control unit (Harvard Apparatus, Mass., USA). The left or right femoral vein was cannulated using PE-50 tubing for drug infusions while the left femoral artery was cannulated for blood collection and to monitor blood pressure (UFI 1050 BP) using a MacLab.

A left thoracotomy was performed through the 4^{th} and 5^{th} intercostals space. The pericardium was opened and the heart gently exteriorized. A 6-0 suture was threaded under the left coronary artery (LCA) located between the base of the pulmonary artery and left atrium. The LCA ties were attached to a custom designed snare occluder fastened to the cradle via a 20-inch teflon tube attached to a detachable 10 g weight. By adding or removing the weight, a constant ligation pressure could be applied and easily released. Leads were implanted subcutaneously in a lead II electrocardiogram (ECG) configuration. Rats were stabilised for 15-20 minutes prior to occlusion. Any animal that produced dysrhythmias or a sustained fall in mean arterial blood pressure below 80 mmHg was discarded from the study. Ischaemia was confirmed by regional cyanosis downstream of the occlusion and reperfusion was confirmed by lack of cyanosis in that region.

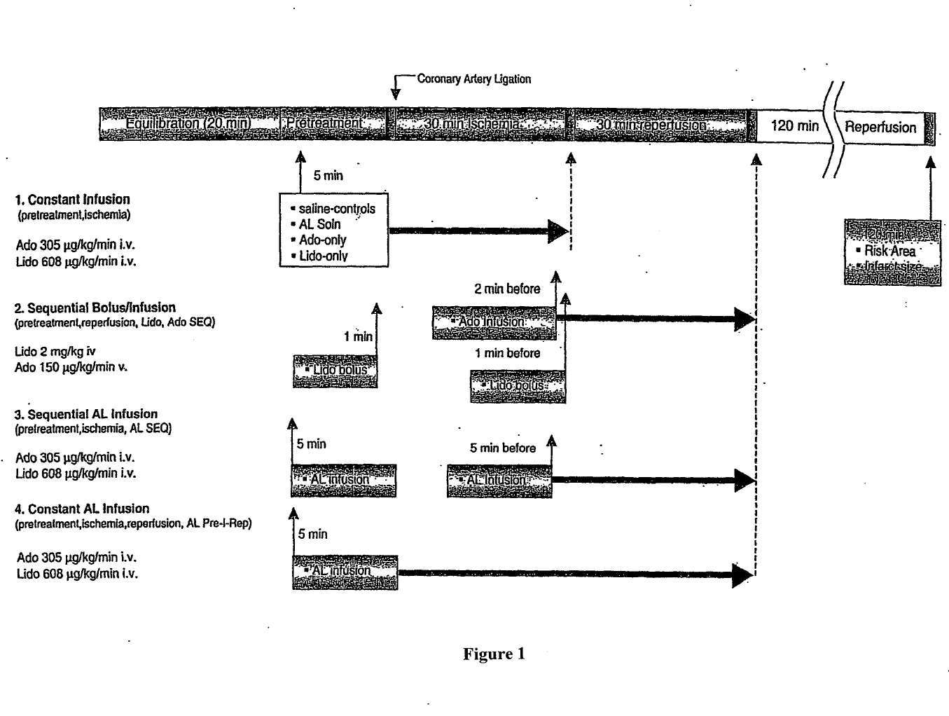

Experimental Design: The protocols are summarised below and in FIG. 1. The experimental work was conducted over a period of approximately three hours. This comprised 5 minutes “pre-treatment”, immediately following the 20 minute equilibration period referred to above. At the end of the pre-treatment period, the left coronary artery was ligated. This was maintained for 30 minutes to cause ischaemic conditions, followed by 30 minutes of reperfusion and another 2 hours of reperfusion, after which the risk area and infarct size measurements were taken. In Study I, the timing of administration of the compositions was a key parameter being tested. As shown in FIG. 1, and stated in the experimental methods, constant infusion was carried out during the 5 minutes of pre-treatment and the 30 minutes of ischaemia. This was the protocol used for infusion of saline controls, AL solution, Ado only (305 micrograms/kg/min iv) and Lido only (608 micrograms/kg/min iv). The other three administration protocols were as follows. “Sequential bolus/infusion” (“Lido, Ado SCQ”) involved a bolus dose of Lido at the end of the pre-treatment period (2 mg/kg iv) followed by another similar bolus at the end of the 30 minute ischaemic period. One minute before this second bolus, Ado infusion was commenced continuously through to the end of the 30 minute reperfusion period at 150 microgram/kg/min iv. “Sequential AL Infusion” (or “AL SEQ”) involved two infusions of AL solution, the first being for a 5 minute pre-treatment period, and the second being for about 35 minutes comprising the last 5 minutes of the 30 minutes of ischaemia and the 30 minutes of reperfusion (again using the 305 Ado and 608 Lido microgram/kg/min iv doses). Finally, “Constant AL Infusion” (or “AL Pre-I-Rep”) involved continuous AL infusion for a period of about 65 minutes from the beginning of the pre-treatment period to the end of the 30 minute reperfusion period at the same doses (Ado 305 and Lido 608 microgram/kg/min iv).

Study I: The adenosine and lidocaine solution (AL solution) contained 6.3 mg/ml adenosine (Ado) and 12.6 mg/ml lidocaine (Lido) and was prepared on the day of the experiment in physiological saline (0.9%). Drugs were infused intravenously at 1 ml/hr (210 infusion pump, Stoelting, Ill.), which convert to mass specific dosages of 305 μg/kg/ml/min and 608 μg/kg/ml/min for Ado and Lido respectively. In the first study, 36 animals were randomly assigned into 4 treatment groups: (1) Saline-controls (0.9% saline) (n=12); (2) AL solution (n=8), (3) Ado-only (305 μg/kg/ml/min, n=8); or (4) Lido-only (608 μg/kg/ml/min, n=8). All rats received continuous infusion for 5 minutes prior to and throughout 30 minutes of regional ischaemia. The treatment was ceased when the coronary ligature was released at the onset of reperfusion after 30 min ischaemia and animals reperfused for 120 minutes for infarct sizing.

Study II: Rats (n=19) were randomly assigned to one of three different treatment regimes: (1) Lido, Ado SEQ: a rapid bolus of lidocaine (2 mg/kg i.v.) given 1 min before LCA ligation and another bolus at 1 min before reperfusion. In addition, adenosine (150 μg/kg/ml/min) was infused 2 min before reperfusion and continued throughout 30 min of reperfusion (n=7). (2) AL SEQ: AL given at two separate times, 5 min before but not throughout ischaemia then 5 min before reperfusion and throughout 30 min reperfusion (n=6); (3) AL Pre-1-Rep: AL 5 min before and throughout ischaemia and 30 min reperfusion (n=6). These groups were compared to saline-controls and the AL solution group from Study I.

The primary end-points used to assess the cardioprotective effects of AL solution were infarct size, episodes and duration of ventricular arrhythmias and death. High mortality in the control group was observed in these pilot studies as is common in the rat model of acute myocardial ischaemia⁸⁴. On the basis of the binomial distribution for episode of ventricular fibrillation cited in the Lambeth Conventions, the study protocol required at least 4 animals in each group to survive for sufficient statistical power to test the primary end-points⁸⁵. The secondary end-points included heart rate, mean arterial pressure (systolic pressure−diastolic pressure/3+diastolic pressure) and rate pressure product (heart rate×systolic pressure).

Analysis of the ischaemic area at risk and infarct size: After 120 minutes reperfusion, the coronary artery was reoccluded and the heart excised. Blue dye (Copper (II) Pthalocyanine-tetrasulfonic acid Tetrasodium salt, 3 ml) was flushed retrograde through the aorta at a flow rate of approximately 18 ml/min and allowed to circulate through the coronary vasculature to delineate the ischaemic risk zone. The heart was sliced transversely into 6 or 7 slices of uniform thickness (2 mm) using a custom-made, equal spaced, multi-scalpel blade slicer. The slices were weighed and digitally photographed. Area measurements were made using the Image J (NIH) image analysis program. The area left unstained by the blue dye was defined as the left ventricular ‘area-at-risk’ (AAR/LV) while the blue-stained region was the perfused area not at risk of suffering ischaemic damage. The slices were then incubated in a 1% solution of triphenyltetrazolium chloride (TTC) at 37° C. for 15 min⁸⁶, immersed in formalin and photographed again. The area of necrosis in the left ventricle (AN/LV) was the region of the slice unstained by TTC (white) while the non-infarcted region was the area of the slice stained by TTC (brick red). Infarct size of the left ventricle was defined as the ratio of the area of necrosis (AN) to the area at risk (AN/AAR) and expressed as a percentage.

Arrhythmia Analysis: Arrhythmias were analysed separately during 30 min ischaemia and the first 30 min of reperfusion. Using the lead II ECG tracing, the episodes and duration of episodes of ventricular tachycardia (VT) and ventricular fibrillation (VF) were recorded. Ventricular tachycardia was defined as 4 or more consecutive ventricular premature beats⁸⁵. VF was defined as a signal where individual QRS deflections could not easily be distinguished from each other and where rate could no longer be measured⁸⁵ Episodes referred to the number of episodes of VT or VF. The duration of each episode was recorded in seconds and the sums of these were analysed. To overcome the occasional difficulty of identifying VT or VF, the frequency and duration of both were summed and analysed separately. For example, a VT with torsade de pointes morphology that converted to VF then reverted to VT without a clear-cut interface was included in the summed measurement⁸⁴ Notwithstanding this limitation, every attempt was made to identify VT and VF as separate variables.

Statistical Analysis: All values were expressed as means±SE of the mean. For infarct size data, a one-way analysis of variance (ANOVA) was used with a least significance difference (LSD) post hoc test. A Mann-Whitney U test was used for comparison of arrhythmia frequency and duration because the variables of VT and VF are not normally distributed⁸⁴ Hemodynamic data (heart rate, mean arterial pressure and rate-pressure product) was compared using an ANOVA for repeated measures. Statistical significance was defined as a P value of ≦0.05.

Three rats were excluded from the study: one animal's MAP was <70 mmHg before treatment (Lido-only), a second animal's ventilation tubing became clogged (AL solution group), and a third rat from Lido-only group died before the end of the experiment from severe hypotension; no ventricular arrhythmias were involved. Data from a total of 52 rats is reported and the mean body weight was 361±3 g. No significant differences in rat weights were found between the groups.

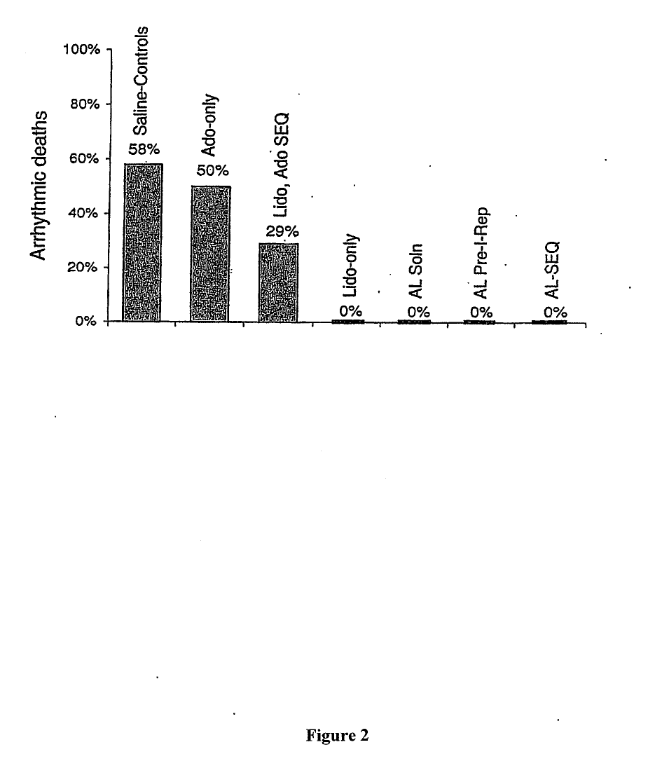

For Study I described above, mortality data are summarised in FIG. 2. Seven of the 12 (58%) saline-control rats and 4 of the 8 (50%) Ado-only treated rats died during the ischaemic period from an episode of ventricular fibrillation. No deaths occurred in the Lido-only treated rats (n=6) or in AL solution infused animals (n=7) (FIG. 2). Only data from surviving rats were further analysed.

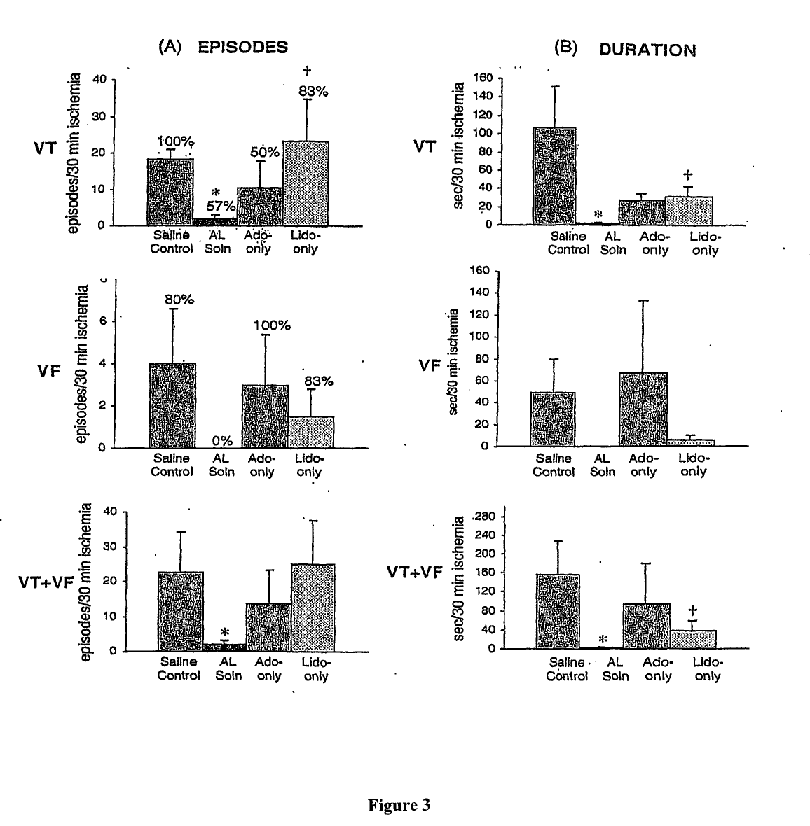

The mean number of episodes of ischaemia-induced VT in saline-controls was 18±9 affecting 100% of animals (FIG. 3a), and 40% experienced VF (4±3 episodes). Treatment with Ado-only resulted in VT in 50% of the rats tested (11±7 episodes) and 100% of rats had VF (3±2 episodes). In Lido-only treatment, ventricular tachycardia occurred in 83% (23±11 episodes) and VF in 33% (2±1 episodes) of rats tested. In AL solution treated rats, 57% of subjects had at least 1 episode of VT (2±1) while no rats experienced a single episode of VF (FIG. 3a).

There were no significant differences in duration of arrhythmias in the Ado-only or Lido-only treatments compared to saline-controls, or to each other (FIG. 3b). Rats infused with AL solution experienced not only a significant reduction in VT's, but also a significant reduction in durations of VT (2±1 seq) and VT+VF's (2±1 sec) compared to saline-controls. The durations of VT and VT+VF's for saline-controls were 106±45 seq and 156±72 seq and for Lido-only treatment were 31±18 sec and 37±22 see respectively (FIG. 3b). In addition, infusion of AL solution significantly reduced the durations of the VT episodes compared to Ado-only treated rats (27±18 sec) (FIG. 3b). It was noted that, with the exception of the AL solution group, a high variability in arrhythmia frequency and duration across treatment groups was observed (FIG. 3). Only the infusion of AL solution provided consistent reductions of VT or VF frequency or duration without large variability between samples.

In respect of reperfusion arrhythmias, within the first minute of reperfusion, 80% of saline-controls, 75% of the Ado-only and 16% of Lido-only treated animals experienced at least one episode of VT of 0.6 to 35 seq duration. Neither treatment with Ado-only or Lido-only differed significantly from each other, or from saline-controls (P<0.05). An episode of VF occurred in 1 of the 5 saline-controls within the first minute and lasted 16 sec. There were no episodes of VF in Ado-only or Lido-only treatment groups during 30 min reperfusion. Rats treated with AL solution experienced no ventricular arrhythmias (VT or VF) at or during reperfusion. The number of episodes of VT from saline-controls and the Ado-only treated animals was found to be significantly higher than AL solution treated animals. Additionally, the durations of VT and VT+VF durations in the Ado-only group (11±8 seq for both) were significantly longer than treatment with AL solution.

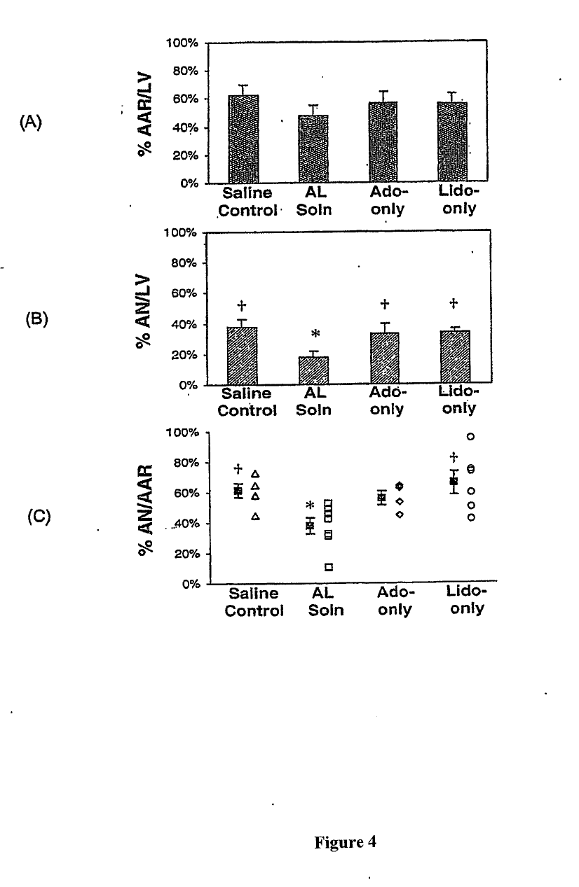

Mean area at risk as a proportion of the left ventricle (AAR/LV), areas of necrosis (AN/LV) and infarct size (AN/AAR) expressed as a percentage of left ventricle are shown in FIG. 4 (a) to (c). The areas at risk for saline-controls, Ado-only, Lido-only and AL solution treated animals were 63±7%, 58±8%, 56±8% and 48±8% respectively, and not significantly different (P<0.05). Overall, the mean risk area was 55±4% (n=22) (FIG. 4a). The areas of necrosis for saline-controls, Ado-only and Lido-only animals were 38±5%, 33±7% and 33±3% respectively, and were not significantly different from each other (FIG. 4b). In contrast, the area of necrosis in AL solution treated animals was 18±4% and significantly lower all other treatments (FIG. 4b). Similarly, the mean infarct size was reduced in rats infused with AL solution (38±6%) compared to saline-controls (61±5%) (P<0.05), Lido-only treated animals (66±8%) (P<0.05), and the Ado-only group (56±5%) (P=0.06) (FIG. 4c). There was no significant difference between mean infarct sizes between saline-controls, Ado-only, or Lido-only treatments.

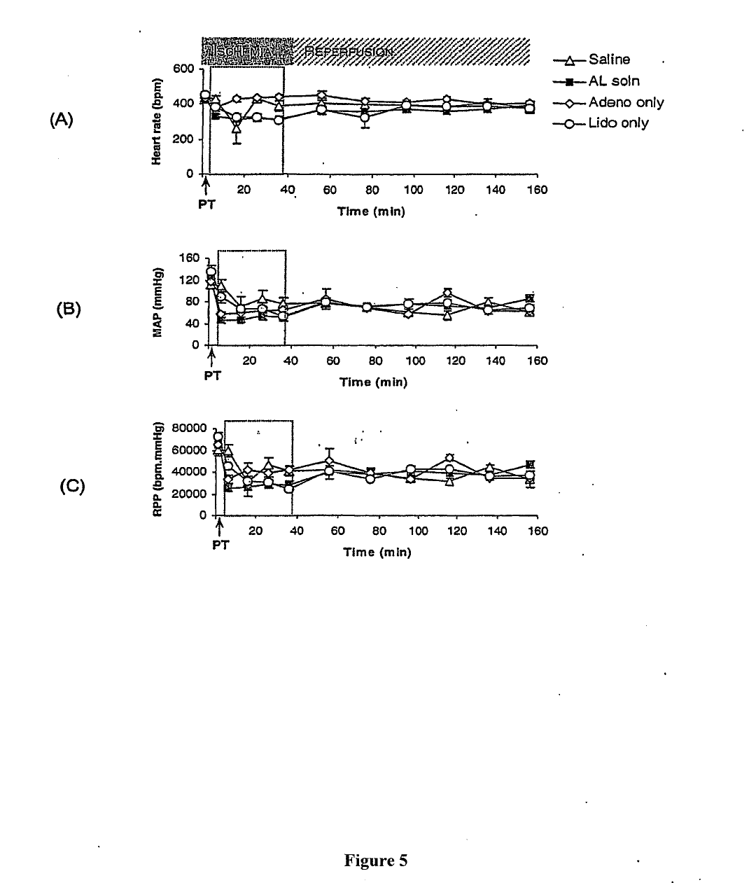

Heart rate (HR), mean arterial pressure (MAP) and rate-pressure product (RPP) are shown in FIG. 5 (a) to (c), respectively. There were no significant differences among groups prior to pretreatment. Pretreatment of either AL solution or Ado-only resulted in equivalent decline in MAP and RPP while MAP and RPP of saline-controls and Lido-only treated animals were similarly elevated. Both Lido-only and AL solution resulted in bradycardia while Ado-only and saline treatment maintained heart rate throughout ischaemia. The dramatic drop in heart rate shown at 10 min ischaemia in the saline-control group was associated with ventricular fibrillation during that time (FIG. 5a). Otherwise, heart rate was sustained in saline-controls. Although AL solution treatment resulted in decreased hemodynamics throughout ischaemia, the decline of both MAP and RPP was not statistically different from other treatments. Only at the end of 30 min ischaemia did the RPP between treatments diverge. Saline-controls and Ado-only treatment rose to levels statistically higher than AL solution and Lido-only treatment.

At reperfusion, coinciding with the discontinuation of treatment, hemodynamics in all groups rose toward pretreatment values. However, within the 120 min reperfusion period, no treatment reached starting baseline values in any group. Despite this, AL solution treatment resulted a significant improvement in MAP by the end of 120 min reperfusion compared to all treatment groups.

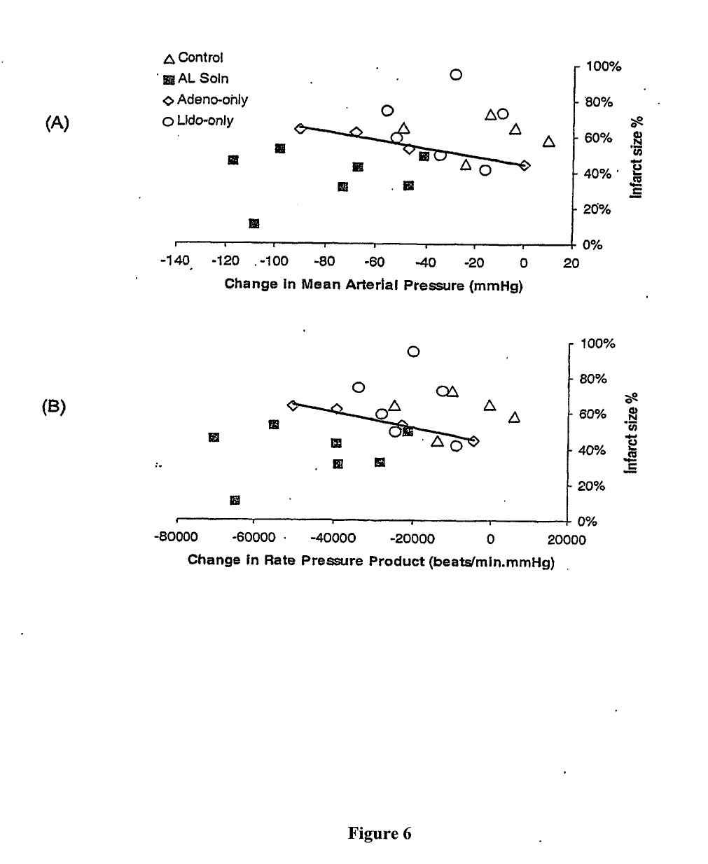

Evaluation of hemodynamics from all groups indicated no correlation between infarct size and MAP or RPP at pretreatment or assessed every 5 min during 30 min ischaemia. However, to ensure that individual treatments' hemodynamic changes did not lead to reduced infarct sizes instead of the treatment itself, a correlation analysis was performed on individual group MAP and RPP changes following pretreatment (FIGS. 6a and 6b). Hemodynamic changes from saline-control and the Lido-only pretreatment did not significantly affect infarct size. Treatment with Ado-only resulted in a correlation between the reduction in hemodynamics from pretreatment and infarct size. The more Ado-only treatment reduced MAP or RPP, then the greater the infarct size (MAP, R²=0.96, p=0.020; RPP, R²=0.98, p=0.012). Pretreatment with AL solution led to infarct size reduction which was independent of changes in MAP or RPP (p≧ 0.60), despite the dramatic decrease in all hemodynamic variables accompanying pretreatment with AL solution.

The applicant found that a greater decrease in MAP (R²=0.96, p=0.020) and RPP(R²=0.98, p=0.012) correlated with a larger infarct size. There was no significant effect on infarct size by hemodynamic changes in either the saline control groups, AL solution or the Lido-only pre-treatment.

While Lido-only and AL solution treatment led to similar RPP and MAP responses throughout ischaemia (FIG. 5), the effect of these treatments on infarct size were opposing. Treatment with AL solution decreased infarct size by nearly 42% from controls while Lido-only treatment resulted in an infarct size increase of about 8% above saline-controls (FIG. 4), yet both groups showed no significant differences in hemodynamic properties during ischaemia.

The results of Study II directed to the effect of sequential administration of AL solution or adenosine and lidocaine during ischaemia and/or reperfusion follow. Mortality data is summarised in FIG. 2. Pretreatment with a 2 mg/kg lidocaine bolus resulted in two deaths from ventricular fibrillation during ischaemia before adenosine infusion commenced (Lido, Ado SEQ, n=7). In contrast, no deaths occurred from ischaemia-induced arrhythmias in rats pretreated with 5 min of AL infusion, which was resumed for 5 min before reperfusion and continued during 30 min reperfusion (AL SEQ) (see FIGS. 1 and 2). Similarly no deaths were recorded in animals continuously infused with AL for 5 min pretreatment, 30 min ischaemia and 30 min reperfusion (AL-Pre-I-Rep) (FIG. 1).

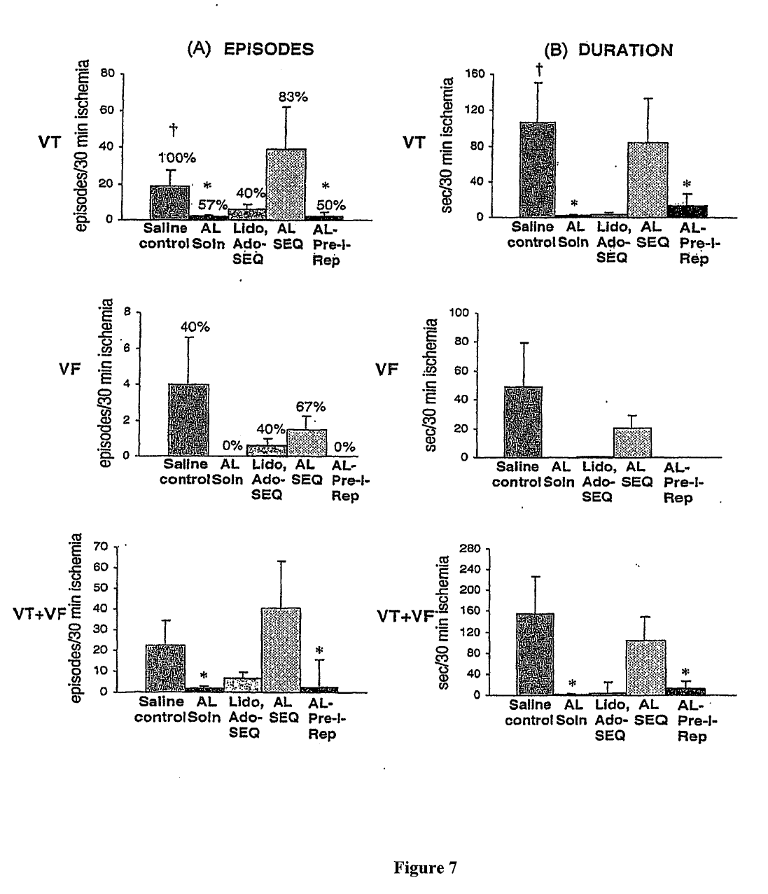

The episodes and durations of VT and VF from rats that survived ischaemia are shown in FIG. 7. Forty percent of the lidocaine-pretreatment group (Lido, Ado SEQ) experienced 6±3 episodes of VT of 4±2 sec duration and 1±0 episodes of VF of 1±0 sec duration during ischaemia (before adenosine infusion) (FIG. 7). The sum of VT and VF episodes and durations for these groups were 7±3 and 4±2 sec respectively. The lidocaine pretreatment strategy (Lido, Ado SEQ) did not significantly reduce episodes or durations of VT or VF compared to saline-controls. In contrast, animals infused with AL during 5 min pretreatment and continued throughout 30 min ischaemia and reperfusion experienced significantly reduced episodes and durations of VT (2±1, 2±1 sec, 57% affected) and VT+VF (2±1, 2±1 sec) compared to saline-controls (18±9, 106±45 sec, 100% affected and 22±12, 156±72 sec, respectively) (P<0.05). However, a 5 min pretreatment of AL solution discontinued during ischaemia (AL SEQ) was not sufficient to prevent VF episodes (2±1, 21±8 sec, 67% affected), or reduce VT (39±23, 84±49 sec, 83% affected) and VT+VF (40±23, 104±46 sec). Importantly, only constant infusion of AL solution before and during ischaemia prevented episodes of VF during ischaemia.

Animals pretreated with a 2 mg/kg bolus of lidocaine followed by another lidocaine bolus and adenosine infusion (Lido, Ado SEQ) before reperfusion experienced 6±3 arrhythmia episodes of 7±4 sec duration at reperfusion (48% VT and 52% VF). Neither the number of VT+VF episodes nor the durations of these were significantly different from saline-controls (P<0.05). In contrast, AL given as pretreatment and for 30 min ischaemia resulted in significantly fewer early reperfusion-induced arrhythmias than the separate and sequential infusions of lidocaine and adenosine (Lido, Ado SEQ). Likewise, there were no reperfusion-induced arrhythmias in animals given AL solution in any sequence (AL SEQ, AL-Pre-I-Rep) resulted in significantly reduced episodes in comparison to saline-controls (P<0.05).

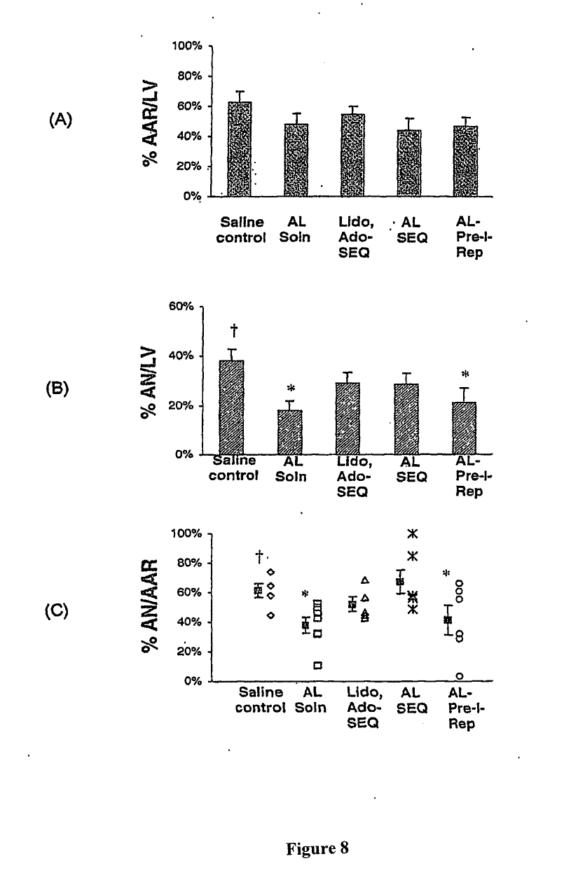

Mean area at risk, areas of necrosis and infarct size (as for Study I) are shown in FIG. 8. The areas at risk for lidocaine-bolus/adenosine infusion (Lido, Ado SEQ), sequential AL infusion (AL SEQ) and constant AL infusion (AL-Pre-I-Rep) were 55±5%, 44±8% and 47±6% respectively. No significant differences were found in risk areas among the different treatment groups and controls (63±7%) (FIG. 8). The area of necrosis for Lido, Ado SEQ (29±4%) and AL SEQ (29±5%) were not significantly different from saline-controls. In contrast, pretreatment with AL solution continued through ischaemia and reperfusion (AL-Pre-1-Rep) significantly reduced left ventricular necrosis (21±6%) compared to controls (38±5%) (FIG. 8). The mean infarct size for Lido, Ado SEQ, AL SEQ and AL-Pre-1-Rep treated groups were 52±5%, 67±8%, and 41±10% respectively and not significantly different from saline-controls (61±5%) (P<0.05). In contrast, when AL was infused continuously for 5 min before and during 30 min ischaemia (results in first study), significant reductions in infarct size (38±6%) were found when compared to either AL SEQ or saline-controls (P<0.05) (FIG. 8).

Heart rate, MAP and RPP at the end of equilibration, after 5 min pretreatment, at 25 minutes ischaemia, 30 minutes reperfusion, 60 minutes reperfusion and 119 minutes reperfusion for the second experiment were analysed. There were no significant differences among groups prior to any treatment. At pretreatment little change occurred to the hemodynamics in the saline-controls and lidocaine-bolus treatment group (Lido, Ado SEQ). Animals receiving the two different AL protocols (AL SEQ, AL-Pre-I-Rep) experienced a significant reduction in all hemodynamic variables at pretreatment, and also at 25 min ischaemia compared to saline-controls or Lido, Ado SEQ group (P<0.05), By 119 min reperfusion MAP was significantly improved in the Lido, Ado-I/R SEQ (86±10 mmHg) compared with groups where AL solution was given for 30 min reperfusion (AL SEQ 66±5, and AL Pre-I-Rep, 69±3) (P<0.05).

Collectively, these results show the effects of adenosine and lidocaine continually infused either individually or combined in solution during ischaemia in an in vivo rat model of regional myocardial ischaemia. In particular, an intravenous infusion of adenosine and lidocaine solution before and during ischaemia offers superior protection from death, arrhythmias and tissue necrosis than either drug alone or when lidocaine bolus preceded adenosine infusion. Further, the sequential administration of lidocaine followed by adenosine during ischaemia and/or reperfusion was inferior to administration of the AL solution as pretreatment and throughout ischaemia, as measured by protection from mortality, arrhythmias and ultimately infarct size.

The infusion of AL solution resulted in no deaths in the four protocols and 26 animals tested (FIGS. 1 and 2). In contrast, 58% of the saline-controls, 50% of the Ado-only treated animals, and 29% of the animals receiving a 2 mg/kg bolus of lidocaine died during ischaemia from ventricular fibrillation (FIGS. 1 and 2). Given adenosine's well-known role to potentiate the abolition of catecholamine triggered ventricular arrhythmias^{87,88}, and the nucleoside's ability to reduce myocardial injury when administered prior to and during regional or global ischaemia^{22,89-92}, it was surprising that the adenosine only infusion failed to protect from death. Adenosine may have either failed to protect the heart from arrhythmias or, based on the higher relative durations of VF compared to durations in saline-controls, may have promoted arrhythmias. The applicant believes that death during ischaemia with adenosine infusion has not been reported before in the rat, dog, pig or human. Thus, these results were unexpected. It seems unlikely that they relate to the concentration administered. Lee et al. infused similar concentrations of adenosine (250-350 ug/kg) for 10 min in humans prior to elective cardiopulmonary bypass surgery without untoward effects⁹³. Indeed, it was found that adenosine pretreatment improved post-bypass left ventricular function compared to no treatment, and that benefit continued 40 hours postoperatively⁹³. Arrhythmias were not investigated. Higher doses of adenosine have been used in other surgical settings without adverse effects. Lagerkranser et al., used a dose range of 60-350 ug/kg/min i.v. in patients undergoing surgery for cerebral aneurism and found that adenosine-induced hypotension (MAP of 40-50 mmHg) did not affect cerebral oxygenation unfavourably⁹⁴.

In contrast to saline-controls and the Ado-only treatment, rats infused with Lido-only experienced no arrhythmias that resulted in death (FIG. 2). However, when a rapid bolus of lidocaine was given prior to ischaemia, 29% of the animals died despite comparatively low episodes and durations of arrhythmias among surviving animals (FIG. 6). These deaths occurred in the ischaemic period before the second bolus of lidocaine and adenosine infusion commenced (Lido, Ado SEQ group; see FIG. 1). While the early work of Homeister et al.,²⁹ did not study the effect of lidocaine or adenosine on mortality rates, they did exclude 6 dogs that had received a rapid bolus of lidocaine (2 mg/kg i.v.) because of intractable VF, and five saline-controls²⁹. Presumably, these subject exclusions died during ischaemia. In the study, infusion of a lidocaine bolus failed to reduce arrhythmias. The combination of adenosine and lidocaine in AL solution, however, was outstanding among all other treatments in consistently abolishing ventricular fibrillation. Even when AL solution was only applied at pretreatment there were no episodes of death, despite a variable amount of arrhythmias during ischaemia (FIG. 6).

Likewise during reperfusion, rats receiving Ado-only, Lido-only or lidocaine bolus/adenosine infusion group (Lido, Ado SEQ) experienced VT or VF early during reperfusion. Again, rats infused with AL solution experienced no early reperfusion-induced arrhythmias in any of the four protocols and 26 animals tested (see results). Without being bound, the applicant speculates that the genesis of early reperfusion-induced arrhythmias may be related to oxygen-derived free radicals⁹⁵, and that AL solution attenuated the formation of reactive oxygen species such as hydrogen peroxide or free radical generation. While both adenosine and lidocaine alone have been shown to be protective against reactive oxygen species^{96,97}, the separate and sequential infusion of lidocaine and adenosine failed to stop such arrhythmias in the study.

Rats treated with AL solution before and during ischaemia had infarct sizes significantly lower (38±6%) than saline-controls (61±5%), Ado-only (56±5%) and Lido-only treatment (66±8%) (FIG. 4). If AL solution was continued through 30 min reperfusion the infarct size reduction was virtually unchanged (41±10%) compared to AL infusion during pretreatment and 30 min ischaemia (38±6%) (FIG. 4). When AL solution was administered only at pretreatment and then again 5 min before reperfusion, no deaths occurred, but the mean infarct size was larger (67±8%) than saline-controls. These results support the conclusion that, for infarct size reduction, AL solution must be applied at least at pretreatment and throughout the ischaemic period. Moreover, when lidocaine was given as a pretreatment bolus followed by another bolus and adenosine infusion prior to reperfusion (Lido, Ado SEQ), the infarct size was high (52±5%), again reinforcing the conclusion that sequential treatments of adenosine and lidocaine are not as effective as a constant infusion of a mixture of adenosine and lidocaine in solution.

On the basis of the relationship between aortic diastolic pressure, coronary perfusion pressure, and myocardial oxygen supply, it was expected that any treatment-induced decrease in hemodynamic variables from pretreatment and throughout ischaemia would correlate with infarct size reduction. However, the study failed to show a statistical difference between a decrease in MAP or RPP and reduced infarct size. Indeed, treatment with adenosine only resulted in the reverse: higher infarct sizes were associated with lower MAP and RPP. Its arguable that the limited number of samples that survived for infarct sizing in the Ado-only group may have reduced the power of that data; however, a large percentage of the animals given Ado-only died from VF during ischaemia during treatment. Overall, this indicates that adenosine alone was incapable of protecting the myocardium, regardless of the limited number of infarct sizes that could be assessed.

On the whole, a reduction in infarct size was observed only in the AL solution group (FIG. 4). Particularly interesting, the Lido-only and AL solution treatment groups incurred similar MAP and RPP during ischaemia, yet infarct size outcomes were the most widely separated. The infarct sizes resulting from ischaemia were more greatly reduced in the AL solution than from Lido-only treatment, which appeared to not protect from infarction. Similarly, sequential administration of AL (AL SEQ), infused once at pretreatment and then again just before reperfusion, resulted in reduced hemodynamics similar to AL solution. However, the data clearly showed that protection from arrhythmias and infarct expansion was not achieved without continuing AL treatment during ischaemia. Therefore, lowering demand or work on the heart with AL solution did not appear to play a role in reducing infarct size or arrhythmias in this study.

Without being bound by any theory or mode of action, it is believed that protection is related to the synergistic effect of adenosine and lidocaine combined to reduce calcium entry into the myocardial cell. A mechanistic synergy between adenosine and lidocaine action may occur that affords the myocardium protection. This data imply that each drug amplifies the effect of the other leading to a reduction in infarct size, episodes of ventricular arrhythmias and death compared to the administration of either drug alone. For example, it is known that Ca²⁺ overload in the ischaemic myocardium predisposes the tissue to injury in part by disturbing membrane linked ionic homeostasis and maintenance of the membrane potential which can lead to high incidences of arrhythmias^{98,99} Reducing intracellular Ca²⁺ overload is likely due to a complex interaction between adenosine and lidocaine targets involving the opening the A1-mediated ATP-sensitive potassium channels (K_{ATP} channels)²² whilst blocking sodium (Na⁺) channels having the overall effect of reducing Na⁺ entry and the activity of Na⁺/Ca²⁺ exchanger^{100,101}. In addition, these actions may enhance cAMP-linked attenuation of VT¹⁰². Furthermore, that no reperfusion arrhythmias were found in any of the AL solution treated rats, demonstrates that protection extended into the reperfusion period. Yoshida et al¹⁰³ have shown in humans that reperfusion VT are most likely arrhythmias triggered by cAMP mediation rather than re-entrant electrical circuits. Whereas Lu et al.¹⁰⁰ have attributed inhibition of Ca²⁺ loading by lidocaine's blocking Na⁺ entry which appears more prominent in ischaemic tissue thereby synchronizing myocardial cells and making reentrant arrhythmias less likely. Therefore, the AL solution in the study may have provided a primary window to reduce triggered (adenosine) and re-entrant (lidocaine) arrhythmias through an amplified reduction of cytosolic Ca²⁺ during ischaemia-reperfusion.

AL cardioprotection may also relate to the collective action of both drugs in reducing the inflammation response to injury. Adenosine is a potent modulator of the anti-inflammatory response by strongly inhibiting the activation of neutrophils, platelets and mononuclear leukocytes, which can lead to cytoxicity and endothelial dysfunction^{22, 104-106}. Additionally, Zhao et al.¹⁰⁷ have linked adenosine infusion at reperfusion with reduced PMN accumulation and reduced myocardial apoptosis. Recent work by Nakamura et al.¹⁰⁸ corroborated this finding in rat hearts by showing that PMN accumulation was significantly correlated with the number of apoptotic cells. Lidocaine also modulates a Na-channel independent inflammatory response by inhibiting the priming of human neutrophils and superoxide anion production with a suspected target site in a G_{q}-coupled signalling pathway^{109,110}. Additionally, lidocaine inhibits intracellularly coupled lysophosphatidic acid (LPA) signalling⁶⁹. LPA is an intercellular phospholipid mediator with multiple actions linked to stimulation of inflammatory events such as platelet aggregation and neutrophil activation. As these events are related to the development of anatomic no reflow, AL solution may play a part initially reducing functional damage from ischaemic injury and hinder the progression of anatomic no reflow¹¹¹⁻¹¹³. The effects of AL combination to reduce ischaemia-reperfusion injury may also be linked to reducing the adverse effects of the inflammatory process which includes attenuating the production of free radicals, reducing capillary plugging and minimising direct injury to cardiomyocytes.

Accordingly, AL solution administered 5 min before and during 30 min regional ischaemia resulted in no deaths, lower episode of ventricular arrhythmias and lower infarct size in the in vivo rat model of regional ischaemia. The cardioprotective properties of AL solution during ischaemia and reperfusion may involve opening the A1 receptor-linked K_{ATP} channels, blocking the Na²⁺ fast channels, adenosine and lidocaine's combined effect on cAMP mediated attenuation of ventricular arrhythmias, and suppression of the inflammatory response to injury. Focusing primarily on a pharmacological therapy for reperfusion injury may deny the underlying cause of the injury and its effective treatment. While minimizing reperfusion injury with adenosine has been a focus in recent years, treatment with AL solution before and during ischaemia reinforces the concept that ischaemia and reperfusion are composite events requiring an integrated strategy to optimize protection of an organ or tissue.

It is known that rat studies have differences from clinical scenarios because of differences in mass-specific metabolic rate¹¹⁴, differences in electrophysiological properties¹¹⁵ and functional morphology such as collateral circulation^{116 117}. There is substantial data as to how such translation studies apply to the clinic. Because of the higher metabolic rate in the rat and the extremely short half-life of adenosine (8 sec)⁵², the applicant chose upper range adenosine concentrations that have led to improved function or reduced necrosis in animal models^{35,118} as well as provided a therapeutic benefit to humans^{93,94}. The main problem limiting adenosine's use in humans is its hypotensive effect but this concern can be minimized during surgical procedures or in the clinical setting when adenosine can be administered as an intracoronary bolus or infusion¹¹⁹. In humans, intracoronary infusions of up to 240 μg/min adenosine causes minimal decrease in arterial pressure, heart rate or electrocardiographic variables¹¹⁹. Similarly, intracarotid injections of adenosine of 1000 μg/ml in baboons has a profound effect to increase cerebral blood flow without any significant systemic side effects¹²⁰. In relation to lidocaine, the maximum safe dose of lidocaine for humans is approximately 4 mg/kg i.v. (without epinephrine) and 7 mg/kg i.v. (with epinephrine). Lidocaine also has a short plasma half-life of approximately 8 minutes. Overall, a 70 kg adult should not receive more than around 300-500 mg cumulative dose of lidocaine. For convenience, the example of the invention given above omitted the standard rapid bolus of lidocaine (1-2 mg/kg) that usually precedes a continuous infusion^{29-31,121} and opted for a lower dose (608 μg/kg/ml/min) continuous infusion. Additionally, using lidocaine this way was intended to avoid the reported pro-arrhythmic effects of lidocaine¹²². Another precaution in comparing data on rats and humans are differences in collateral circulation of the heart. However, since humans have a greater collateral circulation than the rat¹¹⁷, superior cardioprotection by AL infusion is expected to have a greater effect in human patients.

### Example 2

### The Effect of the Pharmacological Preconditioning the Heart: Targeting Adenosine Receptors and Voltage-Sensitive Na⁺ Fast Channels

This example investigates the preconditioning effect of combinatorial therapy targeting adenosine receptors and voltage-dependent sodium fast channels in the in situ rat model of regional ischaemia. Adenosine and/or A1 receptor agonist (CCPA) plus lidocaine was co-administered 5 min before and during 30 minutes coronary artery ligation, and the results compared to classical ischaemic preconditioning. Adenosine and lidocaine is used in example 1 as the sole arresting and protecting combination in cardioplegia, and that co-administration of the two drugs at non-arresting concentrations during ischaemia result in better cardioprotection. Cardiac Na⁺ channels initiate and propagate action potentials in the atria, ventricles and intercalated discs, and their gating is believed to rely exclusively on changes in the resting membrane potential^{123,124}. During ischemia, reduced excitability leads to a rise in extracellular K⁺, a less negative membrane potential and a decreased inward Na⁺ current (I_{Na}) which in turn shortens the epicardial action potential duration, reduced Ca²⁺ entry and helps to protect the heart from arrhythmias.

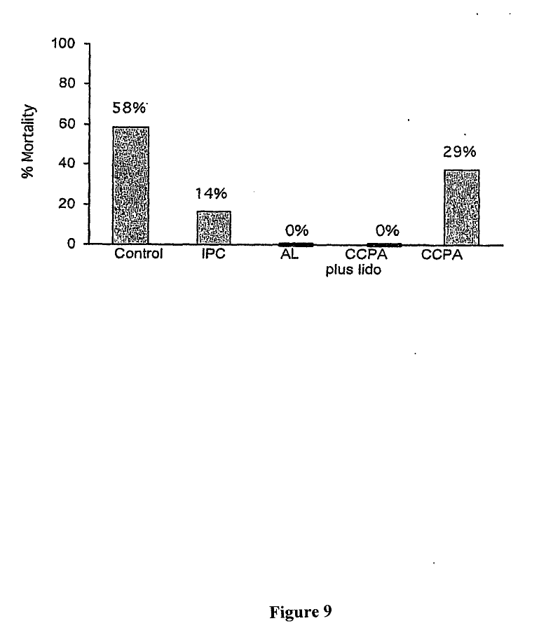

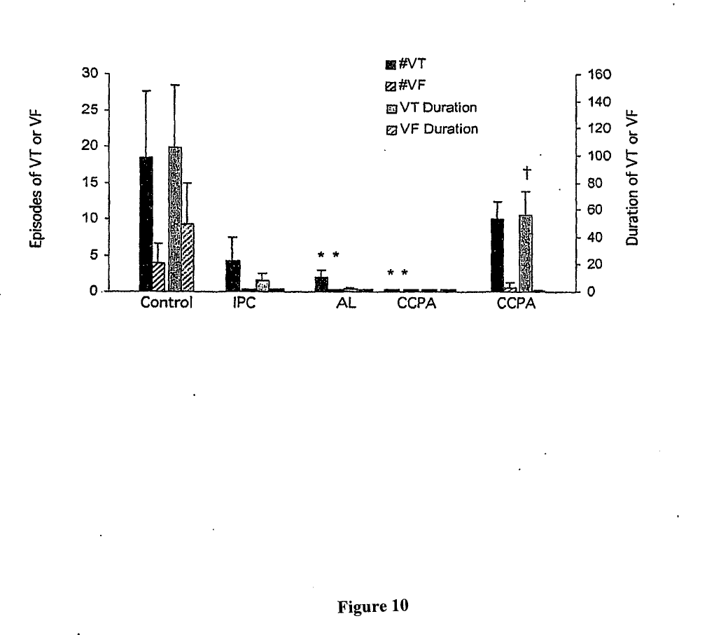

Methods: Rats (n=38) were randomly assigned to one of five groups: (1) Saline controls (0.9% saline) (n=12); (2) IPC (n=6); (3) AL soln (n=7); (4) A1 agonist plus lidocaine (n=6). (5) A1 agonist (CCPA, 5% g/kg) (n=7). Ischaemic preconditioning was achieved using 3 cycles of ischemia/reperfusion with each transition lasting 3 min (Group 2). The adenosine and lidocaine solution (AL soin) was prepared on the day at mass specific dosages of 305 μg/kg/min and 608 μg/kg/min respectively (Group 3). Group 1 and Group 3 rats received continuous infusion of saline or AL soln, respectively, for 5 min before and throughout 30 min of regional ischemia. At the onset of reperfusion the treatment was ceased. Group 4 rats were pretreated 5 min before ligation with a 5 min bolus of A1 agonist CCPA (5 μg/kg) alongside a continuous infusion of lidocaine (608 μg/kg/ml/min) which was continued throughout 30 min ischemia. Group 5 was treated with A1 agonist (CCPA) alone 5 min before ligation. All animals were reperfused for 120 min for infarct sizing. The primary end-points were death, episodes and duration of ventricular arrhythmias and infarct size. Hemodynamics constituted the secondary end-points (heart rate, mean arterial pressure and systolic pressure). Infarct size is considered the “gold standard” of ischaemic preconditioning.
Results: Mortality data are summarized in FIG. 9. Seven of the twelve saline-controls, one of the seven ischaemic preconditioned (IPC) rats and two in the CCPA-treated group (n=8) died during 30 min ischemia from ventricular arrhythmias. In contrast, none of the adenosine and lidocaine-treated rats (n=7) or CCPA plus lidocaine-treated rats (n=6) died (FIG. 10). Only data from surviving rats were further analyzed. Episodes and duration of ventricular tachycardia or fibrillation during 30 min ischemia are shown in FIG. 10. Saline controls had 156±72 sec of ventricular arrhythmias (VT, 106±45; VF, 49±30), and CCPA-treated animals had 56±18 sec of VT with virtually no fibrillation (FIG. 10). Forty percent of the IPC treated-rats experienced 4±3, episodes of VT for over 8±6 sec. Preconditioning with AL abolished VF and significantly reduced episodes and durations of VT with the average duration of the VT 2±1 sec from controls (106±45 sec). Within the AL-treated group, 42% of animals did not experience VT or VF (FIG. 10). Treatment with CCPA plus lidocaine completely abolished VT and VF in all animals tested (FIG. 10). Immediately following ischemia, 80% of saline-controls, 60% of IPC-treated, and 100% of CCPA-treated rats experienced reperfusion tachycardias (Data not shown). No ventricular arrhythmias during reperfusion were experienced in rats preconditioned with AL or CCPA plus lidocaine (FIG. 10).

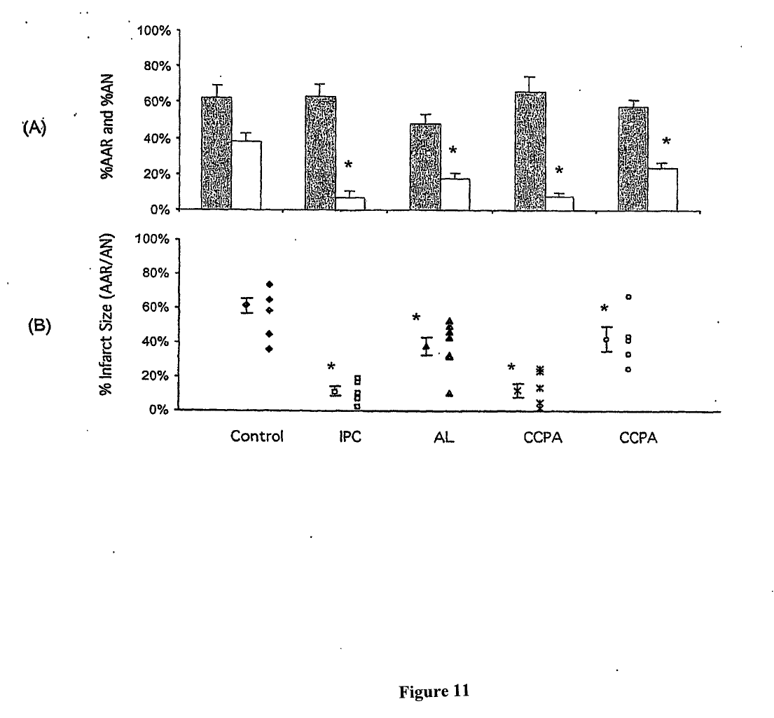

The mean area at risk per left ventricle (AAR/LV), areas of necrosis (AN/LV) and infarct size (AN/AAR) are shown in FIG. 11. The areas at risk expressed as a percent of the left ventricle were not significantly different among the five groups, and on average comprised 58±2% (FIG. 11). The areas of necrosis in saline-controls, AL soln, A1 agonist (CCPA) alone, IPC and A1 plus lido-treated rats were 38±5%, 18±4%, 24±3%, 7±2% and 8±3%, respectively. These measurements translated into a mean infarct size 61±5% for saline-controls, 38±6% for AL soln treated rats, 42±7% for A1 agonist (CCPA) treated animals, 11±3% for IPC treated animals and 12 for CCPA and lidocaine-treated rats (FIG. 11). IPC and pharmacological preconditioning with CCPA and lidocaine-treated rats were not significantly different (P<0.05) (FIG. 11).

TABLE 1
Heart rate and mean arterial blood pressure

| | | | Ischemia | 30 min | 120 min |
|---|---|---|---|---|---|
| Treatment | | Baseline | Start | Ischemia | Reperfusion |
| Saline- | HR (bpm) | 436 ± 13 | 433 ± 15 | 391 ± 31 | 381 ± 30 |
| controls | MAP (mmHg) | 112 ± 6 | 110 ± 11 | 77 ± 11 | 62 ± 8 |
| | Systolic | 139 ± 6 | 137 ± 11 | 104 ± 6 | 86 ± 12 |
| | (mmHg) | | | | |
| IPC | HR (bpm) | 438 ± 9 | 416 ± 16 | 414 ± 7 | 379 ± 8 |
| | MAP (mmHg) | 130 ± 8 | 90 ± 19 | 92 ± 14 | 69 ± 6 |
| | Systolic | 163 ± 12 | 116 ± 22 | 116 ± 14 | 98 ± 6 |
| | (mmHg) | | | | |
| AL soln | HR (bpm) | 497 ± 13 | 332 ± 14*† | 316 ± 17*† | 395 ± 11 |
| | MAP (mmHg) | 123 ± 11 | 46 ± 4*† | 52 ± 6† | 86 ± 6 |
| | Systolic | 159 ± 11 | 75 ± 7*† | 86 ± 8† | 119 ± 7 |
| | (mmHg) | | | | |
| A1 | HR (bpm) | 436 ± 18 | 270 ± 14*† | 172 ± 26*† | 347 ± 17 |
| agonist | MAP (mmHg) | 110 ± 12 | 49 ± 4*† | 44 ± 2*† | 72 ± 5 |
| (CCPA) | Systolic | 131 ± 9 | 77 ± 7*† | 59 ± 3*† | 97 ± 9 |
| plus | (mmHg) | | | | |
| lidocaine | | | | | |
| A1 | HR (bpm) | 421 ± 15 | 336 ± 29*† | 308 ± 73 | 367 ± 12 |
| agonist | MAP (mmHg) | 114 ± 6 | 89 ± 10 | 91 ± 10 | 71 ± 2 |
| (CCPA) | Systolic | 146 ± 6 | 113 ± 11 | 113 ± 12 | 94 ± 4 |
| only | (mmHg) | | | | |

Data are mean ± S.E.M.;
*P < 0.05 vs. control.
†P < 0.05 vs. IPC

The hemodynamic changes during pretreatment, ischemia and reperfusion are found in Table 1. Rats treated with AL or CCPA plus lidocaine had significant reductions in heart rate, MAP and systolic pressure compared to saline-controls and IPC. No significant differences in MAP were apparent between AL or CCPA plus lidocaine, although heart rate was lower in the latter (Table 1). Though all groups' hemodynamic measurements were lower than baseline after 2 hrs reperfusion, no group was significantly different from another.

Ischaemic preconditioning (IPC) remains one of the most potent means of cardioprotection known. Nearly every IPC study has shown a profound reduction in infarct size, and most have reported a large reduction in the incidence of arrhythmias; while others, including the original study of Murry et al.,^{41,125}, have shown that IPC may have a proarrhymic effect and increase the possibility of stunning (Metzner, Yellon). The results in this example demonstrate that pretreating the in situ rat heart with adenosine and lidocaine (AL), or with adenosine A1 agonist (CCPA) and lidocaine, 5 min before and 30 min during acute regional ischaemia, results in no deaths, no lethal arrhythmias and a large decrease in infarct size compared to saline-controls. The most surprising result was that infarct size reduction in CCPA plus lidocaine-treated rats (12±4%) matched that of ischaemic preconditioning (11±3%) demonstrating that the combination of adenosine A1 subtype activation and down-regulation of voltage-dependent Na⁺ fast channels was as effective at reducing infarct size as IPC. Moreover, the combination of A₁L (and AL) surpassed IPC protection in having no deaths and abolishing ventricular arrhythmias (FIGS. 9 and 10).

Without being bound by any theory or mode of action, it is believed that adenosine or adenosine A1 agonists with lidocaine protect the myocardium and coronary microvascular at three levels; electrophysiological, mechanical and metabolic. The results in this example demonstrate that ventricular arrhythmias were significantly reduced. Again, without being bound by any theory or mode of action, this is believed to be due to the combination of the composition according to the invention having improved atrial and ventricular matching of electrical conduction and pump performance. Adenosine activates A1 receptors and thus are considered to be involved in slowing the sinoatrial nodal pacemaker rate (negative chronotropy), delaying atrioventricular (A-V) nodal impulse conduction (negative dromotropy), reducing atrial contractility (negative inotropy), and inhibits the effect of catecholamines (via reduction in cyclic AMP and inhibition of Ca²⁺ influx)^{75,126}. It is believed that Adenosine is 30 times more effective in slowing the conductance of A-V nodal than SA pacemakers¹²⁷, which may be more important to terminate abnormal arrhythmias in combination with lidocaine's ability to reduce the voltage dependent Na⁺ entry and resetting membrane potential to a more polarised voltage (i.e. limit the reduction in ischaemic-induced maximum diastolic potential). Lidocaine's pharmacological effects on electrical conduction and excitability appear to be particularly pronounced during ischemia⁶². Lidocaine binds to the intracellular side of the Na channel near the inactivating gating domains. Improved atrial and ventricular matching may be associated with the combined actions of adenosine and lidocaine to downregulate the heart by shortening action potential duration and reduce contractility which would allow less time available for Ca²⁺ entry via L-type channels, and by increasing the diastolic duration interval which may involve a reduced maximum negative membrane potential reached during diastole, a longer slope of phase 4 depolarisation, and a change to the threshold at which an action potential fires. Membrane hyperpolarisation or the slowing of depolarisation in the presence of AL would effectively reduce Na⁺ and Ca²⁺ entry during ischaemia and protect the cells from arrthymias. Since adenosine receptors and sodium channels are also located in intercalated discs⁸³, reduced membrane excitability may also reduce gap-junction coupling which would further benefit atrial-ventricular matching of conduction and pump performance. A1 activation leads to delayed protection by delaying the rise of intracellular Na⁺ and Ca²⁺. This has been demonstrated in rat myocytes and human cell line (tsA201)¹²⁸. Reduced Na⁺ and Ca²⁺ entry would also decrease axial resistance and improve electrical conduction in ischaemic hearts¹²⁸. Furthermore, the probable reduction of atrial and ventricular myocyte excitability, delayed repolarization and therefore increased refractoriness by adenosine receptor stimulation with lidocaine may be linked with a decrease in re-entrant ventricular arrhythmias, particularly in the highly vulnerable epicardial ischaemic zone.

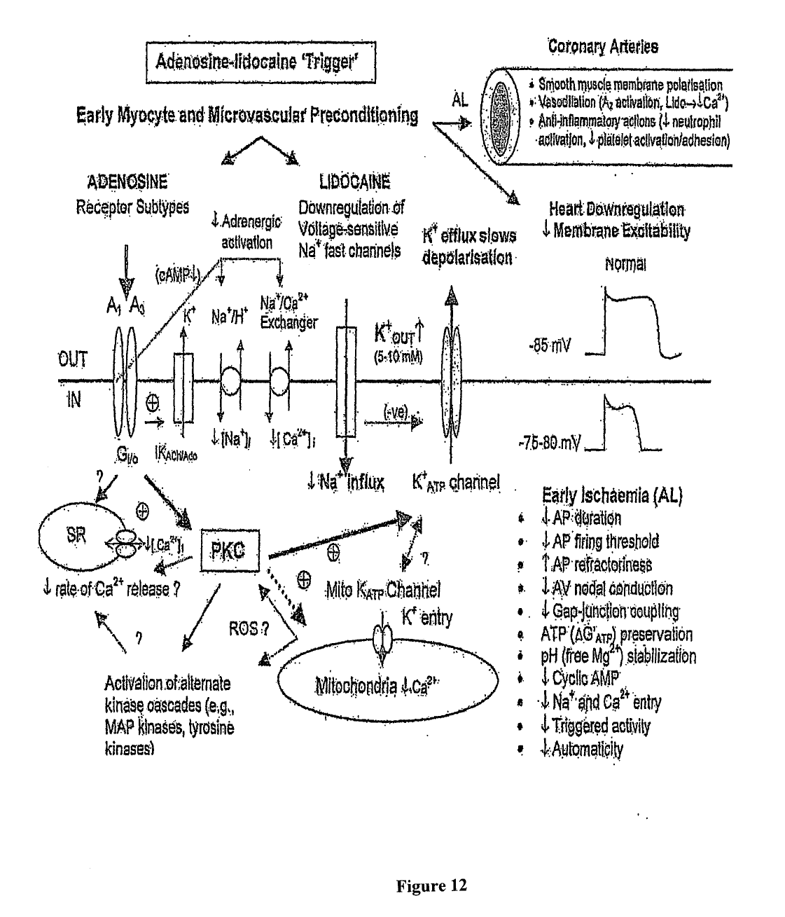

Pretreating the heart with adenosine or A1 agonist with lidocaine resulted in significant cardioprotection as judged by the “gold standard” of infarct size reduction (FIG. 11). Adenosine is thought to be involved in myocardial preconditioning^{36,37}. Adenosine A1 receptor activation (and in some cases A3) has been implicated in the rat³⁹, rabbit^{36,129}, dog¹³⁰, pig¹⁰³ and human^{75,131}. The results are set out in this specification support the role of CCPA A1 activation to reduce infarct size in the rat model (FIG. 11). Adenosine's role as a ‘trigger’ of preconditioning has been supported from studies using the non-selective receptor antagonist 8-(p-sulphophenyl)-theophylline (SPT) which reduces protection a number of animals models^{37,129}. Adenosine A1-receptors, like bradykinin and opioid receptors, are known to confer protection via inhibitory G protein-coupled pathways which have been linked to the opening of sarcolemma ATP sensitive K⁺ channels.¹³² Adenosine A1 receptor ‘trigger’ activation has also been linked to new targets including the mitochondria^{128, 133-135} and sarcoplasmic reticulum.¹³⁶ Nevertheless, it remains to be established how the opening the mitochondrial K_{ATP} channel and/or reactive oxygen species ‘triggers’ and/or mediates the delay of cell injury and how the different K_{ATP} channels relate to one another, and other potential ‘triggers’ to reduce infarct size by preconditioning the heart. Without been bound by any theory or mode of action, FIG. 12 summarise our model of adenosine and lidocaine's possible multiple signalling mechanisms involved in early (classic) preconditioning of the in situ rat myocardium and coronary microvascular.

Lidocaine can reduce acute regional ischemia in heart and brain^{123, 138-140}. Low concentrations of lidocaine bind to amino acids positioned on the intracellular side of the Na⁺ channel near the inactivating gating domains¹⁴¹, and are potentiated by ischaemia¹⁴². The shift in the Na⁺ channel's voltage-dependence to a more polarised state compared to ischaemia alone, and lidocaine's ability to inhibit L-type calcium channels, help explain the drug's anti-ischaemic actions to delay Na⁺ and Ca²⁺ entry into the cell [Haigney, 1994 #1372⁸². Lidocaine's anti-ischaemic effects might also be enhanced by adenosine's anti-adrenergic actions to indirectly inhibit the Na⁺/H⁺¹⁴³ and Na⁺/Ca²⁺ exchangers¹⁴⁴. Thus, due to the central role of voltage-gated Na⁺ channels in modulating Ca²⁺ entry, lidocaine with adenosine or A1 agonist would be expected to delay Na⁺ entry and reduce Ca²⁺ loading. Lidocaine and adenosine also have potent anti-inflammatory properties which without being bound to any theory or mode of action may explain the low number of arrhythmias during ischemia, and particularly in the reperfusion period (FIG. 10). Both adenosine and lidocaine are known to attenuate neutrophil activation^{22,97} and inhibit platelet activation and plugging.^{22,69}

It has been demonstrated that infarct size in AL treated rats falls from 61% to 38%. Since the mean arterial pressure (MAP) was not significantly different between AL and A₁L treatments (Table 1), the contribution of hypotension to infarct-size reduction in the rat model cannot exceed the fall from 61 to 38% (FIG. 3). Thus the infarct size reduction from 38% to 12% in the A₁L treated rats must be due to factors other than hypotension. In this case, the maximal contribution of hypotension to infarct reduction would be 47% [(61−38)/(61−12)×100] in CCPA+L treated rats, with the remaining 53% coming from the pharmacological therapy itself. If hypotension contributed to 50% of the infarct reduction in the AL-treated animals compared to controls, then the direct benefit of the drug combination CCPA+L would be nearly 77%. It thus appears that the direct cardioprotection from A1+L-treated rats is at least 53%. However, it has been shown that hypotension on its own does not reduce infarct size. In 1997 Casati et al., showed in the in vivo rabbit model that the protective action of A1 receptor activation by CCPA was independent of changes to hemodynamics including MAP¹⁴⁵. In this study atenolol (a beta-adrenoceptor blocker), felodipine (a Ca²⁺ channel blocker) and A2A selective agonist (2-hexynyl-5′-N-ethyl-carboxamindoadenosine, 2HE-NECA) and 5′-N-ethyl-carboxamindoadenosine (non-selective adenosine agonist, NECA) reduced MAP similar to CCPA but did not change infarct size¹⁴⁵. In addition, in separate studies the bradycardia effect of CCPA (Table 1) has been shown to contribute little to infarct size reduction. By pacing isolated rat hearts, De Jong and colleagues showed that CCPA was still cardioprotective in paced hearts compared to hearts without pacing. We demonstrate that infarct size reduction in CCPA+L-treated rats is largely due to the combined mechanism of action, not to haemodynamic effects³⁷.

Accordingly, a composition according to the invention has been shown to provide a composition to use as an alternative method to ‘classical’ ischaemic preconditioning involving physical clamping of the heart. The results in this example show that co-administration (i.v.) of the A1 receptor agonist CCPA and Na⁺ fast channel modulator lidocaine 5 min before and during 30 min of left coronary artery ligation results in no deaths, no arrhythmias and a profound reduction in myocardial infarct size which was not significantly different to ischaemic preconditioning. Targeting adenosine A1 receptor subtype and Na⁺ fast channel modulation offers a new therapeutic window to delay myocardial damage during ischemia and improve left contractile function in reperfusion (FIG. 12). In the clinical setting, adenosine-lidocaine preconditioning therapy may be useful in arrhythmia management and could be administered via an intracoronary route for open-heart surgical procedures or for angioplasty where acute systemic hypotension is to be avoided^{42,48}. More importantly this demonstrates that the preconditioned effect of A1 adenosine receptor agonist is not limited to adenosine and lignocaine but can include the other potassium channel openers and/or adenosine receptor agonists, (including indirect adenosine receptor agonists).

### Example 3

### Effect of Adenosine and Lignocaine with Esmolol on Functional Recovery of the Rat Heart after Arrest

This example demonstrates the effect of esmolol, an antiadrenergic, together with Adenosine and Lignocaine on functional recovery after a period of arrest using intermittent perfusion.

Hearts from adult whistler rats (350 g) were prepared using the method described below. Intermittent retrograde perfusion was performed under a constant pressure head of 70 mmHg after hearts were switched back from the working mode to the Lagendorff mode. After stabilisation, the hearts were arrested using either:

(i) Adenosine (200 uM) and Lignocaine (500 uM) plus Esmolol (100 uM);

(ii) Adenosine (200 uM) and Lignocaine (500 uM) plus Esmolol (10 uM);

(iii) Adenosine (20 uM) and Lignocaine (500 uM) plus Esmolol (100 uM).

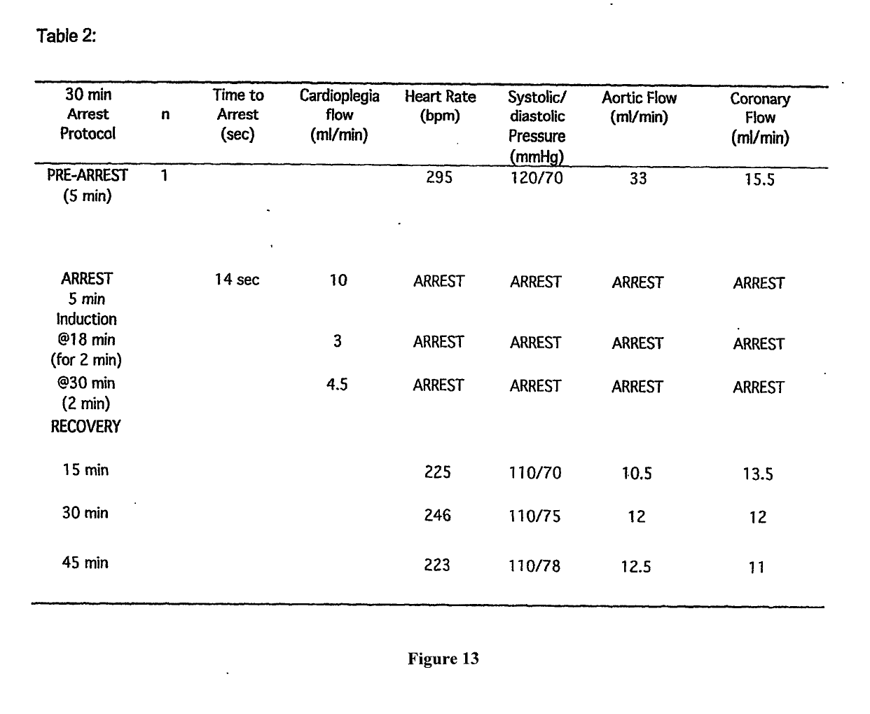

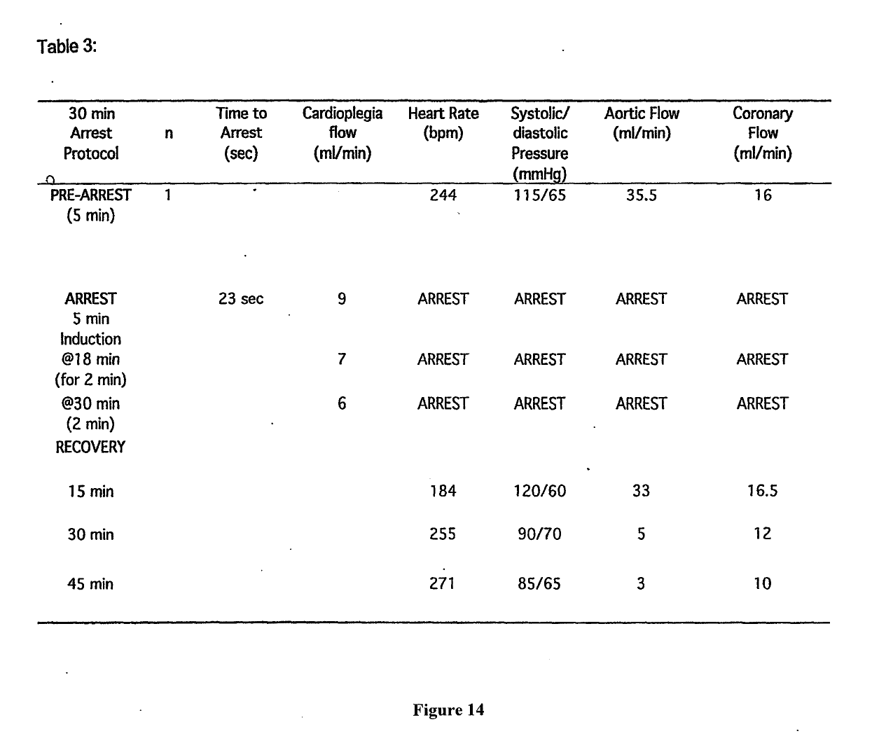

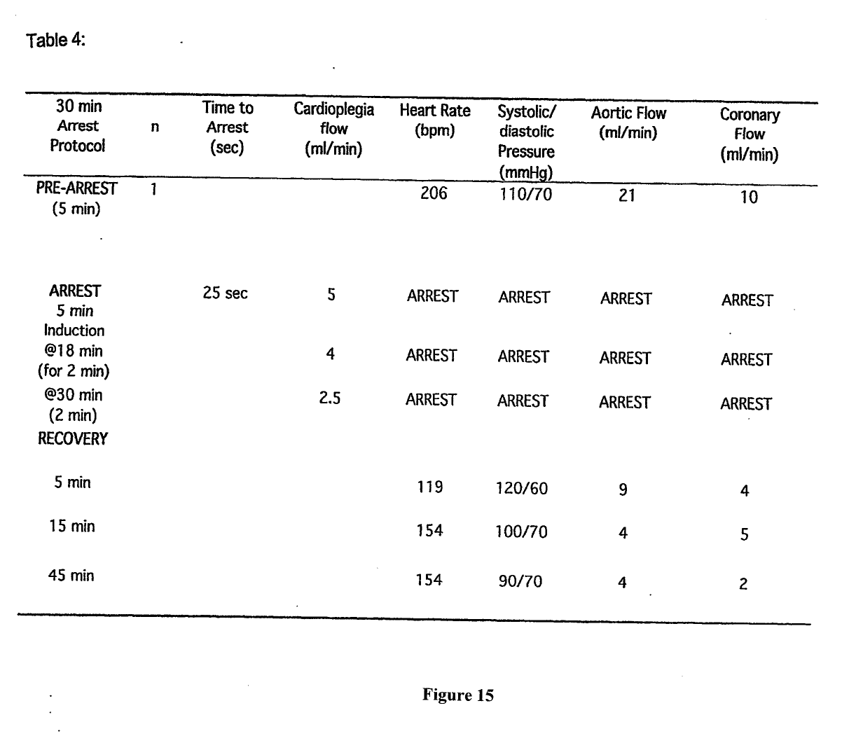

Solutions containing these compounds were provided in Krebs Henseleit (10 nM glucose, pH 7.55 @ 37° C.). The aorta was then cross-clamped and the heart left to sit arrested for 5 mins, after which the clamp was released and 2 mins of arrest solution delivered from a pressure head of 70 mmHg. The clamp was replaced and this procedure continued for 18 mins arrest time then 30 mins arrest time. The recovery results are shown in Table 2 (FIG. 13), Table 3 (FIG. 14) and Table 4 (FIG. 15).

This example demonstrates improved functional recovery of the heart after 30 mins arrest, providing superior protection during arrest and recovery of the heart.

### Example 4

### Effect of Calcium Antagonist Nifedipine in Combination with L or AL to Arrest, Protect and Preserve the Heart

This example investigates the effect of Nifidipine in combination with lidocaine compared to Nifidepine in combination with Lidocaine and Adenosine in arresting protection and preserving the heart. Nifedipine is a Calcium antagonist.

Animals Adult Male Sprague-Dawley rats (˜350 g) were obtained from James Cook University's breeding colony. Animals were fed ad libitum and housed in a 12 hour light/dark cycle. On the day of experiment rats were anaesthetised with an intraperitoneal injection of Nembutal (Sodium Pentabarbitone; mg/kg body wt) and the hearts rapidly excised (details below). At all times animals were treated in accordance with the James Cook University Guidelines for use of ‘Animals for Experimental Purposes’ (Ethics approval number A557). Adenosine (A9251 >99% purity) and all other chemicals were obtained from Sigma Chemical Co (Castle Hill, NSW). Lidocaine hydrochloride was purchased as a 2% solution (ilium) from the local Pharmaceutical Supplies (Lyppard, Queensland).
Krebs-Henseleit Perfusion buffer: Modified Krebs Henseleit buffer contained 10 mM glucose; 117 mM NaCl, 5.9 mM KCl, 25 mM NaHCO₃, 1.2 mM NaH₂PO₄, 1.12 mM CaCl₂ (free Ca²⁺=1.07 mM), 0.512 mM MgCl₂ (free Mg²⁺=0.5 mM), pH 7.4 at 37° C. The perfusion buffer was filtered using a one micron (1 uM) membrane and then bubbled vigorously with 95% O₂/5% CO₂ for a pO₂ above 600 mmHg. The perfusion buffer was not recirculated.
Calcium antagonist nifedipine (RBI N-114. MW 346.34) plus Lidocaine Arrest solution: 0.44 uM nifedipine plus 500 uM lidocaine in 10 mM glucose containing Krebs Henseleit buffer (pH 7.7 at 37° C.). The AL arrest solution was filtered using 0.2 uM filters and maintained at 37° C. The arrest solution was not actively bubbled with 95% O₂/5% CO₂ hence the higher pH (The average pO₂ of the solution was 131 mmHg and pCO₂ of 5-10 mmHg). 0.0035 g Nifedipine was added to 0.5 ml DMSO and 10 ul of this solution was added to 500 ml arrest solution. Final concentration of Nifedipine is 0.44 uM and DMSO 0.002%. Note: uM=micromolar
Calcium antagonist nifedipine (RBI N-114. MW 346.34) plus Adenosine and Lidocaine Arrest solution: 2 uM nifedipine plus 200 uM adenosine plus 500 uM lidocaine in 10 mM glucose containing Krebs Henseleit buffer (pH 7.7 at 37° C.). The AL arrest solution was filtered using 0.2 uM filters and maintained at 37° C. The arrest solution was not actively bubbled with 95% O₂/5% CO₂ hence the higher pH (The average pO₂ of the solution was 131 mmHg and pCO₂ of 5-10 mmHg). 0.0035 g Nifedipine was added to 0.5 ml DMSO and 20 ul of this solution was added to 200 ml arrest solution. Final concentration of Nifedipine is 2 uM and DMSO 0.01%. Note: uM=micromolar
Langendorff and Working Rat Heart preparation: Hearts were rapidly removed from anaesthetised rats and immediately placed in ice-cold Krebs-Henseleit buffer. Excess tissue was removed and the heart was connected via the aorta to a standard Langendorff apparatus with a perfusion pressure of 90 cm H₂O (68 mmHg). After tying off the pulmonary veins and superior and inferior vena cava to minimize leaks (<1 ml/min), the pulmonary artery was cannulated. The preparation was then switched to the working mode and the heart was not placed in a thermostated jacket. The preload was preset at 10 cm H₂O (7.6 mmHg) and the afterload 100 cm H₂O (76 mmHg). Hearts were stabilised for 30 minutes before tying off the coronary artery for 20 min (see below). Heart rate, aortic pressure, coronary flow, aortic flow and oxygen consumption were measured before, during and following the ischaemic injury protocol.

Aortic pressure was measured continuously using a pressure transducer (UFI Instruments, Morro Bay, Calif.) coupled to a MacLab 2e (ADI Instruments). Systolic and diastolic pressures and heart rate were calculated from the pressure trace using the MacLab software. Arterial and venous perfusate pO₂ and pCO₂, pH and ions (Ca²⁺, Cl⁻, and Na⁺) were measured using a Ciba-Corning 865 blood gas machine. Coronary flow and aortic flow were measured in volumetric cylinders. The initial criteria for exclusion of working hearts during the 30 min equilibration period (before ischaemia) was a heart rate less than 200 beats/min, a systolic pressure less than 100 mmHg and coronary flow less than 10 ml/min. No pacing or cardiac massage was employed during the recovery phase in the working mode.

### Mode of Cardioplegic Delivery and Arrest Protocol:

The hearts were then switched to Langendorff mode and 50 ml of cardioplegia was delivered at 37° C. at a constant pressure head of 90 cm H₂O (68 mmHg). For the min arrest protocol, the aorta was cross-clamped for 15 min after which it was released to deliver a 2 min infusion pulse of cardioplegia solution and the clamp reapplied. A terminal cardioplegia infusion was repeated once more at 32 min before the heart was unclamped and switched to working mode at 34 min. Hearts were then returned to working mode and recovery was monitored for 45 to 60 min at 37° C. Protection was assessed by measuring a number of physiological parameters including aortic and coronary flows, heart rate, recovery of systolic and diastolic pressures which were compared to baseline values.

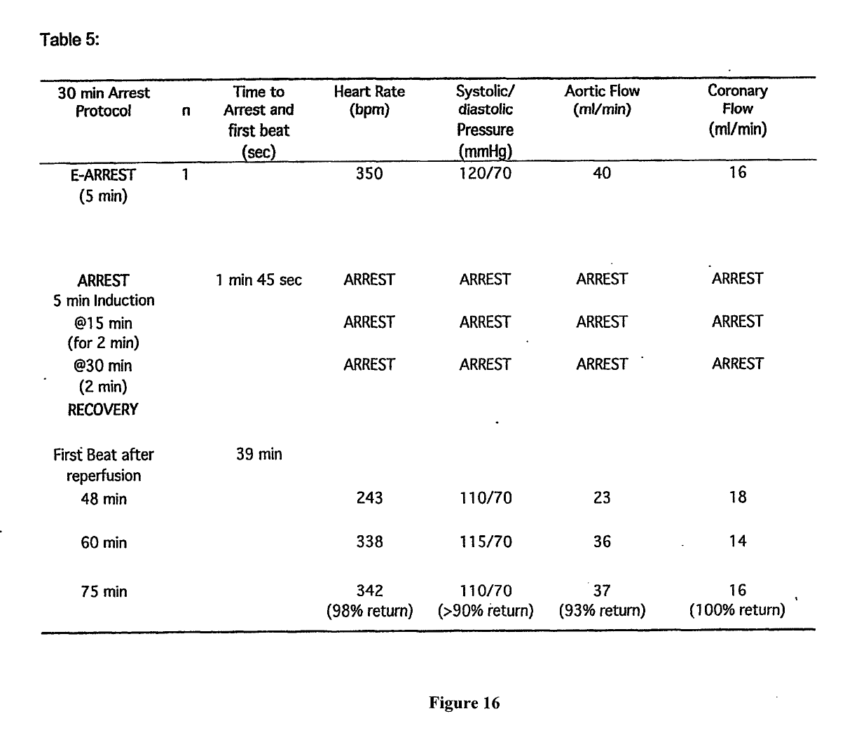

Table 5 (FIG. 16) summarises the results of 0.44 uM nifedipine plus 500 uM lidocaine in 10 mM glucose containing Krebs Henseleit buffer (pH 7.7 at 37° C.). The heart arrested in 1 min and 45 sec and remained arrested throughout the 30 min protocol. Time to first beat after arrest during reperfusion was 39 min. After 48 min heart rate was 69%, pressures were over 90%, aortic flow was 58% and coronary flow was over 100% of pre-arrest values. After 75 min heart rate was 98%, pressures were over 90%, aortic flow was 93% and coronary flow was 100% of pre-arrest values. This example shows that a calcium channel blocker plus a local anaesthetic arrests, protects and preserves the heart.

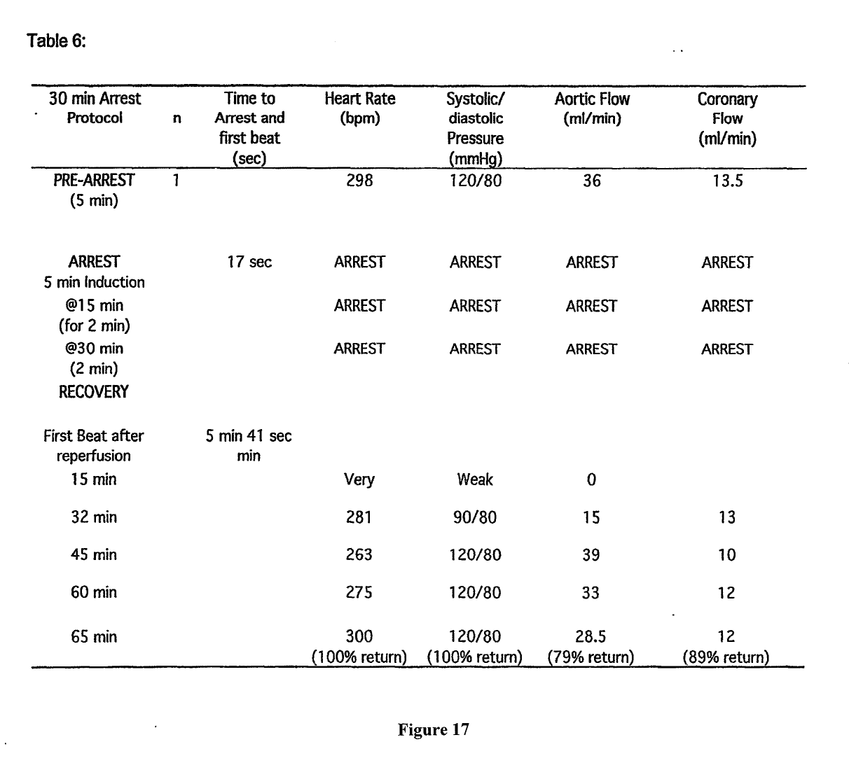

Table 6 (FIG. 17) summarises the results of 2 uM nifedipine plus adenosine (200 uM) and lidocaine (500 uM) in 10 mM glucose containing Krebs Henseleit buffer (pH 7.7 at 37° C.). The heart arrested in 17 sec min and remained arrested throughout the 30 min protocol. Time to first beat after arrest during reperfusion was 5 min and 41 sec. After 15 min the heart was contracting weakly. At 32 min heart rate was 94%, pressures were over 75%, aortic flow was 42% and coronary flow was over 96% of pre-arrest values. After 65 min heart rate was 100%, pressures were over 100%, aortic flow was 79% and coronary flow was 89% of pre-arrest values. This example shows that a calcium channel blocker plus a potassium channel opener or adenosine agonist and a local anaesthetic arrests, protects and preserves the heart.

### Example 5

### Effect of Opioids in Combination with L or AL to Arrest, Protect and Preserve the Heart

Adult mail Sprague-Dawley rats were obtained as per the previous example.

Krebs-Henseleit Perfusion buffer: Modified Krebs Henseleit buffer contained 10 mM glucose; 117 mM NaCl, 5.9 mM KCl, 25 mM NaHCO₃, 1.2 mM NaH₂PO₄, 1.12 mM CaCl₂ (free Ca²⁺=1.07 mM), 0.512 mM MgCl₂ (free Mg²⁺=0.5 mM), pH 7.4 at 37° C. The perfusion buffer was filtered using a one micron (1 uM) membrane and then bubbled vigorously with 95% O₂/5% CO₂ for a pO₂ above 600 mmHg. The perfusion buffer was not recirculated.
Delta-1-Opioid agonist [D-Pen 2,5]enkephalin (DPDPE) plus Lidocaine Arrest solution: 1 uM delta-opioid agonist plus 500 uM lidocaine in 10 mM glucose containing Krebs Henseleit buffer (pH 7.7 at 37° C.). The AL arrest solution was filtered using 0.2 uM filters and maintained at 37° C. The arrest solution was not actively bubbled with 95% O₂/5% CO₂ hence the higher pH (The average pO₂ of the solution was 131 mmHg and pCO₂ of 5-10 mmHg).
Delta-1-Opioid agonist [D-Pen 2,5]enkephalin (DPDPE) plus Adenosine and Lidocaine Arrest solution: 1 uM delta-opioid agonist plus 200 uM adenosine plus 500 uM lidocaine in 10 mM glucose containing Krebs Henseleit buffer (pH 7.7 at 37° C.). The AL arrest solution was filtered using 0.2 uM filters and maintained at 37° C. The arrest solution was not actively bubbled with 95% O₂/5% CO₂ hence the higher pH (The average pO₂ of the solution was 131 mmHg and pCO₂ of 5-10 mmHg).
Langendorff and Working Rat Heart preparation: Hearts were rapidly removed from anaesthetised rats and immediately placed in ice-cold Krebs-Henseleit buffer. Excess tissue was removed and the heart was connected via the aorta to a standard Langendorff apparatus with a perfusion pressure of 90 cm H₂O (68 mmHg). After tying off the pulmonary veins and superior and inferior vena cava to minimize leaks (<1 ml/min), the pulmonary artery was cannulated. The preparation was then switched to the working mode and the heart was not placed in a thermostated jacket. The preload was preset at 10 cm H₂O (7.6 mmHg) and the afterload 100 cm H₂O (76 mmHg). Hearts were stabilised for 30 minutes before tying off the coronary artery for 20 min (see below). Heart rate, aortic pressure, coronary flow, aortic flow and oxygen consumption were measured before, during and following the ischaemic injury protocol.

Aortic pressure was measured continuously using a pressure transducer (UFI Instruments, Morro Bay, Calif.) coupled to a MacLab 2e (ADI Instruments). Systolic and diastolic pressures and heart rate were calculated from the pressure trace using the MacLab software. Arterial and venous perfusate pO₂ and pCO₂, pH and ions (Ca²⁺, Cl⁻, and Na⁺) were measured using a Ciba-Corning 865 blood gas machine. Coronary flow and aortic flow were measured in volumetric cylinders. The initial criteria for exclusion of working hearts during the 30 min equilibration period (before ischaemia) was a heart rate less than 200 beats/min, a systolic pressure less than 100 mmHg and coronary flow less than 10 ml/min. No pacing or cardiac massage was employed during the recovery phase in the working mode.

Mode of Cardioplegic delivery and Arrest Protocol: The hearts were then switched to Langendorff mode and 50 ml of cardioplegia was delivered at 37° C. at a constant pressure head of 90 cm H₂O (68 mmHg). For the 30 min arrest protocol, the aorta was cross-clamped for 15 min after which it was released to deliver a 2 min infusion pulse of cardioplegia solution and the clamp reapplied. A terminal cardioplegia infusion was repeated once more at 32 min before the heart was unclamped and switched to working mode at 34 min. Hearts were then returned to working mode and recovery was monitored for 45 to 60 min at 37° C. Protection was assessed by measuring a number of physiological parameters including aortic and coronary flows, heart rate, recovery of systolic and diastolic pressures which were compared to baseline values.

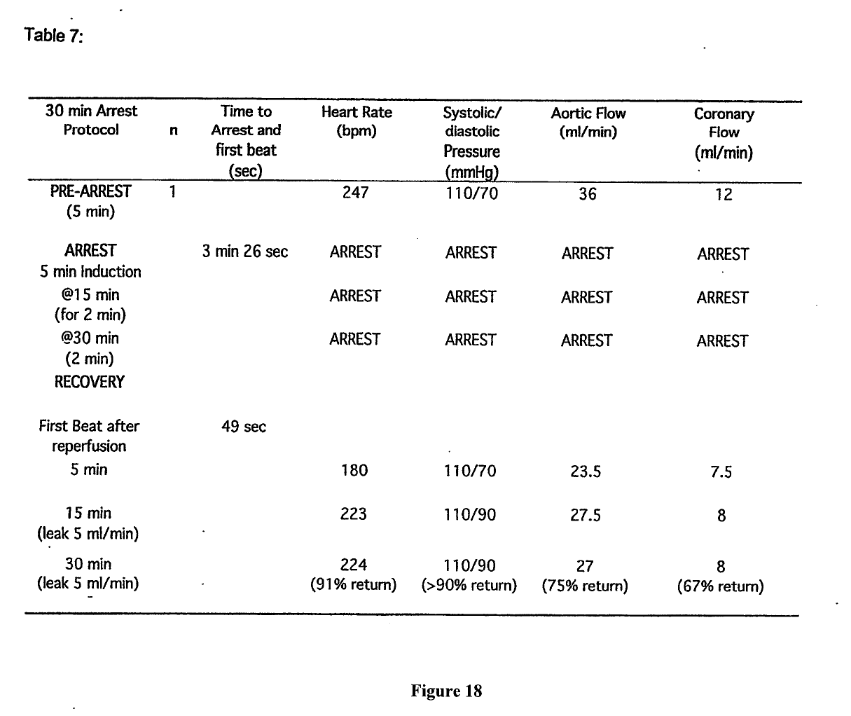

Table 7 (FIG. 18) summarises the results of 2 uM Delta-1-Opioid agonist [D-Pen 2,5]enkephalin (DPDPE) plus 500 uM lidocaine in 10 mM glucose containing Krebs Henseleit buffer (pH 7.7 at 37° C.). The heart arrested in 3 min and 26 sec and remained arrested throughout the 30 min protocol. Time to first beat spontaneously after arrest during reperfusion was 49 sec. After 15 min heart rate was 90%, pressures were 95%, aortic flow was 76% and coronary flow was over 67% of pre-arrest values. After 30 min heart rate was 91%, pressures were over 90%, aortic flow was 75% and coronary flow was 67% of pre-arrest values. This example shows that a delta-1-opioid agonist plus a local anaesthetic arrests, protects and preserved the heart.

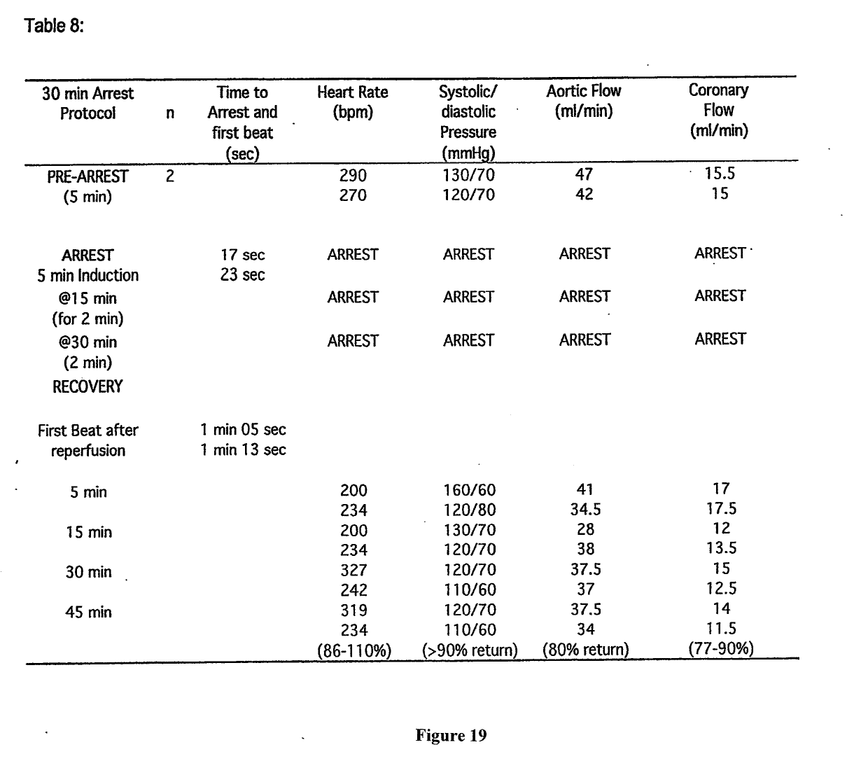

Table 8 (FIG. 19) summarises the results of two hearts receiving 2 uM Delta-1-Opioid agonist [D-Pen 2,5]enkephalin (DPDPE) plus adenosine (200 uM) and lidocaine (500 uM) in 10 mM glucose containing Krebs Henseleit buffer (pH 7.7 at 37° C.). The hearts arrested in 17 to 23 sec and remained arrested throughout the 30 min protocol. Time to first beat after arrest during reperfusion was about 1 minute. After 30 min heart rate was 90-112%, pressures were over 95%, aortic flow was over 80% and coronary flow was over 85% of pre-arrest values. After 45 min heart rate was 86-110%, pressures were over 90%, aortic flow was 80% and coronary flow was 77-90% of pre-arrest values. This example shows that a delta-1-opioid agonist plus a potassium channel opener or adenosine agonist and a local anaesthetic arrests, protects and preserves the heart.

### Example 6

### The Effect of A, L and AL Solution on In Vitro Superoxide Generation by Activated Neutrophils

To isolate neutrophils, peripheral canine blood (200 ml) was mixed with 45 ml of anticoagulating agents, which included 1.6% citric acid and 2.5% sodium citrate (pH 5.4) and 100 ml of 6% dextran solution in buffered Hanks, balanced salt solution (HBSS). PMNs were isolated using the Ficoll-Pacque (Sigma Chemical, St. Louis, Mo.) technique. The cells were adjusted to ˜9×10⁷ cells/ml. Final suspensions contained 94±1% neutrophils, and cell viability averaged 99±0.5% as determined by trypan blue exclusion. Superoxide anion (—O.2) production by neutrophils stimulated by platelet activating factor (100 nmol) were determined by measuring the superoxide dismutase-inhibitable reduction of ferricytochrome c to ferrocytochrome c spectrophotometrically at 550 nm using a V-Max Microtiter Plate Reader (Molecular Devices, Palo Alto, Calif.). The indicated concentrations of lidocaine (L) or adenosine (ADO) were added before neutrophils were stimulated with PAF. Concentrations indicated are final concentrations in each cuvette.

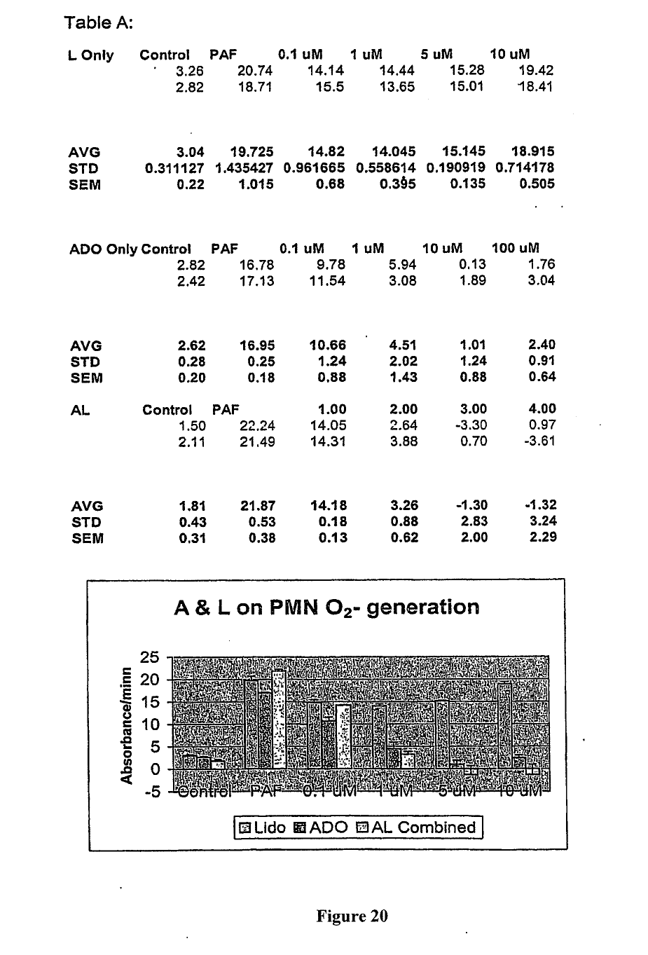

Results and interpretation: The data are shown in the FIG. 20. Absorbance on the Y-axis indicates superoxide production which is maximally stimulated with platelet activating factor (PAF). The concentrations of drugs are on the X-axis. Both L and ADO are in the micromolar range as indicated on the table from which the data are taken. In the combination group, 0.1 uM=0.1 uM L+0.1 uM ADO; 1 uM=1 uM L+1 uM ADO; 5 uM=5 uM L+10 uM ADO; 10 uM=10 uM L+100 uM ADO. The data indicate that A and L individually reduce superoxide anion generation. However, A and L in combination act synergistically to decrease superoxide anion generation at the higher concentration (1 uM, 5 uM and 10 uM) range. AL in combination appear to confer greater protection against superoxide production than A and L alone in activated neutrophils. A lower concentration of each drug canto provide complete inhibition of neutrophil-derived superoxide anions. These results support the proposal that AL may have potent antiinflammatory actions in arrest, protection and preservation of organs, tissues and cells. It is further proposed that adenosine and lidocaine in combination with type IV phosphodiesterase (PDE) inhibitors, or with non-steroidal anti-inflammatory drug or their nitric oxide donors (eg. flurbiprofen or its NO-donating derivative, HCT1026 (2-fluoro-a-methyl[1,1′-biphenyl]-4-acetic acid, 4-(nitrooxy)butyl ester), or AL plus nitric oxide donor (e.g. nitroprusside) may further produce enhanced inhibition of inflammation.

### Example 7

### Effect of Mode of Cardioplegia Delivery (One Shot, Continuous and Intermittent) at Normothermia Using al Cardioplegia in the Isolated Non-Injured Rat Heart, and the Beneficial Effect of Nifedipine Plus AL Using ‘One’ Shot in the Healthy Rat Heart.

Adult Male Sprague-Dawley rats (approx 350 g) were obtained as per the previous examples.

Krebs-Henseleit Perfusion buffer: Modified Krebs Henseleit buffer contained 10 mM glucose; 117 mM NaCl, 5.9 mM KCl, 25 mM NaHCO₃, 1.2 mM NaH₂PO₄, 1.12 mM CaCl₂ (free Ca²⁺=1.07 mM), 0.512 mM MgCl₂ (free Mg²⁺=0.5 mM), pH 7.4 at 37° C. The perfusion buffer was filtered using a one micron (1 uM) membrane and then bubbled vigorously with 95% O₂/5% CO₂ for a pO₂ above 600 mmHg. The perfusion buffer was not recirculated.
Adenosine and lidocaine arrest solution: 200 uM adenosine plus 500 uM lidocaine in 10 mM glucose containing Krebs Henseleit buffer (pH 7.7 at 37° C.). The AL arrest solution was filtered using 0.2 uM filters and maintained at 37° C. The arrest solution was not actively bubbled with 95% O₂/5% CO₂ hence the higher pH (The average pO₂ of the solution was 131 mmHg and pCO₂ of 5-10 mmHg).
Adenosine and lidocaine (AL) arrest solution plus Calcium antagonist nifedipine (RBI N-114. MW 346.34): 52 nM nifedipine plus 200 uM adenosine plus 500 uM lidocaine (AL) in 10 mM glucose containing Krebs Henseleit buffer (pH 7.7 at 37° C.). The AL arrest solution was filtered using 0.2 uM filters and maintained at 37° C. The arrest solution was not actively bubbled with 95% O₂/5% CO₂ hence the higher pH (The average pO₂ of the solution was 131 mmHg and pCO₂ of 5-10 mmHg). 0.0015 g Nifedipine was added to 0.84 ml DMSO (5.156 mM) and 5 ul of this solution was added to 500 ml of AL arrest solution. Final concentration of Nifedipine is 52 nM and DMSO 0.001%. Note: nM=nanomolar
Langendorff and Working Rat Heart preparation: Hearts were rapidly removed from anaesthetised rats and immediately placed in ice-cold Krebs-Henseleit buffer. Excess tissue was removed and the heart was connected via the aorta to a standard Langendorff apparatus with a perfusion pressure of 90 cm H₂O (68 mmHg). After tying off the pulmonary veins and superior and inferior vena cava to minimize leaks (<1 ml/min), the pulmonary artery was cannulated. The preparation was then switched to the working mode and the heart was not placed in a thermostated jacket. The preload was preset at 10 cm H₂O (7.6 mmHg) and the afterload 100 cm H₂O (76 mmHg). Hearts were stabilised for 30 minutes before arrest. Heart rate, aortic pressure, coronary flow, aortic flow and oxygen consumption were measured before, during and following the protocol.

Aortic pressure was measured continuously using a pressure transducer (UFI Instruments, Morro Bay, Calif.) coupled to a MacLab 2e (ADI Instruments). Systolic and diastolic pressures and heart rate were calculated from the pressure trace using the MacLab software. Arterial and venous perfusate pO₂ and pCO₂, pH and ions (Ca²⁺, Cl⁻, and Na⁺) were measured using a Ciba-Corning 865 blood gas machine. Coronary flow and aortic flow were measured in volumetric cylinders. The initial criteria for exclusion of working hearts during the 30 min equilibration period (before arrest) was a heart rate less than 200 beats/min, a systolic pressure less than 100 mmHg and coronary flow less than 10 ml/min. No pacing or cardiac massage was employed during the recovery phase in the working mode.

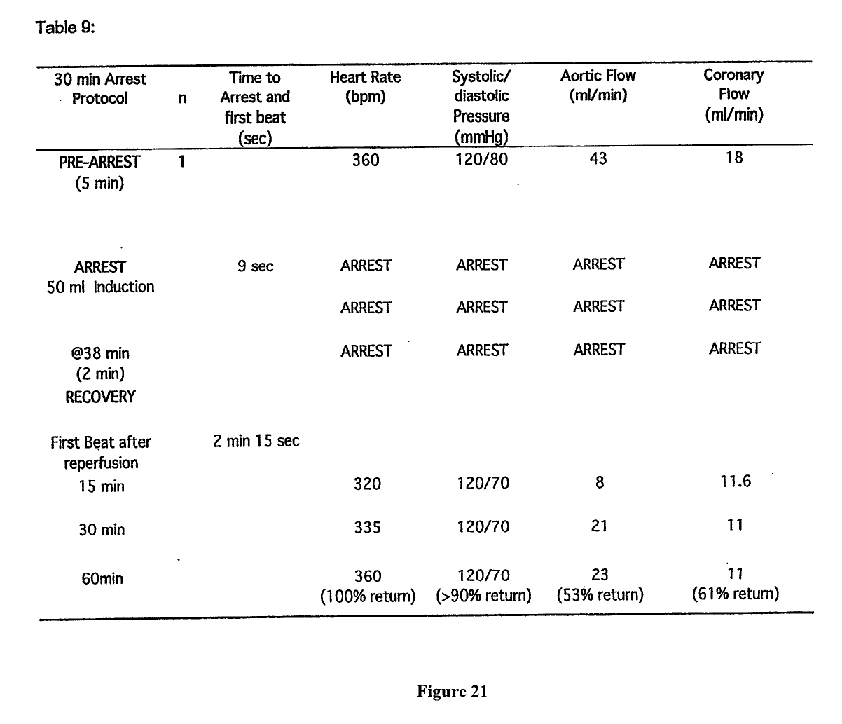

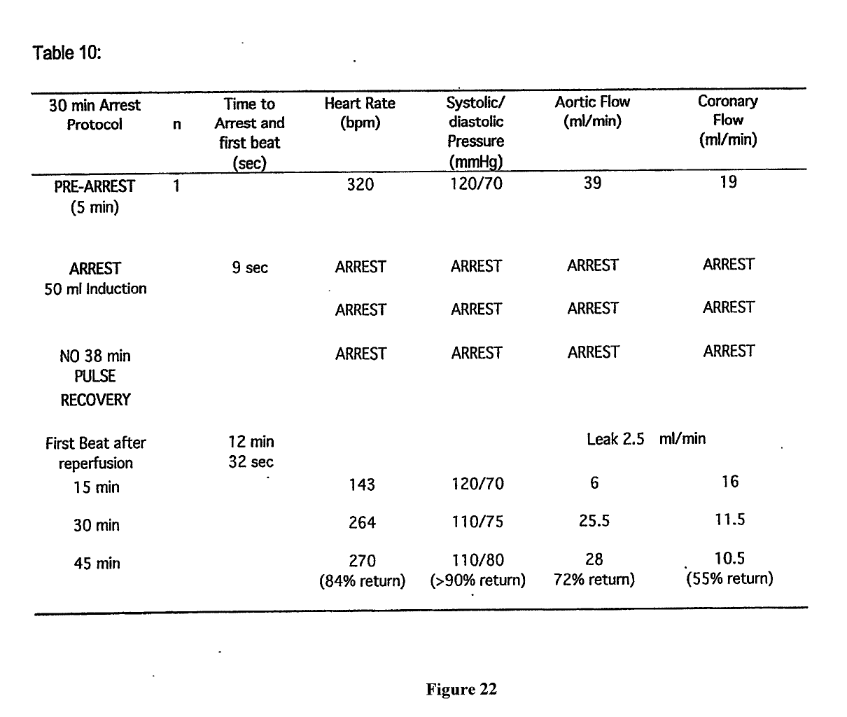

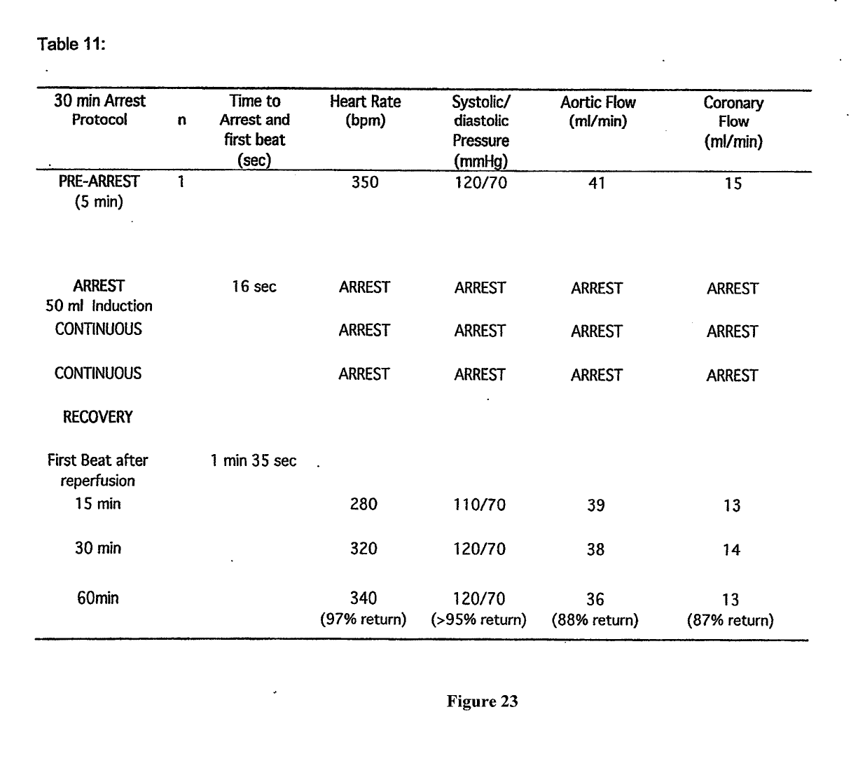

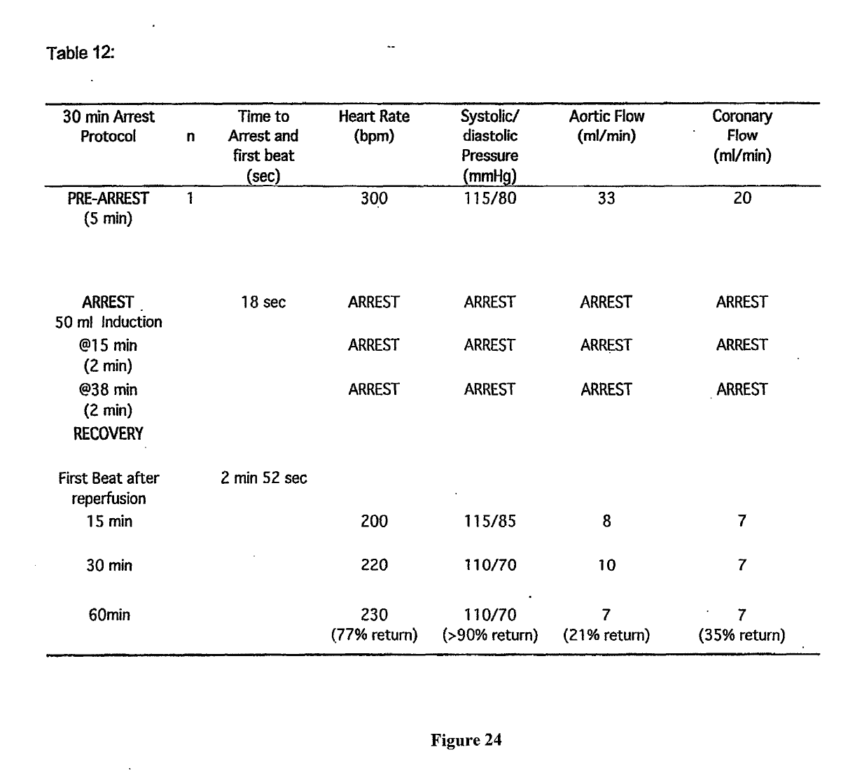

Mode of Cardioplegic delivery and 40 min Arrest Protocol: The hearts were then switched to Langendorff mode and 50 ml of cardioplegia was delivered at 37° C. at a constant pressure head of 90 cm H₂O (68 mmHg).
1(a) ‘One’ shot AL ONLY (NORMOTHERMIA): After 50 ml delivery, the aorta is cross-clamped throughout the arrest period and the clamp released at the end with a further 2 min infusion pulse delivered just prior to reanimation. Another ‘one’ shot strategy is to administer ‘one’ induction at the beginning, clamp the aorta and keep clamped for the entire arrest period and no further terminal 2 min pulse prior to switching into working mode results shown in Table 9 (FIG. 21).
(b) ‘One’ shot AL plus nifedipine (NORMOTHERMIA) results shown in Table 10 (FIG. 22).
(2) Continuous AL delivery ONLY (NORMOTHERMIA): Cardioplegia is delivered continuously for the arrest period results shown in Table 11 (FIG. 23).
(3) Intermittent AL delivery ONLY (NORMOTHERMIA): For the intermittent protocol, 50 ml induction volume is administered and the aorta was cross-clamped for 15 min after which it was released to deliver a 2 min infusion pulse of cardioplegia solution and the clamp reapplied shown in Table 12, (FIG. 24).

Hearts were then returned to working mode and recovery was monitored for 45 to 60 min at 37° C. Protection was assessed by measuring a number of physiological parameters including aortic and coronary flows, heart rate, recovery of systolic and diastolic pressures which were compared to baseline values.

Table 9 (FIG. 21) summarises the results ‘One’ shot AL alone (NORMOTHERMIA) in hearts arrested with 200 uM adenosine and 500 uM lidocaine in 10 mM glucose containing Krebs Henseleit buffer (pH 7.7 at 37° C.). The heart arrested in 9 sec and remained arrested throughout the 40 min protocol. Time to first beat after arrest during reperfusion was 2 min 15 sec. After 15 min heart rate was 89%, pressures were over 95%, aortic flow was 19% and coronary flow was over 64% of pre-arrest values. After 30 min heart rate was 93%, pressures were over 90%, aortic flow was 49% and coronary flow was 61% of pre-arrest values. At 60 min heart rate was 100%, pressures were over 90%, aortic flow was 53% and coronary flow was 61% of pre-arrest values. This example shows that one shot of AL alone at normothermia arrests the heart with 50 to 60% recovery of aortic and coronary flows.

Table 10 (FIG. 22) summarises the results of ‘One’ shot AL plus 50 nM nifedipine in 10 mM glucose containing Krebs Henseleit buffer (pH 7.7 at 37° C.). The heart arrested in 9 sec min and remained arrested throughout the 40 min protocol. Time to first beat after arrest during reperfusion was 12 min and 32 sec. After 15 min heart rate was 45%, pressures were over 95%, aortic flow was 15% and coronary flow was over 84% of pre-arrest values. After 30 min heart rate was 83%, pressures were over 90%, aortic flow was 65% and coronary flow was 84% of pre-arrest values. At 45 min heart rate was 84%, pressures were over 90%, aortic flow was 72% and coronary flow was 55% of pre-arrest values. This example shows that a calcium channel blocker plus AL appears to improve aortic flow recovery at 45 minutes into reperfusion compared to AL alone (Table 1).

Table 11 (FIG. 23) summarises the results of continuous delivery of AL cardioplegia (NORMOTHERMIA). AL cardioplegia comprised 200 uM adenosine and 500 uM lidocaine in 10 mM glucose containing Krebs Henseleit buffer (pH 7.7 at 37° C.). The heart arrested in 16 sec and remained arrested throughout the 40 min protocol. Time to first beat after arrest during reperfusion was 1 min 35 sec. After 15 min heart rate was 89%, pressures were over 95%, aortic flow was 80% and coronary flow was over 95% of pre-arrest values. After 30 min heart rate was 91%, pressures were over 95%, aortic flow was 93% and coronary flow was 93% of pre-arrest values. At 60 min heart rate was 97%, pressures were over 95%, aortic flow was 88% and coronary flow was 87% of pre-arrest values. This example shows that continuous delivery of AL provides excellent arrest, protection and preservation.

Table 12 (FIG. 24) summarises the results of INTERMITTENT delivery of AL cardioplegia (NORMOTHERMIA). AL cardioplegia comprised 200 uM adenosine and 500 uM lidocaine in 10 mM glucose containing Krebs Henseleit buffer (pH 7.7 at 37° C.). The heart arrested in 18 sec and remained arrested throughout the 40 min protocol. Time to first beat after arrest during reperfusion was 2 min 52 sec. After 15 min heart rate was 67%, pressures were over 95%, aortic flow was 24% and coronary flow was over 35% of pre-arrest values. After 30 min heart rate was 73%, pressures were over 95%, aortic flow was 21% and coronary flow was 35% of pre-arrest values. At 60 min heart rate was 77%, pressures were over 90%, aortic flow was 21% and coronary flow was 35% of pre-arrest values. This example shows that intermittent delivery of AL provides arrest, protection and preservation but less functional recovery compared to either one-shot AL, one shot AL plus nifedipine or AL continuous at normothermia temperatures.

### Example 8

### Effect of AL Cardioplegia Containing Different Concentration of Magnesium, Chloride and on Function in the Healthy Rat Heart

Adult Male Sprague-Dawley rats (˜350 g) were obtained as per previous example.

Krebs-Henseleit Perfusion buffer: Modified Krebs Henseleit buffer contained 10 mM glucose; 117 mM NaCl, 5.9 mM KCl, 25 mM NaHCO₃, 1.2 mM NaH₂PO₄, 1.12 mM CaCl₂ (free Ca²⁺=1.07 mM), 0.512 mM MgCl₂ (free Mg²⁺=0.5 mM), pH 7.4 at 37° C. The perfusion buffer was filtered using a one micron (1 uM) membrane and then bubbled vigorously with 95% O₂/5% CO₂ for a pO₂ above 600 mmHg. The perfusion buffer was not recirculated.

Arrest solutions were:

(1) AL cardioplegia containing High Magnesium (16 mM) and high chloride (124.5+32=158 mM) and normal sodium (143 mM): 200 uM adenosine plus 500 uM lidocaine in 10 mM glucose containing otherwise normal Krebs Henseleit buffer (pH 7.7 at 37ᵢC) above but instead of 0.512 mM MgCl2, 16 mM was used. This had the effect also to raise the chloride by 16×2=32 mM
(2) AL cardioplegia containing high Magnesium (16 mM), normal chloride (124.5 mM) and low sodium (111 mM) 200 uM adenosine plus 500 uM lidocaine in 10 mM glucose containing otherwise normal Krebs Henseleit buffer (pH 7.7 at 37ⱼC) above but instead of 0.512 mM MgCl2, 16 mM was used, and 85 mM NaCl was added (not the normal 117 mM NaCl). This had the effect also to lower the sodium by 32 mM to 111 mM.
The AL arrest solutions were filtered using 0.2 uM filters and maintained at 37ᵢC. The arrest solution was not actively bubbled with 95% O₂/5% CO₂ hence the higher pH (The average pO₂ of the solution was 131 mmHg and pCO₂ of 5-10 mmHg).

Langendorff and Working Rat Heart preparation: Hearts were rapidly removed from anaesthetised rats and immediately placed in ice-cold Krebs-Henseleit buffer. Excess tissue was removed and the heart was connected via the aorta to a standard Langendorff apparatus with a perfusion pressure of 90 cm H₂O (68 mmHg). After tying off the pulmonary veins and superior and inferior vena cava to minimize leaks (<1 ml/min), the pulmonary artery was cannulated. The preparation was then switched to the working mode and the heart was not placed in a thermostated jacket. The preload was preset at 10 cm H₂O (7.6 mmHg) and the afterload 100 cm H₂O (76 mmHg). Hearts were stabilised for 30 minutes before arrest. Heart rate, aortic pressure, coronary flow, aortic flow and oxygen consumption were measured before, during and following the protocol.

Aortic pressure was measured continuously using a pressure transducer (UFI Instruments, Morro Bay, Calif.) coupled to a MacLab 2e (ADI Instruments). Systolic and diastolic pressures and heart rate were calculated from the pressure trace using the MacLab software. Arterial and venous perfusate pO₂ and pCO₂, pH and ions (Ca²⁺, Cl⁻, and Na⁺) were measured using a Ciba-Corning 865 blood gas machine. Coronary flow and aortic flow were measured in volumetric cylinders. The initial criteria for exclusion of working hearts during the 30 min equilibration period (before arrest) was a heart rate less than 200 beats/min, a systolic pressure less than 100 mmHg and coronary flow less than 10 ml/min. No pacing or cardiac massage was employed during the recovery phase in the working mode. The hearts were then switched to Langendorff mode and 50 ml of cardioplegia was delivered at 37ᵢC at a constant pressure head of 90 cm H₂O (68 mmHg). The heart temperatured drifted down during arrest to about 22° C. The mode of cardioplegia delivery was intermittent or otherwise known as multidose. 50 ml induction volume is administered and the aorta was cross-clamped for 15 min after which it was released to deliver a 2 min infusion pulse of cardioplegia solution and the clamp reapplied. Another 2 min pulse was administered at 28 min just prior to reperfusion. Hearts were then returned to working mode and recovery was monitored for 45 to 60 min at 37° C. Protection was assessed by measuring a number of physiological parameters including aortic and coronary flows, heart rate, recovery of systolic and diastolic pressures which were compared to baseline values.

Table 13 summarises the results AL cardioplegia containing High Magnesium (16 mM) and high chloride (124.5+32=158 mM) and normal sodium (143 mM). The heart arrested in 13 sec and remained arrested throughout the 30 min protocol. Time to first beat after arrest during reperfusion was 4 min and aortic flow occurred at 12 min. After 15 min heart rate was 78%, pressures were over 95%, aortic flow was 87% and coronary flow was over 83% of pre-arrest values. After 30 min heart rate was 82%, pressures were over 90%, aortic flow was 76% and coronary flow was 77% of pre-arrest values. At 60 min heart rate was 76%, pressures were over 90%, aortic flow was 59% and coronary flow was 70% of pre-arrest values. This example shows that the presence of high magnesium and high chloride in AL during intermittent cardioplegia delivery leads to 59-70% recovery of aortic and coronary flows.

Table 14 summarises the results of AL cardioplegia containing high Magnesium (16 mM), normal chloride (124.5 mM) and low sodium (111 mM). The heart arrested in 8 sec and remained arrested throughout the 30 min protocol. Time to first beat during reperfusion was 12 min and 30 sec. After 15 min heart rate was 80%, pressures were over 95%, aortic flow was 94% and coronary flow was 147% of pre-arrest values. After 30 min heart rate was 86%, pressures were over 95%, aortic flow was 66% and coronary flow was 107% of pre-arrest values. At 45 min heart rate was 90%, pressures were over 95%, aortic flow was 56% and coronary flow was 113% of pre-arrest values. This example shows that AL cardioplegia containing high magnesium (16 mM), normal chloride (124.5 mM) and low sodium (111 mM) appears to have an improvement in return of coronary flow at 15, 30 and 45 min compared to hearts receiving AL plus high magnesium and high chloride and normal sodium. This example implies the high magnesium and low sodium might have a beneficial effect on the coronary vessels.

### Example 9

### Effect of AL on Injured Rat Hearts

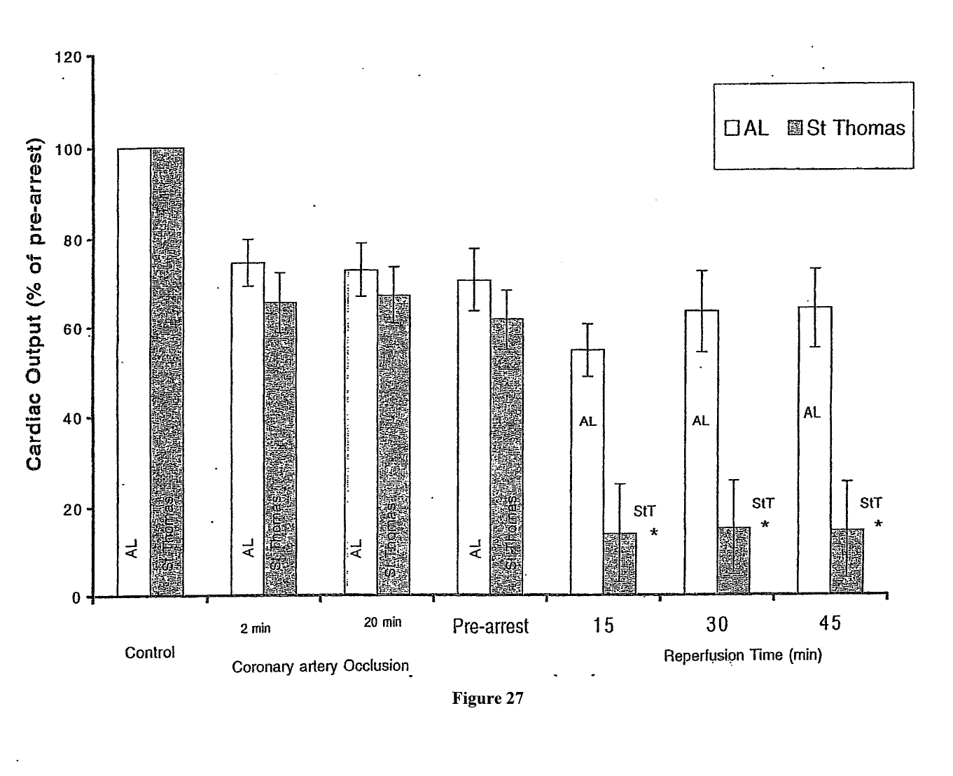

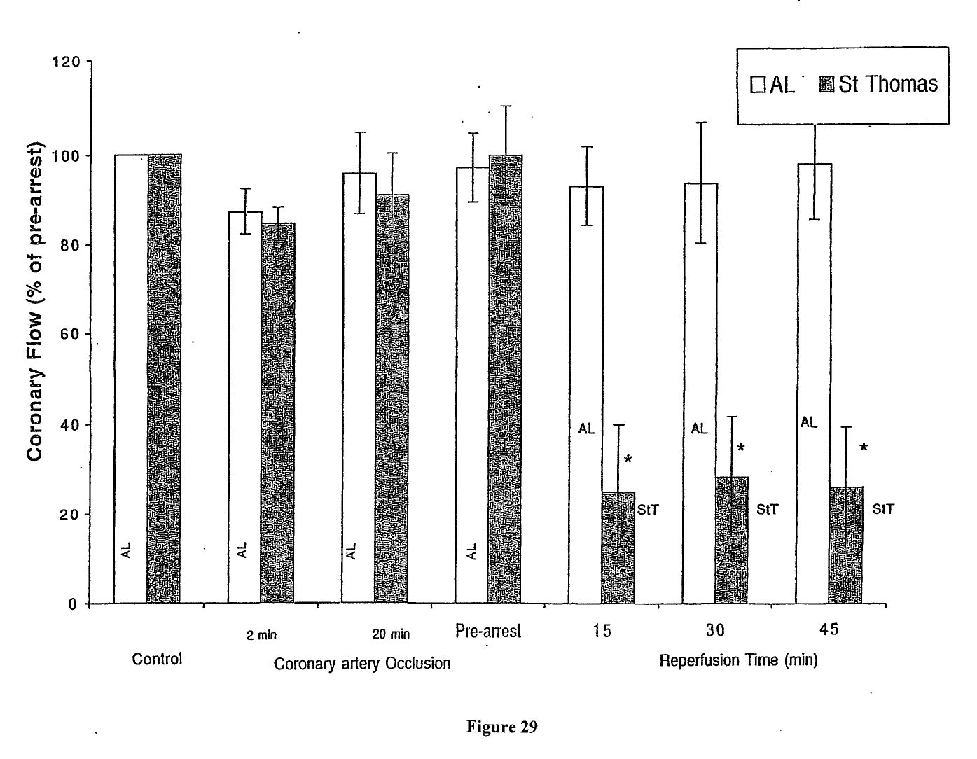

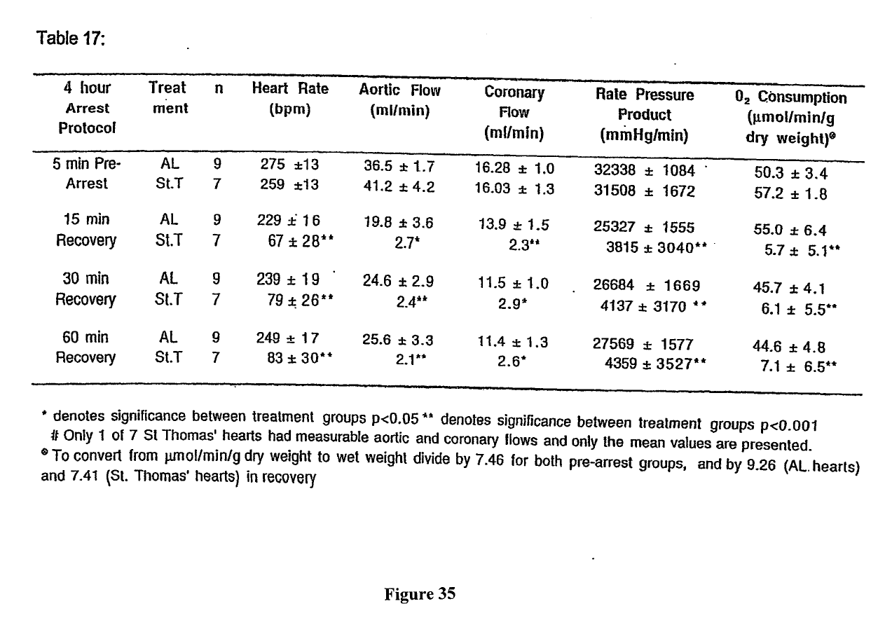

The injured rat hearts and results in FIGS. 27 to 36 were generated as follows. Animals Adult Male Sprague-Dawley rats (˜300 g, n=12) were obtained from Animal Resources Center (Canningvale, Wash.) and JCU's breeding colony. Animals were otherwise prepared as per previous examples

Adenosine and Lidocaine Arrest solution (a composition according to the invention): 200 μM adenosine plus 500 μM lidocaine in 10 mM glucose containing Krebs Henseleit buffer (pH 7.7 at 37° C.). The AL arrest solution was filtered using 0.2 μM filters and maintained at 37° C. The arrest solution was not actively bubbled with 95% O₂/5% CO₂ hence the higher pH (the average pO₂ of the solution was 131 mmHg and pCO₂ of 5-10 mmHg).

Krebs-Henseleit Perfusion buffer: Hearts were perfused in the Langendorff and working mode with a modified Krebs Henseleit buffer containing 10 mM glucose; 117 mM NaCl, 5.9 mM KCl, 25 mM NaHCO₃, 1.2 mM NaH₂PO₄, 1.12 mM CaCl₂ (free Ca²⁺=1.07 mM), 0.512 mM MgCl₂ (free Mg²⁺=0.5 mM), pH 7.4 at 37° C.²⁴. The perfusion buffer was filtered using a one micron (1 μM) membrane and then bubbled vigorously with 95% O₂/5% CO₂ for a pO₂ above 600 mmHg. The perfusion buffer was not recirculated.

Modified St. Thomas' Hospital Solution No 2: NaCl (110 mM), KCl (16 mM), MgCl₂ (16 mM), CaCl₂ (1.2 mM), NaHCO₃ (25 mM) pH 7.8. The buffer was filtered using 0.2 μm filters and maintained at 37° C. The solution was not actively bubbled with 95% O₂/5% CO₂ (The average pO₂ of the solution was 125 mmHg and pCO₂ of 5-10 mmHg). The reason for increasing the bicarbonate concentration from 10 mM to physiological levels of 25 mM was to provide greater buffering capacity¹⁷⁰ thus eliminating the difficulty of adjusting the pH of a weakly buffered solution. The experiments showed no significant differences in heart function after 30 min arrest (n=12) or 4 hours arrest (n=4) between the traditional and modified St. Thomas' Hospital No 2 solution (data not presented). Glucose was not included in St. Thomas solution based on the findings of Hearse and colleagues who showed glucose (with or without insulin) may be deleterious when used as an additive^{151, 171}.

Langendorff and Working Rat Heart preparation: Hearts were rapidly removed from anaesthetised rats and immediately placed in ice-cold Krebs-Henseleit buffer. Excess tissue was removed and the heart was connected via the aorta to a standard Langendorff apparatus with a perfusion pressure of 90 cm H₂O (68 mmHg)¹⁷². After tying off the pulmonary veins and superior and inferior vena cava to minimize leaks (<1 ml/min), the pulmonary artery was cannulated. The preparation was then switched to the working mode and the heart was not placed in a thermostated jacket. The preload was preset at 10 cm H₂O (7.6 mmHg) and the afterload 100 cm H₂O (76 mmHg). Hearts were stabilised for 30 minutes before switching back to Langendorff and administering the arrest solution.

Aortic pressure was measured continuously using a pressure transducer (UFI Instruments, Morro Bay, Calif.) coupled to a MacLab 2e (ADI Instruments). Systolic and diastolic pressures and heart rate were calculated from the pressure trace using the MacLab software. Arterial and venous perfusate pO₂ and pCO₂, pH and ions (Ca²⁺, Cl⁻, and Na⁺) were measured using a Ciba-Corning 865 blood gas machine. Coronary flow and aortic flow were measured in volumetric cylinders. The initial criteria for exclusion of working hearts during the 30 min equilibration period (before ischaemia) was a heart rate less than 200 beats/min, a systolic pressure less than 100 mmHg and coronary flow less than 10 ml/min. No pacing or cardiac massage was employed during the recovery phase in the working mode.

The effect of a composition according to the invention was tested on the isolated working rat heart following 20 min regional ischaemia produced by ligating the left anterior descending (LAD) coronary artery in the working mode at 37° C. Parallel studies have shown that the infarct size after 30 min ligation of the LAD in the rat heart is 60 to 70% of the area of risk. Heart rate, aortic pressure, coronary flow, aortic flow and oxygen consumption were measured at 2 and 20 min during coronary artery occlusion. After 20 min ischaemia, the ligation snare was removed and hearts were reperfused in working mode for 15 min at 37° C. At 15 min, heart rate, aortic pressure, coronary flow, aortic flow and oxygen consumption were measured just before heart arrest. The hearts were then switched to Langendorff mode and 50 ml of one of the tested cardioplegia solutions was delivered at 37° C. at a constant pressure head of 90 cm H₂O (68 mmHg). The aorta was then cross-clamped and the heart remained quiescent for 40 min. At 40 min, the cross-clamp was removed and a further volume of cardioplegia was delivered for 2 min via the aorta. This mode of cardioplegia delivery is a single ‘one shot’ delivery as opposed to ‘intermittent’ (often given as an induction dose plus a 2 min delivery every 20 min throughout the arrest period) or ‘continuous’ delivery which is given throughout the entire arrest period. Hearts were then returned to working mode and recovery was monitored for 45 min at 37° C. Protection was assessed by measuring a number of physiological parameters including aortic and coronary flows, heart rate, recovery of systolic and diastolic pressures which were compared to baseline values. All results are expressed as mean±standard error of the mean (SEM). Statistics were performed separately for each of the 30 min, 2 hour and 4 hour protocols. Two-way ANOVA with repeated measures were used to compare discrete variables (e.g. coronary resistance, aortic flow, systolic and diastolic pressures, oxygen consumption, external work, delivery supply ratio) over multiple time points between the AL and St. Thomas' treatment groups. The alpha level of significance for all experiments was set at P<0.05.

After 30 min equilibration and baseline readings there was no significant difference between the two groups of hearts (in all FIG. 27 to 30). Nor were there functional differences during acute ischaemic injury or during the 15 min reperfusion period prior to arrest between the two groups (in all FIG. 27 to 30). This verifies the uniformity of the acute injury before cardioplegia was administered.

The cardiac output of the heart prior to ischaemic injury, during ischaemic injury (2 and 20 min), at 15 min reperfusion (pre-arrest) and during 45 min recovery following 40 min arrest is shown in FIG. 27. Cardiac output fell by 25-30% as a result of the injury and no significant differences were seen between the two groups. However, following cardioplegia, the AL hearts returned higher cardiac output which after 45 min was not significantly different from the prearrest values. This indicates that little or no left ventricular dysfunction as a result of the AL cardioplegia. In contrast, the St. Thomas' hearts showed markedly reduced function with a return of cardiac output of about 30% of pre-arrest values (or 20% of pre-occlusion or control values).

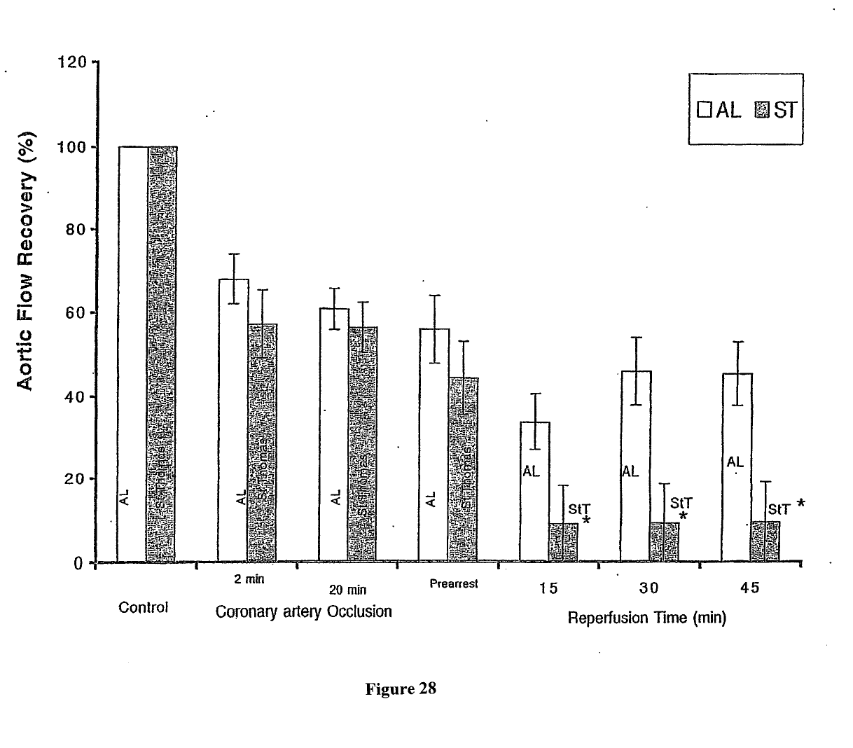

Since cardiac output is the sum of the aortic and coronary flow rates, FIGS. 28 and 3 show that the major factor responsible for the fall in cardiac output in the AL group was a fall in aortic flow, as coronary flow was surprisingly not different from controls. This result suggests that AL provides superior protection against microvascular damage during cardioplegic arrest, and consistent with our prior data showing that AL hearts have little change in vascular resistance during arrest. The data further demonstrates that the injury during ischaemia was probably localised to the left ventricle whose function was compromised because of ligating the left coronary arteries. In contrast, St Thomas' hearts suffered from both microvascular damage (significantly lower coronary flow) and left ventricle myocyte damage (significantly lower aortic flows) compared to AL arrested hearts.

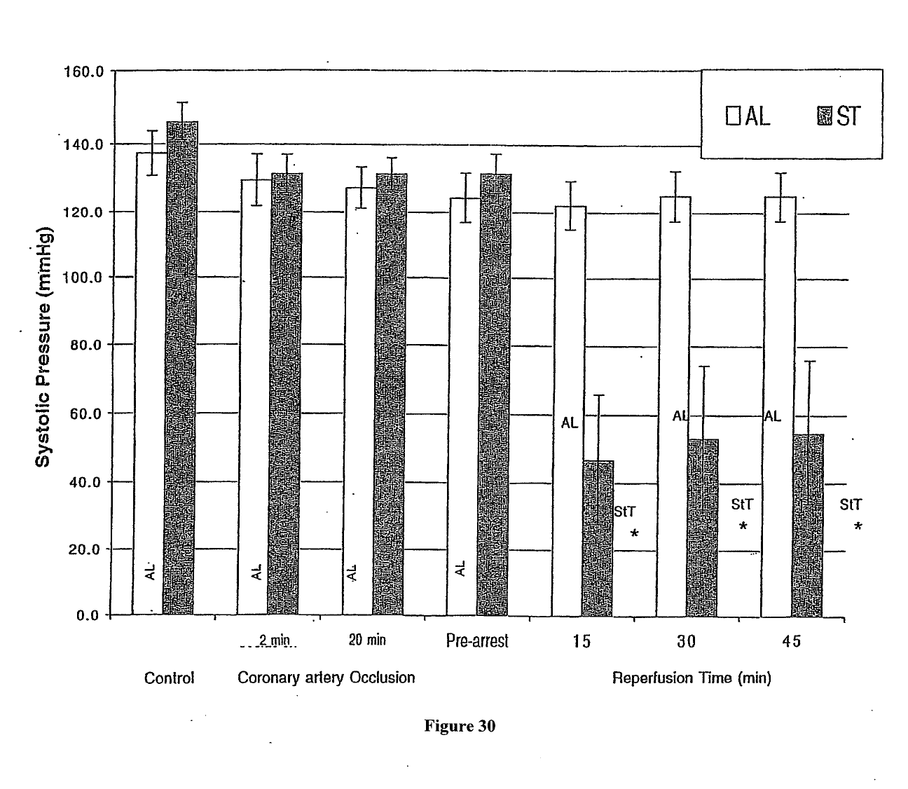

The cardiac output and flow data are also supported by the pressures generated by the heart (FIG. 30). There were no significant differences in systolic pressures in the AL group at any during recovery following arrest but the St. Thomas hearts could only generate 30% of their pre-arrest and pre-injury values. Similar profiles were found for diastolic pressures, heart rate and oxygen consumption and hydraulic work (data not shown).

In summary, the data in FIGS. 27, 28 and 29 show that AL cardioplegia provides superior protection during 40 min ischaemic arrest compared to modified St. Thomas Cardioplegia No 2. While there were no significant differences in cardiac output, aortic and coronary flows before and during regional ischemia at 37° C., the AL hearts recovered with statistically higher function (P<0.05). It is noteworthy that each group of hearts had similar function following ischemia indicating that damage was similar (cardiac output was 60 to 70% of pre-injury values). At 15 min into recovery, the AL hearts recovered about 60% and at 30 min there was 100% recovery relative to pre-arrest values (FIG. 27). St Thomas hearts on the other hand could only generate around 15-20% of pre-arrest cardiac output in recovery. The same differences were seen in systolic pressure from each cardioplegia group (FIG. 30).

Accordingly, it can be seen that the AL composition provides superior arrest, protection and preservation in the acutely injured rat hearts compared to modified St. Thomas hospital solution No 2.

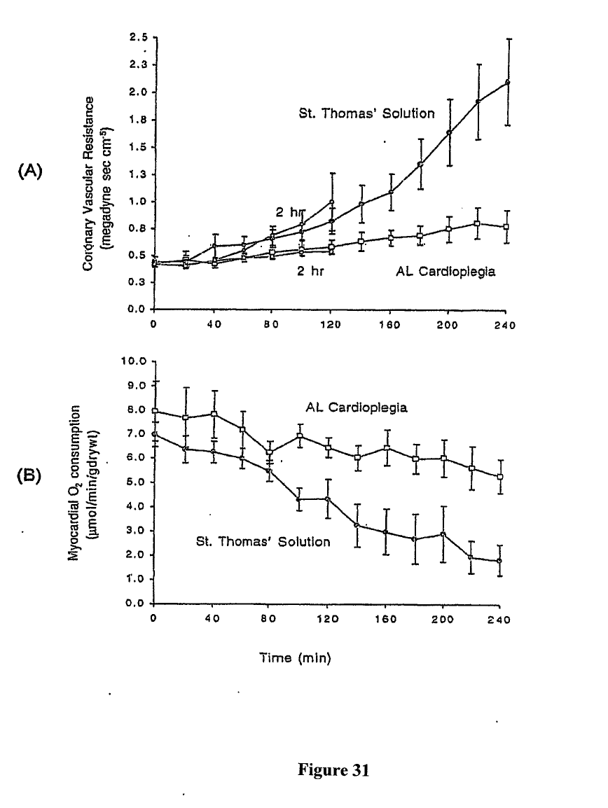

The invention also can be used with healthy hearts as is demonstrated in FIG. 31. FIG. 31 shows data for (a) Coronary Vascular Resistance (CVR) and (b) O₂ consumption during 2 and 4 hr arrest of a healthy heart. CVR was calculated during the 2 min cardioplegia delivery periods. Values are mean±SEM and asterisk shows significance between the two cardioplegia from repeated measures ANOVA (P<0.05). All statistical tests for the 2 and 4 hour AL and St Thomas' arrest protocols were performed separately. For clarity, only the 4 hour arrest data is presented for oxygen consumption and arrest time—no significant differences in the first two hours were found between the 2 and 4 hour arrest protocols.

### Example 9

### Cardioprotective Effects of AL Cardioplegia on Rat Ischaemic Myocardium Compared to St Thomas Solution at 22° to 37° C.

In the following example, the cardioprotective effects of AL cardioplegia on rat ischaemic myocardium are compared to St. Thomas solution at 22 to 37° C. as reflected in FIGS. 31 to 35. Hearts were rapidly removed from anaesthetised rats and immediately placed in ice-cold Krebs-Henseleit buffer. Excess tissue was removed and each heart was connected via the aorta to a standard Langendorff apparatus with a perfusion pressure of 90 cm H₂O (68 mmHg)¹⁶⁹. After tying off the pulmonary veins and superior and inferior vena cava to minimise leaks (<1 ml/min), the pulmonary artery was cannulated. The preparation was then switched to the working mode. The preload was preset at 10 cm H₂O (7.6 mmHg) and the afterload 100 cm H₂O (76 mmHg). Hearts were stabilised for 30 minutes before switching back to Langendorff and administering the arrest solution (see Multidose Cardioplegia delivery below). Heart rate, aortic pressure, coronary flow, aortic flow and oxygen consumption were measured before, during and following arrest.

Animals: Male Sprague-Dawley rats (323±6 g, n=47) were obtained from Animal Resources Center (Canningvale, Wash.) and JCU's breeding colony. Animals were otherwise prepared as per previous examples.

Aortic pressure was measured continuously using a pressure transducer (UFI Instruments, Morro Bay, Calif.) coupled to a MacLab 2e (ADI Instruments). Systolic and diastolic pressures and heart rate were calculated from the pressure trace using the MacLab software. Arterial and venous perfusate pO₂ and pCO₂, pH and ions (Ca²⁺, Cl⁻, and Na⁺) were measured using a Ciba-Corning 865 blood gas machine. Coronary flow and aortic flow were measured in volumetric cylinders. The initial criteria for exclusion of working hearts during the 30 min equilibration period was a heart rate less than 200 beats/min, a systolic pressure less than 100 mmHg and coronary flow less than 10 ml/min. No pacing or cardiac massage was employed during the recovery phase in the working mode. Heart surface temperature was measured using a Cole-Palmer thermistor-thermometer (8402-20) every 30 sec throughout 2 hours of arrest. The thermistor probe was tucked under the left auricle, and placement in the left heart chamber showed similar profiles as sub-auricular placement.

Mode of Multidose Cardioplegic delivery and Experimental Protocol: After the initial induction dose (50 ml) via the aorta in the Langendorff mode at 37° C. and at constant pressure of 70 mmHg, the aorta was cross-clamped directly using a plastic aortic clip. For the 2 and 4 hour arrest protocols, cardioplegia was replenished every 18 min, with replenishment for 2 min, after which the cross-clamp was reapplied. The heart was not contained in a temperature-controlled jacket. This mode of cardioplegia delivery was repeated every 18 min until the heart was switched to the working mode.

Determination of Tissue Water and Haemodynamic Calculations: Total tissue water (%) was determined by the difference in wet weight and dry weight divided by wet weight and multiplied by 100. Powdered tissue from a number of hearts in control, during different times of arrest and following recovery were dried to a constant weight at 85° C. for up to 48 hours as described by Dobson and Cieslar¹⁷³.

Coronary vascular resistance (CVR) in megadyne sec cm⁻⁵ during 2 min cardioplegia delivery was calculated by dividing delivery pressure by flow (volume/sec) using the equation:

$$CVR = \frac{1333 \times mm Hg}{( ml \text{/} sec )} \times {10}^{- 6} \qquad ( 1 )$$

where 1 mmHg=1333 dynes cm⁻² and 10⁻⁶ is a conversion factor from dynes to megadynes

Cardiac oxygen consumption, MVO₂ (μmole O₂/min/g dry wt heart), was calculated from Eqn 2.

$$\begin{matrix}{MVO}_{2 } = \frac{( {p}_{a} {O}_{2} - {p}_{v} {O}_{2 } )}{( Bp - Vp )} \times \frac{α {O}_{2}}{22.40} \times \frac{Coronary Flow}{gmdry wt} \times 1000 \\ = \frac{mmHg}{mmHg} \times \frac{ml \text{/} ml}{ml \text{/} mmol} \times \frac{ml \text{/} min}{gm dry wt} \times 1000\end{matrix} \qquad ( 2 )$$

where pₐO₂ and pᵥO₂ are the partial pressures of oxygen (mmHg) in the arterial and venous perfusion lines. Bₚ is the barometric pressure (760 mmHg) and Vₚ is the water vapour pressure at 37° C.=47.1 mmHg. The molar volume for oxygen at standard temperature and pressure (STP) was 22.40 ml/millimole. αO₂ is the Bunsen solubility coefficient defined as that volume of oxygen gas dissolved in one ml of solution at a specified temperature reduced to STP (0° C., 760 mmHg)²⁹. The αO₂ at 37° C. for human plasma is 0.024 ml/ml¹⁷⁵. Coronary flow is measured in ml/min and heart weight expressed as g dry wt.

$$External cardiac work or power output ( J \text{/} min \text{/} g dry wt heart ) = \frac{( aortic + coronary flow ) ( \times {10}^{- 6} )}{Heart dry weight} \times \frac{average systolic pressure}{1} \times \frac{101 , 325}{760} = \frac{ml \text{/} min \cdot {m}^{3} \text{/} ml}{gm dry wt} \times \frac{mm Hg}{ } \times \frac{{Nm}^{- 2}}{mmHg} \qquad ( 3 )$$

where 10⁶ is required to convert 1 ml into cubic meters and 1 atm=760 mmHg=101,325 Newton meters⁻² (Nm^{31 2}).

All results are expressed as mean±standard error of the mean (SEM). Statistics were performed separately for each of the 30 min, 2 hour and 4 hour protocols. Two-way ANOVA with repeated measures were used to compare discrete variables (e.g. coronary resistance, aortic flow, systolic and diastolic pressures, oxygen consumption, external work, delivery supply ratio) over multiple time points between the AL and St. Thomas' treatment groups. The alpha level of significance for all experiments was set at P<0.05.

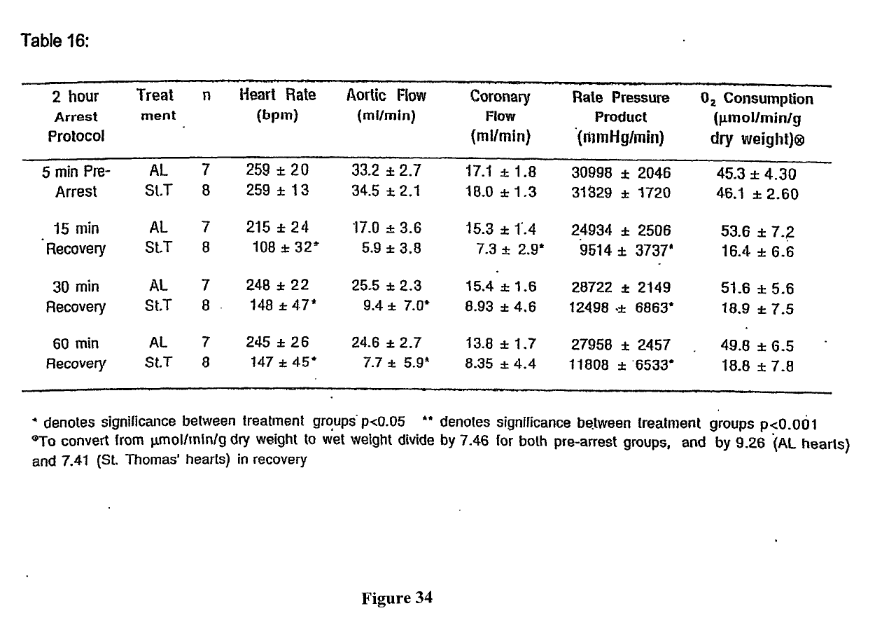

During the pre-arrest (or control period) there was no significant difference in functional parameters between the two groups tested: see Table 16 in FIG. 34 and Table 3 in FIG. 9. Hearts receiving adenosine and lidocaine (AL) cardioplegia achieved electrical and mechanical arrest in 25±2 sec (n=23) compared to 70±5 sec (n=24) for St. Thomas' hearts. After the 50 ml induction volume, 9 out of 23 AL hearts experienced 1.3±0.2 escape beats followed by total arrest. St. Thomas' hearts arrested by becoming progressively weaker (on the basis of developed aortic pressure) over a longer period of time and generally no escape beats were detected.

Functional data from healthy (non-injured) rat hearts arrested using multidose cardioplegia for 2 and 4 hours are also shown in Tables 16 and 17 respectively. St. Thomas' hearts showed significantly lower functional recoveries than hearts arrested with AL cardioplegia. Mean aortic flow was about 22% and 5-10% of pre-arrest values after 2 and 4 hours arrest respectively. Similarly, systolic pressures were 70 and 30 mmHg for 2 and 4 hours respectively. For the 2 hr St. Thomas' group, heart rate, coronary flow, rate-pressure product and O₂ consumption recovered to 40-50% of their pre-arrest values (Table 2). After 60 min of reperfusion, the 4 hour St. Thomas' group had only 32% of heart rate, 23% of systolic pressure, 5% of aortic flow, 16% of coronary flow and 14% of rate-pressure product (Table 3). In direct contrast, the AL Hearts after 2 and 4 hours arrest recovered up to 77% and 70% of their pre-arrest aortic flows respectively, and systolic pressures also reached 113 to 118 mmHg which were 85 to 100% of pre-arrest values, as were oxygen consumption and rate-pressure product (Tables 16 and 17).

Total tissue water content in the pre-arrest working mode was 86.6±1.1% (n=4) and in agreement with earlier studies of Masuda, Dobson and Veech¹⁶⁹. Total tissue water content measured on separate hearts at different times during arrest for St. Thomas' and AL hearts was 87±0.8% (n=8) and 88.7±0.3% (n=14) respectively (P<0.05). There were no significant differences found within each cardioplegia group (ie AL and St Thomas) after 30 min, 2 hr or 4 hr. Separate measurements on different hearts were also made at reperfusion and recovery. The average values during 60 min reperfusion were 86.5±0.6% (n=14) and 89.2±0.3% (n=20) for St. Thomas' and AL hearts respectively. As in arrest, AL hearts had significantly higher post-reperfusion water content than St. Thomas' hearts (P<0.05), but the increased water content had little adverse effect on functional recovery.

In summary, only 50% of St. Thomas hearts (4 out of 8) arrested using multidose cardioplegia for 2 hrs could develop aortic flow against an afterload of 100 cm H₂O, and that percentage dropped to 17% (1 out of 7) in the 4 hour arrest group (FIG. 5). In contrast, 100% of hearts arrested with AL cardioplegia recovered aortic flow against 100 cm H₂O after 2 hours (n=7) and 4 hours (n=9) (Tables 2 and 3).

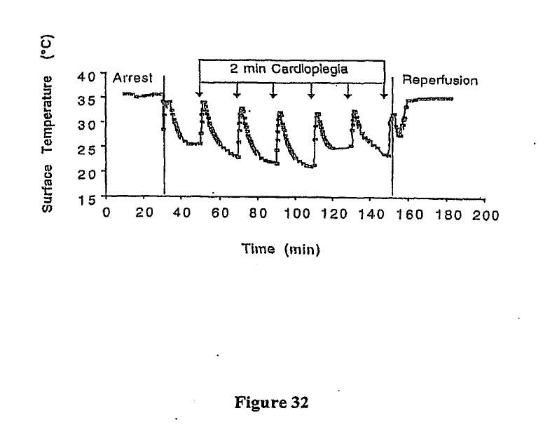

A representative profile of the heart surface temperature for either AL hearts or St. Thomas' hearts is shown in FIG. 32. During the control and 1 hour reperfusion periods, heart temperature was 37° C. but during arrest it cycled between 35 and 22° C. The cycling occurred because the heart was not placed in a temperature-controlled jacket and the peak temperatures correspond to the 2 min delivery of cardioplegia at 37° C. and the valley's to the end of the 18 min ‘on-clamp’ period. The average heart temperature over 2 hours of arrest was 28-30° C. and was not different between AL and St. Thomas' hearts (FIG. 32).

Cardioplegia Delivery Volumes, Coronary Vascular Resistance, and O₂ Consumption during 2 min Off-Clamp: The total cardioplegia volume delivered over 4 hours to AL hearts was 273 ml and 201 ml for St. Thomas' hearts, with the greatest difference between 2 and 4 hours of arrest. For example, at 240 min, 17 ml of cardioplegia was delivered to AL hearts and 7.3 ml to St. Thomas' hearts. Coronary vascular resistance (CVR) at different cardioplegia delivery times during 2 and 4 hour arrest is shown in FIG. 5a. After 2 hours, AL hearts had significantly lower resistance than St. Thomas' Hearts (P<0.05) which helps explain the higher cardioplegia volumes. Decreased CVR is in accord with adenosine's potent coronary vasodilatory properties¹⁶³.

Oxygen consumption was significantly higher during infusions of cardioplegia in AL hearts than the St. Thomas' hearts (FIG. 31b). The higher O₂ consumption (1.5 to 3 times) was due to both an increase in perfusate inflow-outflow (A-V) pO₂ difference (the average A-V pO₂ difference over 4 hours was 83±1.6 mmHg for AL hearts, and 62±1.9 mmHg for St. Thomas' hearts) and higher flows in AL hearts (lower resistance). During infusions of cardioplegia, oxygen consumption in AL and St Thomas' hearts fell to 10% and 5% of their pre-arrest controls respectively.

This example shows that the arresting combination of 200 μM adenosine and 500 μM lidocaine (AL) in normokalemic Krebs-Henseleit at pH 7.4 and 37° C. is superior to hyperkalemic St. Thomas' Hospital solution during prolonged arrest. Rat hearts arrested with multidose AL cardioplegia showed significantly faster electromechanical arrest times (25 vs 70 sec, P<0.05), had lower coronary vascular resistance during cardioplegia infusions (FIG. 31) and superior functional recoveries following arrest.

Without being bound by any theory or mode of action, it is believed that possible reasons for AL's superiority over modified St. Thomas' hospital solution may include: Faster arrest times in AL hearts may lead to better preservation of high-energy phosphates and glycogen, and the maintenance of a high cytosolic phosphorylation ([ATP]/[ADP] [Pᵢ]) ratio and ΔG'_{ATP} and low redox (lactate/pyruvate) ratios.

Second, superior protection may be linked to adenosine's ability to open sarcolemmal ATP-sensitive K⁺ channels of conduction cells and myocytes, shorten the action potential duration, arrest the heart^{161,162} and protect the myocardium during ischaemia^{152,154}. Adenosine's negative chronotropic and dromotropic effects are believed mediated in part by activation of A1 receptors and opening of sarcolemmal ATP-sensitive K⁺ channels (via reduction of adenyl cyclase activity)¹⁶³. This leads to direct and indirect slowing of the heart by inhibiting the pacemaking current in the SA node and slowing atrioventricular (AV) nodal electrical conduction. The A1 receptors are also implicated in the nucleoside's ability to blunt the stimulatory effects of catecholamines, and inhibition of norepinephrine release from nerve terminals¹⁶³. In addition to adenosine's arresting properties, there is substantial experimental evidence for its cardioprotective effects during ischemia such as reductions in infarct size, reduced myocardial stunning, free radical scavenging, anti-inflammatory properties (see below) and improved maintenance of cell metabolism^{163,167}. Activation of ATP-sensitive potassium channels by adenosine is believed to reduce sodium and calcium loading by myocardial cells, and thereby reduce the extent of necrosis, myocardial stunning and reperfusion injury^{160,166,176}. A role for an adenosine-linked opening of mitochondrial ATP-sensitive channels in negative chronotropy and cardioprotection remains to be clarified.

A third reason for AL cardioplegia's superiority is associated with lidocaine's pharmacological action to close Na⁺ fast channels leading to anaesthesia and augmentation of adenosine's arresting effects¹⁵¹. Lidocaine will ‘clamp’ the membrane potential near or at its resting state and, since fewer channels or pumps are activated at polarised potentials, its actions may have energy sparing effects and further reduce Na⁺ and Ca²⁺ loading (see above)^{154,164,166}. The possibility also exists that lidocaine in combination with adenosine may exert additional arresting and cardioprotective actions through some unknown membrane receptor-ligand and/or channel mediation mechanism(s).

A fourth factor contributing to the superior arrest, protection and preservation of AL cardioplegia is adenosine's potent coronary vasodilatory properties leading to reduced coronary vascular resistance and greater delivery of cardioplegia. The lower coronary resistance in AL hearts was not due to reduced tissue oedema (88.7%), nor was St. Thomas's higher resistance and poor performance due to increased oedema (87%). It is particularly noteworthy that total tissue water in crystalloid perfused rat hearts range from 85 to 88%¹⁶⁹, and significantly higher than in situ rat hearts (79%)¹⁷³. Crystalloid perfused hearts undergo a major redistribution of tissue water with the extracellular space over two times the in situ value^{169,173}. Further studies are required to investigate the effect of AL cardioplegia on the regulation of coronary blood flow over prolonged arrest periods and the distribution of water in the interstitial, extracellular and intracellular compartments.

A fifth important factor for AL's superiority is adenosine's¹⁶³ and lidocaine's¹⁶⁸ anti-inflammatory effects which may inhibit cytokine and complement generation that would have a direct effect on myocytes in crystalloid perfused system¹⁶³. The use of adenosine in cell-free systems has been shown to be cardioprotective independent of its effects on neutrophils and other blood-borne inflammatory components¹⁶³. However, adenosine's and lidocaine's anti-inflammatory effects is expected to be of greater importance in blood cardioplegia in intact animal models undergoing cardiopulmonary bypass.

Lastly, AL's superiority over modified St. Thomas' solution may be associated with other compositional differences. AL cardioplegia contains non-depolarising ‘physiological’ potassium concentration similar to the concentration found in blood. High ‘depolarising’ potassium cardioplegia has been linked to metabolic imbalances and dearrangements in sarcolemma ion gradients (particularly Ca²⁺) and left ventricular dysfunction, which is more pronounced at higher arrest temperatures^{152,154-156,177}. In 1991 Yacoub and colleagues also reported that high potassium in St. Thomas' solution or Bretschneider solution resulted in endothelial damage and concentration dependent¹⁷⁸ AL cardioplegia also has a lower more ‘physiological’ magnesium concentration (˜0.5 mM), and while 16 mM in St. Thomas' solution has been shown to be cardioprotective¹⁵¹, the lower concentration did not appear to compromise AL heart's performance. Notwithstanding the complexity of these compositional differences, superior protection and preservation of AL cardioplegia may be due to the presence of exogenous glucose (10 mM). As discussed earlier, glucose was omitted from the St. Thomas solution because Hearse and colleagues showed that its presence was detrimental to recovery^{151,171}, and because commercially available Plegisol (Abbott) does not contain glucose.

The ideal temperature for cardioplegia remains controversial. During open-heart surgery, the surface temperature of the heart under normothermic arrest can drift from 37° C. to 32° C. In an attempt to approximate this in the isolated rat heart model, cardioplegia was delivered at 37° C. for 2 min every 18 min and the heart temperature permitted to drift during the ‘on’ clamp period (intermittent ischaemic period). The heart surface temperature between infusions was 37° C. to 22-24° C. Although hearts receiving both AL and modified St Thomas' cardioplegia experienced the same moderate temperature falls during arrest, the protocol used in the examples is different from current normothermic surgical arrest practices. In this study, shifting from lower arrest temperatures to normothermia at reperfusion may have influenced the recovery of St Thomas hearts. A degree of hyperkalemic-induced heart block cannot be ruled out, but this is considered unlikely as there was no sign of electrical disturbance in the St Thomas' group after 30 min arrest (time to first beat was 2 min 13 sec for St. Thomas' hearts and 2 min 27 sec for AL hearts, and both groups developed aortic flow ˜5 min). In the working heart model, unlike the intact animal, the perfusion pressure is independent of the development of forward flow (or stroke volume), hence, perfusion pressure during the early moments of reperfusion, when contractile effort was unstable and inconsistent, was similar between both groups. Reasons for poor performance in St Thomas' hearts is more likely related to the precipitous rise in coronary vascular resistance during 2 and 4 hours of arrest (up to 4 fold higher than AL hearts) and ischaemia-reperfusion injury. Furthermore, the lower myocardial temperatures achieved between infusions of cardioplegia may have adversely effected the actions of adenosine by blunting the receptor-mediated effects by disengaging the transduction mechanisms¹⁸¹. However, temperature-related uncoupling of receptor transduction mechanisms may occur at more profound levels of hypothermia. In the present study, AL cardioplegia was associated with greater functional recovery despite the moderate temperature decreases between infusions of cardioplegia.

### Example 10

### Effect of Normokalemic AL Cardioplegia on the Membrane Potential in the Heart

In a further example, the effect of normokalemic AL cardioplegia on the membrane potential in the heart is described. This example shows the effect of AL cardioplegia on the membrane potential of healthy (non-injured, non-ischaemic) rat hearts, compared with St Thomas Hospital solution No 2, and 16 mM KCl.

Animals Adult Male Sprague-Dawley rats (˜300 g, n=18) were obtained from Animal Resources Center (Canningvale, Wash.) and JCU's breeding colony. Animals were otherwise prepared as per previous examples.

Estimation of the Myocardial Cell Membrane Potential: Control (non-injured, non-ischaemic, pre-arrest) hearts were freeze-clamped at liquid nitrogen temperatures in the working mode (n=6). A separate group (n=6) was perfused in the working mode and then switched to the Langendorff mode and arrested using St. Thomas' hospital solution No 2 at 37° C. A third separate group (n=6) was perfused in the working mode and then switched to the Langendorff mode and arrested using AL cardioplegia. A few minutes after the hearts were arrested, the hearts were freeze-clamped at liquid nitrogen temperatures and the left ventricular tissue was ground at liquid nitrogen temperatures in a pre-cooled mortar. The tissue was then transferred to liquid nitrogen cooled tubes and kept at −80° C. until use.

Tissue (100 mg) was acid-digested for total potassium measurement and left overnight using the methods described in Masuda, Dobson and Veech¹⁶⁹. The total tissue potassium concentration and intracellular concentration ([K+]in) was measured and calculated using the methods described in Masuda, Dobson and Veech¹⁶⁹. The membrane potential was calculated from the Nernst equation, where Em (membrane potential)=Ek=RT/ZF*log([K+]out/[K+]in), R is the universal gas constant, T is the temperature in Kelvin, Z is the valence of the ion (1+ for potassium) and F is the Faradays constant. The extracellular potassium ([K+]out) is assumed to be the same as in the Krebs-Henseleit (5.9 mM) or cardioplegia (St Thomas, 16 mM; and AL arrest solution, 5.9 mM),

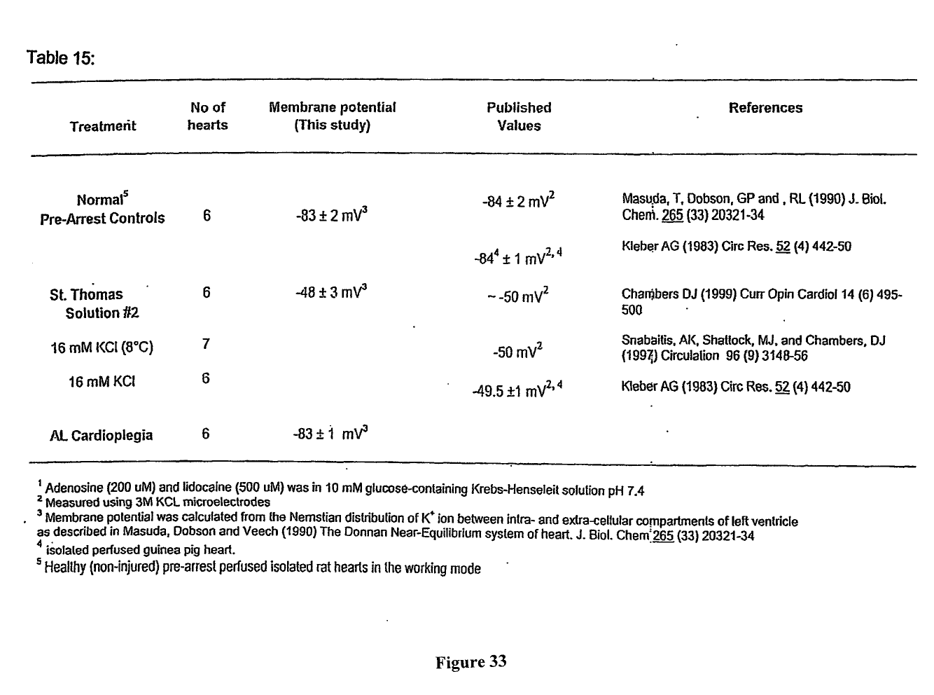

The results show that using the Nernstian distribution of potassium across the heart cell membrane the membrane potential for St Thomas Hospital solution No 2 was −48±3 mV (n=6) (Table 15 in FIG. 33). This result is consistent with the accepted published values based on direct potassium electrode measurements. The published values for hyperkalemic 16 mM K+ solutions such as St Thomas Hospital solution No 2 or potassium chloride (KCl) are −50 and 49.5 mV respectively (Table 15). Using thd Nerstian method, the membrane potential calculated for the non-injured, non-ischaemic, pre-arrested rat heart was −83 mV, which again is consistent with published values for the isolated perfused rat heart or guinea pig heart. Using the Nerstian distribution of potassium, the membrane potential calculated for isolated rat hearts arrested using AL cardioplegia, was −83 mV. The membrane potential for AL arrested hearts is not different from the resting membrane potential. The results also add further support that the Nernst equation and electrodes agree as a measure of the voltage (potential) difference across the myocardial membrane in the control and arrested state.

Thus, it can be seen that one embodiment of the present invention utilising the arresting combination of a K+ channel opener and local anaesthetic (for example, adenosine and lidocaine cardioplegia) does not depolarise the heart cell as high potassium solutions such as St Thomas Hospital solution No 2 or 16 mM KCl (−49.5 to −50 mV), but polarises or ‘clamps’ it close to the resting membrane potential (−83 mV).

Those skilled in the art will appreciate that the invention described above is susceptible to variations and modifications other than those specifically described. It is to be understood that the invention includes all such variations and modifications. The invention also includes all of the steps, features, compositions and products referred to or indicated in this specification, individually or collectively, and any and all combinations of two or of said steps or features.

### REFERENCES

1. Association AH. Heart Stroke Statistical Update. Dallas, Tex. 2002.
2. Bechtel J F M, Christiansen J F, Sievers H H, Bartels C. Low-energy cardioconversion versus medical treatment for the termination of atrial fibrillation fter CABG. Ann Thorac Surg. 2003; 75:1185-1188.
3. Flack J E, Cook J R, May S J, Lemeshow S, Engelman R M, Rousou J A, Deaton D W. Does cardioplegia type affect outcome and survival in patients with advanced left ventricular dysfunction.? Results from CABG Patch Trial. Circulation. 2000; 102:84-89.
4. Buckberg G D. Myocardial protection: an overview. Semin. Thorac. Cardiovasc. Surg. 1993; 5:98-106.
5. Das S N, Chauhan S, Saxena N. Myocardial preservation during cardiac surgery. Ann. Cardiac Anaesthesia. 2002; 5:25-32.
6. McCully J D. Oxygenated multidose delivery of crystalloid esmolol cardioplegia as an alternative to high potassium cardioplegia. J Thorac Cardiovasc Surg. 2002; 124:219-220.
7. Chambers D J, and Hearse, D. J. Cardioplegia and Surgical Ischaemia. In: Sperelakis N, Kurachi, Y., Terzic, A., and Cohen, M. V., ed. Heart Physiology and Pathophysiology. San Diego: Academic Press; 2001:887-926.
8. Mankad P S, Chester, A. H., and Yacoub, M. H. Role of potassium concentration in cardioplegic solutions in mediating endothelial damage. Ann. Thorac. Surg. 1991; 51:89-93.
9. Cohen N M, Damiano, R. J., and Wechsler, A. S. Is there an alternative to potassium arrest? Ann. Thorac. Surg. 1995; 60:858-863.
10. Damiano R J. The electrophysiology of ischaemia and cardioplegia: implication for myocardial protection. J. Card. Surg. 1995; 10:101-109.
11. Lopez J R, Jahangir, R., Jahangir, A., Shen, W. K., and Terzic, A. Potassium channel openers prevent potassium-induced calcium loading of cardiac cells: possible implications in cardioplegia. J. Thorac. Cardiovasc. Surg. 1996; 112:820-831.
12. Cohen N M, Wise, R. M., Wechsler, A. S., and Damiano, R. J. Elective cardiac arrest with a hyperpolarising adenosine triphosphate-sensitive potassium channel opener: A novel form of myocardial protection? J. Thorac. Cardiovasc. Surg. 1993; 106:317-328.
13. Reimer K A, Lowe, J. E., Rasmussen, M. M., and Jennings, R. B. The wavefront phenomenon of ischaemic cell death, I: myocardial infarct size vs. duration of coronary occlusion in dogs. Circulation. 1977; 56:786-794.
14. Jennings R B, and Reimer, K. A. Lethal Myocardial Ischaemic Injury. Amer. J. Pathol. 1981; 102:241-255.
15. Braunwald E, Kloner R A. The stunned myocardium: prolonged, postischaemic ventricular dysfunction. Circulation. 1982;66:1146-1149.
16. Zhao Z O, and Vinten-Johansen, J. Myocardial apoptosis and ischaemic preconditioning. Cardiovasc Res. 2002; 55:438-455.
17. Bolli R, and Marban, E. Molecular and cellular mechanisms of myocardial stunning. Physiol Rev. 1999; 79:609-634.
18. Bolli R. The late phase of preconditioning. Circ Res. 2000; 87:972-983.
19. Zhao Z Q, Nakamura, M, Wang, N. P, Velez, D. A., Hewan-Lowe, K. O., Guyton, R. A., and Vinten-Johansen, J. Dynamic progression of contractile and endothelial dysfunction and infarct extension in the late phase of reperfusion. J Surg Res. 2000; 94:133-144.
20. Frangogiannis N G, Smith., C. W., and Entman, M. L. The inflammatory response in myocardial infarction. Cardiovasc Res. 2002; 53:31-47.
21. Lasley R D, Mentzer R M. Dose-dependent effects of adenosine on interstitial fluid adenosine and postischaemic function in the isolated rat heart. J. Pharmacol Exp. Ther. 1998; 286:806-811.
22. Vinten-Johansen J, Thourani, V. H., Ronson, R. S. Jordan, J. E., Zhao, Z. Q., Nakamura, M., Velez, D., and Guyton, R. A. Broad-spectrum cardioprotection with adenosine. Ann. Thorac. Surg. 1999; 68:1942-1948.
23. McCully J D, Uematsu M, Parker R A, Levitsky S. Adenosine-enhanced ischaemic preconditioning provides enhanced postischaemic recovery and limitation of infarct size in the rabbit heart. J. of Thorac. Cardiovasc. Surg. 1998; 116:154-162.
24. Avkiran M. Protection of the ischaemic myocardium by Na+/H+ exchange inhibitors: potential mechanisms of action. Basic Res Cardiol. 2001; 96:306-311.
25. Mubagwa K, and Willem, F. Adenosine, adenosine receptors and myocardial protection: An updated overview. Cardiovasc. Res. 2001; 52:25-39.
26. Kloner R A, and Jennings, R. B. Consequences of brief ischemia: stunning, preconditioning, and their clinical implications: part 1. Circulation. 2001; 104:2981-2989.
27. Baxter G F, and Ferdinandy, P. Delayed preconditioning of myocardium: current perspectives. Basic Res CardioL 2001; 96:329-344.
28. Vinten-Johansen J, Thourani V H. Myocardial protection: an overview. J Extra Corpor Technol. 2000; 32:38-48.
29. Homeister J W, Hoff, P. T., Fletcher, D. D., and Lucchesi, B. R. Combined adenosine and lidocaine administration limits myocardial reperfusion injury. Circulation. 1990; 82:595-608.
30. Vander Heide R S, Reimer K A. Effect of adenosine therapy at reperfusion on myocardial infarct size in dogs. Cardiovascular Research. 1996; 31:711-718.
31. Garratt K N, Holmes D R, Molina-Viamonte V, Reeder G S, Hodge D O, Bailey K R, Lobl J K, Laudon D A, Gibbons R J. Intravenous adenosine and lidocaine in patients with acute myocardial infarction. American Heart Journal. 1998; 136:196-204.
32. Mahaffey K W, Puma, J. A., Barbagelata, N. A., DiCarli, M. F., Leesar, M. A., Browne, K. F., Eisenberg, P. R., Bolli, R., Casas, A. C., Molina-Viamonte, V., Orlandi, C., Blevins, R., Gibbins, R. J., Califf, R. M., and Granger, C. B. Adenosine as an adjunct to Thrombolytic Therapy for acute myocardial infarction. J. Amer. College Cardiol. 1999; 34:1711-1720.
33. Dobson G P, and Jones, M. W. Adenosine and Lignocaine: a new concept in non-depolarising surgical arrest, protection and preservation. J. Thoracic Cardiovas Surgery (accepted for publication). 2003.
34. Dobson G P, and Jones, M. W. Adenosine and Lignocaine: a new concept in cardiac arrest and preservation. Ann Thorac Surg. 2003;75: S746.
35. Ely S W, and Berne, R. M. Protective effects of adenosine in myocardial ischaemia. Circulation. 1992; 85:893-904.
36. Baxter G F. Role of adenosine in delayed preconditioning of myocardium. Cardiovasc Res. 2002; 55:483-494.
37. De Jonge R, De Jong J W, Keijzer E, Bradamante S. The role of adenosine in preconditioning. Pharmacol Ther. 2000; 87:141-149.
38. Linden J. Molecular approach to adenosine receptors: receptor-mediated mechanisms of tissue protection. Ann Rev Pharmacol. Toxicol. 2001; 41:775-787.
39. De Jonge R, Out, M., Maas, W. J., De Jong, J. W. Preconditioning of rat hearts by adenosine A1 or A3 receptor activation. Eur J. Pharmacol. 2002; 441:165-172.
40. Martynyuk A E, Seubert C N, Zima A, Morey T E, Belardinelli L, Lin G, Cucchiara R F, Dennis D M. Contribution of I(K,ADO) to the negative dromotropic effect of adenosine. Basic Res Cardiol. 2002; 97:286-294.
41. Murry C E, Jennings R B, Reimer K A. Preconditioning with ischemia: a delay of lethal cell injury in ischaemic myocardium. Circulation. 1986; 74:1124-1136.
42. Yellon D M, and Downey, J. M. Spotlight on preconditioning. Cardiovasc Res. 2002; 55:425-428.
43. Przyklenk K, Kloner R A. Ischaemic preconditioning: exploring the paradox. Progress in Cardiovascular Diseases. 1998; 40:517-547.
44. Yellon D M, Alkhulaifi A M, Pugsley W B. Preconditioning the human myocardium. Lancet. 1993;342:276-277.
45. Vaage J, and Valen, G. Preconditioning and cardiac surgery. Ann Thorac Surg. 2003;75:S709-714.
46. Kuzuya T, Hoshida S, Yamashita N, Fuji H, Oe H, Hori M, Kamada T, Tada M. Delayed effects of sublethal ischemia on the acquisition of tolerance to ischemia. Circ Res. 1993; 72:1293-1299.
47. Przyklenk K, Darling C E, Dickson E W, Whittaker P. Cardioprotection ‘Outside the Box’: the evolving paradigm of remote preconditioning. Basic Res Cardiol. 2003; 98:1-9.
48. Yellon D M, and Dana, A. The preconditioning phenomenon: a tool fo the scientist or clinical reality. Circ. Res. 2000; 87:543-550.
49. Cohen M V, Basines, C. P., and Downey, J. M. Ischaemic Preconditioning: from adenosine to K_{ATP} channel. Ann. Rev. Physiol. 2000; 62:79-109.
50. Kloner R A, and Jennings, R. B. Consequences of brief ischemia: stunning, preconditioning, and their clinical implications: part 2. Circulation. 2001; 104:3158-3167.
51. Post H, and Heusch, G. Ischaemic Preconditioning: Experimental facts and clinical perspective. Minerva Cardioangiol. 2002; 50:569-605.
52. Lasley R D, Zhou, Z., Hegge, J. O., Bunger, R., and Mentzer, R. M. Jr. Adenosine attenuates in vivo myocardial stunning with minimal effects on cardiac energetics. Basic Res Cardiol. 1998; 93:303-312.
53. Auchampach J A, Rizvi, A., Qiu, Y., Tang, X. L., Maldonado, C., Teschner, S., Bolli, R. Selective activation of A3 adenosine receptors with N6-(3-iodobenzyl)adenosine-5′-N-methyluronamide protects against myocardial stunning and infarction without hemodynamic changes in conscious rabbits. Circ Res. 1997; 80:800-809.
54. Toyoda Y, Friehs, I., Parker, R. A., Levitsky, S., McCully, J. D. Differential role of sarcolemmal and mitochondrial K(ATP) channels in adenosine-enhanced ischaemic preconditioning. Am J Physiol (Heart Circ Physiol.). 2000; 279:H2694-2703.
55. Saltman A E, Aksehirli T O, Valiunas V, Gaudette G R, Matsuyama N, Brink P, Krukenhamp I B. Gap junction uncoupling protects the heart against ischemia. J. Thorac. Cardiovas. Surg. 2002; 124:371-376.
56. Bell R M, Smith C C, Yellon D M. Nitric oxide as a mediator of delayed pharmacological (A(1) receptor triggered) preconditioning; is eNOS masquerading as iNOS? Cardiovasc Res. 2002; 53:405-413,
57. Hudspeth D A, Nakaniski, K., Vinen-Johansen, J., and others. Adenosine in blood cardioplegia prevents postischaemic dysfunction in ischaemically injured hearts. Ann. Thorac. Surg. 1994;58:1637-1644.
58. De Jong J W, Van der Meer, P., Van Loon, H., Owen, P., Opie, L. H. Adenosine as an adjunct to potassium cardioplegia: Effect of function, energy metabolism, and electrophysiology. J. Thorac. Cardiovasc. Surg. 1990; 100:445-454.
59. Jovanovic A, Alekseev, A. E., Lopez, J. R., Shen, W. K., and Terzic, A. Adenosine prevents hyperkalemia-induced calcium loading in cardiac cells: relevance to cardioplegia. Ann. Thorac. Surg. 1997; 63:153-161.
60. Thourani V H, Ronson, R. S., VanWylen, D. G. Myocardial protection with adenosine given at reperfusion is superior to adenosine-enhanced cardioplegia. Circulation. 1998; 98:13217-3218.
61. Cohen G, Feder-Elituv, R., Lazetta, J., Bunting, P., Mallidi, H., Bozinovski, J., Deemer, C., Christakis, G. T., Cohen, E. A., Wong, B. I., McLean, R. D., Myers, M., Morgan, C. D., Mazer, C. D., Smith, T. S., Goldman, B. S., Naylor, C. D., Fremes, S. E. Phase 2 studies of adenosine cardioplegia. Circulation. 1998; 98:II225-233.
62. Li G-R, Ferrier G R. Effects of lidocaine on reperfusion arrhythmias and electrophysiological properties in an isolated ventricular muscle model of ischemia and reperfusion. J. Pharmacol. Exp. Ther. 1991; 257:997-1004.
63. Tosaki A, Balint S, Szekeres L. Protective effect of lidocaine against ischemia and reperfusion-induced arrhythmias and shifts of myocardial sodium, potassium and calcium content. Journal of Cardiovascular Pharmacology. 1988; 12:621-628.
64. Zamponi G W, Doyle, D. D., and French, R. J. Fast Lidocaine block of cardiac and skeletal muscle sodium channels: one site with two routes of access. Biophys. J. 1993; 65:80-90.
65. Opie L H. Drugs for the heart. 4th ed. London, New York: W.B. Saunders; 1995.
66. Das K C, Misra H P. Lidocaine: a hydroxyl radical scavenger and singlet oxygen quencher. Mol. Cell. Biochem. 1992; 115:179-185.
67. Tanaka Y, Kamibayashi M, Yamashita Y, Imai T, Tanaka H, Nakahara T, Ishii K, Shigenobu K. Evidence for the possible involvement of Ca2+ entry blockade in the relaxation by class I antiarrhythmic drugs in the isolated pig coronary-smooth muscle. Naunyn Schmiedebergs Arch Pharmacol. 2002; 365:56-66.
68. Hollmann M W, Wieczorek, K. S., Berger, A., and Durieux, M. E. Local anesthetic inhibition of G protein-coupled receptor signaling by interference with Galpha(q) protein function. Mol. Pharmacol. 2001; 59:294-301.
69. Hollmann M W, Difazio, C. A., and Durieux, M. E. Ca-signaling G-protein-coupled receptors: a new site of local anesthetic action? Reg Anesth Pain Med. 2001; 26:565-571.
70. Stewart G J. Neutrophils and deep vein venous thombosis. Haemostasis. 1993; 23:127-140.
71. Tobias M D, Henry C, Augostides Y G. Lidocaine and bupivavaine exert differential effects on whole blood coagulation. J. Clin Anaesth. 1999;11:52-55.
72. Fredholm B B, Ijzerman AP, K. A. J, Klotz K N, Linden J. International union of Pharmacology. XXV. Nomenclature and Classification of Adenosine receptors. PharmocoL Rev. 2001; 53:527-552.
73. Dhalla A K, Shryock J C, Shreeniwas R, Belardinelli L. Pharmacology and therapeutic applications of A(1) adenosine receptor ligands. Curr Top Med. Chem. 2003; 3:369-385.
74. Lerman B B, Ellenbogen K A, Kadish A, Platia E, Stein K M, Markowitz S M, Mittal S, Slotwiner D, Scheiner M, Iwai S, Belardinelli L, M. J, Shreeniwas R, Wolff A A. Electrophysiologic effects of a novel selective adenosine A1 agonist (CVT-510) on atrioventricular nodal conduction in humans. J Cardiovasc Pharmacol Ther. 2001; 6:237-245.
75. Hayes E S. Adenosine receptors and cardiovascular disease: the adenosine-1 receptor (A1) and A1 selective ligands. Cardiovascular Toxicology. 2003; 3:1-18.
76. Fraser H, Gao Z, Ozeck M J, Belardinelli L. N-[3-(R)-tetrahydrofuranyl]-6-aminopurine riboside, an A1 adenosine receptor agonist, antagonizes catecholamine-induced lipolysis without cardiovascular effects in awake rats. J Pharmacol Exp Ther. 2003; 305:225-231.
77. Beukers M W WM, Von Frijtag Drabbe Kunzel J K, Klaasse E C, Ijzerman A P, Koomen G J. N6-cyclopentyl-2-(3-phenylaminocarbonyltriazene-1-yl)adenosine (TCPA), a very selective agonist with high affinity for the human adenosine A1 receptor. J Med. Chem. 2003; 46:1492-1503.
78. Baraldi P G, Romagnoli R, Pavani M G, Nunez Mdel C, Tabrizi M A, Shryock J C, Leung E, Moorman A R, Uluoglu C, lannotta V, Merighi S, PA. B. Synthesis and biological effects of novel 2-amino-3-naphthoylthiophenes as allosteric enhancers of the A1 adenosine receptor. J Med. Chem. 2003; 46:794-809.
79. Lorenzen A, Beukers M W, van der Graaf P H, Lang H, van Muijlwijk-Koezen J, de Groote M, Menge W, Schwabe U, IJzerman A P. Modulation of agonist responses at the A(1) adenosine receptor by an irreversible antagonist, receptor-G protein uncoupling and by the G protein activation state. Biochem Pharmacol. 2002; 64:1251-1265.
80. Peart J N, Gross G J. Adenosine and Opioid Receptor-Mediated Cardioprotection in the Rat: Evidence for Cross-Talk Between Receptors. Am J Physiol Heart Circ Physiol. 2003; In Press.
81. Ozcan C, Bienengraeber, M., Dzeja, P. P., and Terzic A. Potassium channel openers protect cardiac mitochondria by attenuating oxidant stress at reoxygenation. Am J Physiol Heart Circ Physiol. 2002; 282:H531-539.
82. Barrett T D, Hayes E S, Yong S L, Zolotoy A B, Abraham S, Walker M J A. Ischaemia selectivity confers efficacy for suppression of ischaemia-induced arrhythmias in rats. Eur. J. Pharmacol. 2000; 398:365-374.
83. Bauer A, Becker R, Voss F, Senges J C, Kraft P, Schreiner K D, Keubler W, Schoels W. Effects of acute ischemia, early extrabeats and propafenone on complex activation patterns in intact and ischaemic canine hearts. Life Sciences. 2003; 72:2751-2767.
84. Opitz C F, Mitchell G F, Pfeffer M A, Pfeffer J M. Arrhythmias and death after coronary artery occlusion in the rat. Circulation. 1995; 92:253-261.
85. Walker M J A, Curtis M J, Hearse D J, Campbell R W F, Janse M J, Yellon D M, Cobbe S M, Coker S J, Harness J B, Harron D W G, Higgins A J, Julian D G, Lab M J, Manning A S, Northover B J, Parratt J R, Riemersma R A, Riva E, Russell D C, Sheridan D J, Winslow E, Woodward B. The Lambeth Convention: guidelines for the study of arrhythmias in ischaemia, infarction, and reperfusion. Cardiovascular Research. 1988; 22:447-455.
86. Fishbein M C, Meerbaum S, Rit J, Lando U, Kanmatsuse K, Mercier J C, Corday E, Ganz W. Early phase acute myocardial infarct size quantification: validation of the triphenyltetrazolium chloride tissue enzyme staining technique. American Heart Journal. 1981; 101:593-600.
87. Schreieck J, and Richardt, G. Endogenous adenosine reduces the occurrence of ischemia-induced ventricular fibrillation in rat heart. J. Mol. Cell. Cardiol. 1999; 31:123-134.
88. Gorge B, Kurz, T., Katus, H. A., and Richardt, G. Endogenous adenosine suppresses norepinephrine-induced ventricular arrhythmias in rat heart. Basic Res. Cardiol. 1998; 93:264-268.
89. Lasley R D, and Mentzer, R. M. Protective effects of adenosine in the reversibly injured heart. Ann of Thorac Surg. 1995; 60:843-846.
90. Toombs C P, McGeee, S., Johnston, W. E. Myocardial protective effects of adenosine:infarct size reduction with pretreatment and continued receptor stimulation during ischaemia. Circulation. 1992; 86:986-994.
91. Thornton J D, Liu, G. S., Olsson, R. A., and Downey, J. M. Intravenous pretreatment with A1-selective adenosine analogues protects the heart against infarction. Circulation. 1992; 85:659-665.
92. Du X J, Dart, A. M., and Reimer, R. A. Sympathetic activation and increased extracellular potassium: synergistic effects on cardiac potassium uptake and arrhythmias. J. Cardiovasc. Pharmacol. 1993; 21:977-982.
93. Lee H T, LaFaro, R. J., and Reed, G. E. Pretreatment of human myocardium with adenosine during open heart surgery. J Card Surg. 1995; 10:665-676.
94. Lagerkranser M, Bergstrand G, Gordon E, lrestedt L, Lindquist C, Stange K, Sollevi A. Cerebral blood flow and metabolism during adenosine-induced hypotension in patients undergoing cerebral aneurism surgery. Acta Anaesthesiol. Scand. 1989; 33:15-20.
95. Ravingerova T, Slezak, J., Tribulova, N., Dzurba, A., Uhrik, B., and Ziegelhoffer, A. Free oxygen radicals contribute to high incidence of reperfusion-induced arrhythmias in isolated rat heart. Life Sci. 1999; 65:1927-1930.
96. Cronstein B N, Levin R I, Philips M, Hirschhorn R, Abramson S B, Weissmann G. Neutrophil adherence to endothelium is enhanced via adenosine A1 receptors and inhibited via adenosine A2 receptors. J. Immunol. 1992; 148:2201-2206.
97. Hyvonen P M, Kowolik M J. Dose-dependent suppression of the neutrophil respiratory burst by lidocaine. Acta Anaesthesiol. Scand. 1998; 42:565-569.
98. Brooks W W, Conrad, C. H., and Morgan, J. P. Reperfusion induced arrhythmias following ischemia in intact rat heart: role of intracellular calcium. Cardiovasc. Res. 1995; 29:536-542.
99. Curtis M J, Pugsley, M. K., and Walker, M. J. Endogenous chemical mediators of ventricular arrhythmias in ischaemic heart disease. Cardiovasc. Res. 1993; 27:703-719.
100. Lu H R, Yang, P., Remeysen, P., Saels, A., Dai, D. Z., and De Clerck, F. Ischaemialreperfusion-induced arrhythmias in anaesthetized rats: a role of Na+ and Ca2+ influx. Eur. J. Pharmacol. 1999; 365:233-239.
101. Eng S, Maddaford, T. G., Kardami, E., and Pierce, G. N. Protection against myocardial ischaemic/reperfusion injury by inhibitors of two separate pathways of Na+ entry. J. Mol. Cell. Cardiol. 1998; 30:829-835.
102. Lerman B B. Response of nonreentrant catecholamine-mediated ventricular tachycardia to endogenous adenosine and acetylcholine. Evidence for myocardial receptor-mediated effects. Circulation. 1993; 87:382-390.
103. Yoshida T, Engelman R M, Engelman D T, Rousou J A, Maulik N, Sato M, Elliott G T, Das D K. Preconditioning of the swine heart with monophosphoryl lipid A improves myocardial preservation. Ann. Thorac. Surg. 2000; 70:895-900.
104. Jordan J E, Thourani, V. H., Auchampach, J. A., Robinson, J. A., Wang, N.-P., and Vinten-Johansen, J. A3 adenosine receptor activation attenuates neutrophil function and neutrophil-mediated reperfusion injury. Am. J. Physiol. 1999; 277:H1895-H1905.
105. Pitarys C J, Virmani, R., Vildibill, H. D., Jackson. E. K., and Forman, M. B. Reduction of myocardial reperfusion injury by intravenous adenosine administered during the early reprefusion period. Circulation. 1991; 83:237-247.
106. Babbit D, Virmani, R., and Formann, M. B. Intracoronary adenosine administered after reperfusion limits vascular injury after prolonged ischemia in the canine model. Circulation. 1989; 80:1388-1399.
107. Zhao Z Q, Nakamura, M, Wang, N. P, Wilcox, J. N., Shearer, S., Guyton, R. A., and Vinten-Johansen, J. Administration of adenosine during reperfusion reduces injury of vascular endothelium and death of myocytes. Coron. artery dis. 1999; 10:617-628.
108. Nakamura M, Wang, N. P., Zhao, Z. Q., Wilcox, J. N., Thourani, V., Guyton, R. A., and Vinten-Johansen, J. Preconditioning decreases Bax expression, PMN accumulation and apoptosis in reperfused rat heart. Cardiovasc. Res. 2000; 45:661-670.
109. Hollmann M W, Gross, A., Jelacin, N., and Durieux, M. E. Local anaesthetic effects on priming and activation of human neutrophils. Anesthesiology. 2001; 95:113-122.
110. Vitola J V, Forman, M. B., Holsinger, J. P., Atkinson, J. B., and Murray, J. J. Reduction of myocardial infarct size in rabbits and inhibition of activation of rabbit and human neutrophils by lidocaine. Am. Heart J. 1997; 133:315-322.
111. Golino P, Maroko, P. R., and Carew, T. E. Efficacy of platelet depletion in counteracting the detrimental effect of acute hypercholesterolemia on infarct size and the no-reflow phenomenon in rabbits undergoing coronary artery occlusion-reperfusion. Circulation. 1987; 76:173-180.
112. Olafsson B, Forman M B, Puett D W. Reduction of reperfusion injury in the canine preparation by intracoronary adenosine. Circulation. 1987; 76:1135-1145.
113. Engler R L, Schmid-Schonbein G W, Pavelec R S. Leukocyte capillary plugging in myocardial ischemia and reperfusion in the dog. Am. J. Pathol. 1983; 111:98-111.
114. Dobson G P, and Himmelreich, U. Heart Design: free [ADP] scales with absolute mitochondrial and myofibrillar volumes from mouse to human. Biochim Biophys Acta (Bioenergetics). 2002;1553:72-78.
115. Opitz C F, Finn, P. V., Pfeffer, M. A., Mitchell, G. F., and Pfeffer, J. M. Effects of reperfusion on arrhythmias and death after coronary artery occlusion in the rat: increased electrical stability independent of myocardial salvage. J Am Coil CardioL 1998; 32:261-267.
116. Schaper W, Bernotat-Danielowski, S., Nienaber, C., and Shaper, J. Collateral circulation. In: Fozzard H A, Jennings, R. B., haber, E., Katz, A. M., and Morgan, H. E., ed. The Heart and Cardiovascular System. New York: Raven Press; 1986:1427-1460.
117. Barth E, Stammler G, Speiser B, Schaper J. Ultrastructural quantitation of mitochondria and myofilaments in cardiac muscle from 10 different animal species including man. J. Mol. Cell. Cardiol. 1992; 24:669-681.
118. Yao Z, Gross G J. A comparison of adenosine-induced cardioprotection and ischaemic preconditioning in dogs: efficacy, time course, and role of K_{ATP} channels. Circulation. 1994;89:1229-1236.
119. Wilson R F, Wyche K, Christensen B V, Zimmer S, Laxson D D. Effects of adenosine on human coronary arterial circulation. Circulation. 1990; 82:1595-1606.
120. Joshi S, Duong H, Mangla S, Wang M, Libow A D, Popilskis S J, Ostapkovich N D, Wang T S, Young W L, Pile-Spellman J. In nonhuman primates intracarotid adenosine, but not sodium nitroprusside, increases cerebral blood flow. Anesth. Analg. 2002; 94:393-399.
121. Powell J R, Foster J, Patterson J H, Cross R, Wargin W. Effect of duration of lidocaine infusion and route of cimetidine administration on lidocaine pharmacokinetics. Clin. Pharm. 1986; 5:993-998.
122. Cardinal R, Janse M J, van Eeden I, Werner G, d'Alnoncourt C N, Durrer D. The effects of lidocaine on intracellular and extracellular potentials, activation, and ventricular arrhythmias during acute regional ischemia in the isolated porcine heart. Circ. Res. 1981; 49:792-806.
123. Balser J R. The cardiac sodium channel: gating function and molecular pharmacology. J. Mol. Cell. cardiol 2001; 33:599-613.
124. Delmas P, Coste B. Na⁺ channel Na_{V}1.9: in search of a gating mechanism, Trends in Neurosciences. 2003; 26:55-57.
125. Ovize M, Aupetit J F, Riofol G, Loufoua J, Andre-Fouet X, Minaire Y, Faucon G. Ischaemic preconditoning reduces infarct size but accelerates time to ventricular fibrillation in ischaemic pig heart. Amer. J. Physiol. 1995; 269:H72-H79.
126. Lerman B B, Belardinelli, L. Cardiac electrophysiology of adenosine. Basic and clinical concepts. Circulation. 1991; 83:1499-1509.
127. Pelleg A, Kutalek S P. Adenosine in the mammalian heart: nothing to get excited about. Trends Pharmacol. Sci. 1997; 18:236-238.
128. Light P E, Kanji H D, Manning Fox J E, French R J. Distinct myoprotective roles of cardiac sarcolemmal and mitochondrial KATP channels during metabolic inhibition and recovery. FASEB. J. 2001; 15:2586-2594.
129. Liu G S, Thornton J, Van Winkle D M, Stanley A W H, Olsson R A, Downey J M. Protection against infarction afforded by preconditioning is mediated by A₁ adenosine receptors in rabbit heart. Circulation. 1991; 84:350-356.
130. Auchampach J A, and Gross, G. Adenosine A1 receptors, KATP channels. and ischaemic preconditioning on dogs. Amer. J. Physiol. 1993; 264 (Heart Circ Physiol):H1327-H1336.
131. Carr C S, Hill R J, Masamune H, Kennedy S P, Knight D R, Tracey W R, Yellon D M. Evidence for a role for both the adenosine A1 and A3 receptors in the protection of isolated human atrial muscle against simulated ischaemia. Cardiovas. Res. 1997; 36:52-59.
132. Gross G J, and Fryer, R. M. Sarcolemmal versus mitochondrial ATP-sensitive K⁺ channels and myocardial protection. Circ. Res. 1999; 84:973-979.
133. Sato T, Sasaki, N., O'Rourke, B., Marban, E. Adenosine primes the opening of mitochondrial ATP-sensitive potassium channels: a key step in ischaemic preconditioning? Circulation. 2000; 102:800-805.
134. O'Rourke B. Mitochondrial K_{ATP} channels in preconditioning. Circ. Res. 2000; 87:845-855.
135. Krieg T, Qin Q, McIntosh E C, Cohen M V, Downey J M. ACh and adenosine activate PI3-kinase in rabbit hearts through transactivation of receptor tyrosine kinases. Am J. Physiol. 2002; 283:H2322-2330.
136. Mubagwa K. Does adenosine protect the heart by acting on the sarcoplasmic reticulum. Cardiovas. Res. 2002; 53:286-289.
137. De Mendonca A, Sebastiao A M, Ribeiro J A. Adenosine: does it have a neuroprotective role after all? Brain Research Rev. 2000; 33:258-274.
138. Nasser F N, Walls, J. T., Edwards, W. D., and Harrison, C. E. Lidocaine-induced reduction in size of experimental myocardial infarction. Am. J. Cardiol. 1980; 46:967-975.
139. Lesnefsky E J, VanBenthuysen K M, McMurtry I F, Shikes R H, Johnston R B J, Horwitz L D. Lidocaine reduces the canine infarct size and decreases release of a lipid peroxidation product. Journal of Cardiovascular Pharmacology. 1989; 13:895-901.
140. Kojima M, Miura M. Protective effect of lidocaine on the ischaemic-reperfused rat heart: a phosphorus 31 nuclear magnetic resonance study. Basic Research in Cardiology. 1991; 86:179-187.
141. Kambouris N G, Nuss, H. B., Johns, D. C., Marban, E., Tomaselli, G. F., and Balser, J. R. A revised view of cardiac sodium channel “blockade” in the long-QT syndrome. J Clin Invest. 2000; 105:1133-1140.
142. Barrett T D, Hayes E S, Walker M J A. Lack of sensitivity for ventricular and ischaemic tissue limits the antiarrhythmic actions of lidocaine, quinidine and felcamide against ischaemia-induced arrhythmias. Eur. J. Pharmacol. 1995; 285:229-238.
143. Avkiran M. Adenosine A(1) receptor stimulation inhibits alpha(1)-adrenergic activation of the cardiac sarcolemmal Na+/H+ exchanger. Br. J. Pharmacol. 2000; 131:659-662.
144. Zhang Y H, Hinde A K, Hancox J C. Anti-adrenergic effect of adenosine on Na+/Ca2+ exchange current recorded from guinea-pig ventricular myocytes. Cell Calcium. 2001; 29:347-358.
145. Casati C, Forlani A, Lozza G, Monopoli A. Hemodynamic changes do not mediate the cardioprotection induced by the A1 adenosine receptor agonist CCPA in the rabbit. Pharmacol Res. 1997; 35:51-55.
146. Ringer S. A further contribution regarding the influence of the different constituents of the blood on the contraction of the heart. J. Physiol. 1883; 4:29-42.
147. Hooker D R. On the recovery of the heart in electric shock. Amer. J. Physiol. 1929;91:305-328.
148. Melrose D G, Dreyer, B., Bentall, H. H., and Baker, J. B. E. Elective cardiac arrest. Lancet. 1955; 2:21-22.
149. Bretschneider H J, Huubner, G. and Knoll, D. Myocardial resistance and tolerance to ischemia: physiological and biochemical basis. J. Cardiovas. Surg. 1975; 16:241-260.
150. Buckberg G D. Update of Current Techniques of myocardial protection. Ann. Thorac. Surg. 1995; 60:805-814.
151. Chambers D J, and Hearse, D. J. Cardioplegia and Surgical Ischaemia. In: Sperelakis N, Kurachi, Y., Terzic, A., and Cohen, M. V., ed. Heart Physiology and Pathophysiology. San Diego: Academic Press; 2001:887-926.
152. Cohen N M, Damiano, R. J., and Wechsler, A. S. Is there an alternative to potassium arrest? Ann. Thorac. Surg. 1995; 60:858-863.
153. Jayawant A M, Stephenson, E. R., Matte, G. S., Prophet, G. A., LaNoue, K. F., Griffiths, J. W., and Damiano, R. J. Potassium channel opener cardioplegia is superior to St. Thomas' solution in the intack animal. Ann. Thorac. Surg. 1999; 68:67-74.
154. Lopez J R, Jahangir, R., Jahangir, A., Shen, W. K., and Terzic, A. Potassium channel openers prevent potassium-induced calcium loading of cardiac cells: possible implications in cardioplegia. J. Thorac. Cardiovasc. Surg. 1996;112:820-831.
155. Jayawant A M, and Damiano, R. J. The superiority of pinacidil over adenosine cardioplegia in blood perfused isolated hearts. Ann. Thorac. Surg. 1998; 66.
156. Suleiman M S, Halestrap, A. P., and Griffiths, E. J. Mitochondria: a target for myocardial protection. Pharmacol. Ther. 2001; 89:29-46.
157. Menasche P, Kevelaitis, C., Grousset, C., Piwnica, A., and Bloch, G. Preconditioning with potassium channel openers. A new concept for enhancing cardioplegic protection. J. Thorac. Cardiovasc. Surg. 1995; 110:1606-1613.
158. Gross G J, and Auchampach, J. A. Role of ATP dependent potassium channels in myocardial ischaemia. Cardiovasc. Res. 1992; 26:1011-1016.
159. Ducko C T, Stephenson, E. R., Jayawant, A. M., Vigilance, D. W., and Damiano, R. J. Potassium channel openers: are they effective as pretreatment or additives to cardioplegia? Ann. Thorac. Surg. 2000; 69:1363-1368.
160. Hu K, Li, G-R., and Nattel, S. Adenosine-induced activation of ATP-sensitive K⁺ channels in excised membrane patches is mediated by PKC. Amer. J. Physiol. 1999; 276:H488-H495.
161. Foker J E, Einziz, S., and Wang, T. Adenosine metabolism and myocardial preservation. J. Thoras. Cardiovasc. Surg. 1980; 80:506-516.
162. Schubert R, Vetter, H., Owen, P., Reichart, B., and Opie, L. H. Adenosine Cardioplegia. J. Thorac. Cardiovasc. Surg. 1989; 98:1057-1065.
163. Vinten-Johansen J, Thourani, V. H., Ronson, R. S. Jordan, J. E., Zhao, Z. Q., Nakamura, M., Velez, D., and Guyton, R. A. Broad-spectrum cardioprotection with adenosine. Ann. Thorac. Surg. 1999; 68:1942-1948.
164. Haworth R A, Goknur, A. B., Berkoff, H. A. Inhibition of Na—Ca exchange by general anaesthetics. Circ. Res. 1989; 65:1021-1028.
165. Hearse D J, O'Brian, K., and Braimbridge, M. V. Protection of the myocardium during ischaemic arrest: dose respose curves for procaine and lignocaine solutions. J. Thorac. Cardiovasc. Surg. 1981; 81:873-879.
166. Jovanovic A, Lopez, J. R., Alekseev, A. E., Shen, W. K., and Terzic, A. Adenosine prevents K⁺-induced Ca²⁺ loading: Insight into cardioprotection during cardioplegia. Ann. Thorac. Surg. 1998; 65:586-591.
167. Lasley R D, and Mentzer, R. M. Protective effects of adenosine in the reversibly injured heart. Ann of Thorac Surg. 1995; 60:843-846.
168. Hollmann M W, Difazio, C. A., and Durieux, M. E. Ca-signaling G-protein-coupled receptors: a new site of local anesthetic action? Reg Anesth Pain Med. 2001; 26:565-571.
169. Masuda T, Dobson, G. P., and Veech, R. L. The Gibbs-Donnan near-equilbrium system of heart. J. Biol. Chem. 1990; 265:20321-20334.
170. Dobson G P, Veech, R. L., Hoeger, U., and Passonneau, J. V. Enzymatic determination of total CO₂ in freeze-clamped animal tissues and plasma. Anal. Biochem. 1991; 195:232-237.
171. Hearse D J, Stewart, D. A., Braimbridge, M. V. Myocardial protection during ischaemic cardiac arrest. Possible deleterious effects of glucose and mannitol in coronary infusates. J. Thorac Cardiovasc Surg. 1978; 76:16-23.
172. Neely J R, and Rovetto, M. J., ed. Techniques for perfusing isolated rat hearts. London. New York: Academic Press; 1975.
173. Dobson G P, and Cieslar, J. H. Intracellular, interstitial and plasma spaces in the rat myocardium in vivo. J. Mol. Cell Cardiol. 1997; 29:3357-3363.
174. Altman P L, and Dittmer, D. S., ed. Respiration and Circulation. Bethesda, Md.: Federation of American Societies for Experimental Biology; 1971.
175. Christoforides C, Laasberg, L. H., and Hedley-Whyte, J. Effect of temperature on solubility Of O₂ in human plasma. J. Appl. Physiol. 1969; 26:56-60.
176. Grover G J, and Garlid, K. D. ATP-Sensitive potassium channels: a review of their cardioprotective pharmacology. J Mol Cell Cardiol. 2000; 32:677-695.
177. Ou R, Gavin, J. B., Esmore, D. S., and Rosenfeldt, F. L. Increased temperature reduces the protective effect of university of Wisconsin solution in the heart. Ann. Thorac. Surg. 1999; 68:1628-1634.
178. Mankad P S, Chester, A. H., and Yacoub, M. H. Role of potassium concentration in cardioplegic solutions in mediating endothelial damage. Ann. Thorac. Surg. 1991; 51:89-93.
179. Cohen N M, Wise, R. M., Wechsler, A. S., and Damiano, R. J. Elective cardiac arrest with a hyperpolarising adenosine triphosphate-sensitive potassium channel opener: A novel form of myocardial protection? J. Thorac. Cardiovasc. Surg. 1993; 106:317-328.
180. Jayawant A M, Lawton, J. S., Hsia, P. W., and Damiano, R. J. Hyperpolarised cardioplegic arrest with Nicorandil. Circulation (9 Suppl. 2). 1997; 96:11240-246.
181. Lorenzen A, Guerra, L., Vogt, H., and Schwabe, U. Interaction of full and partial agonists of the A1 adenosine receptor with receptor/G protein complexes in rat brain membranes. Mol. Pharmacol. 1996; 49:915-926.

1-25. (canceled)

26. A method for reducing electrical disturbance of a cell's resting membrane potential comprising administering to the cell an effective amount of a composition comprising an effective amount of (i) a local anaesthetic; and (ii) at least one of a potassium channel opener, an adenosine receptor agonist, an anti-adrenergic, a calcium antagonist, an opioid, an NO donor and a sodium hydrogen exchange inhibitor, said composition further comprising a physiological potassium concentration.

27. A method for reducing damage to a cell, tissue or organ following ischaemia comprising administering to the cell, tissue or organ an effective amount of a composition comprising an effective amount of (i) a local anesthetic; and (ii) at least one of a potassium channel opener, an adenosine receptor agonist, an anti-adrenergic, a calcium antagonist, an opioid, an NO donor and a sodium hydrogen exchange inhibitor, said composition further comprising a physiological potassium concentration.

28. A method for preconditioning a cell or tissue during ischaemia or reperfusion comprising administering an effective amount of a composition comprising an effective amount of (i) a local anesthetic; and (ii) at least one of a potassium channel opener, an adenosine receptor agonist, an anti-adrenergic, a calcium antagonist, an opioid, an NO donor and a sodium hydrogen exchange inhibitor, said composition further comprising a physiological potassium concentration.

29. A method for reducing damage to a cell, organ or tissue before, during and following a surgical or clinical intervention comprising administering to the cell, organ or tissue an effective amount of a composition comprising an effective amount of (i) a local anaesthetic; and (ii) at least one of a potassium channel opener, an adenosine receptor agonist, an anti-adrenergic, a calcium antagonist, an opioid, an NO donor and a sodium hydrogen exchange inhibitor, said composition further comprising a physiological potassium concentration.

30. A method according to claim 27 wherein the anti-adrenergic is selected from beta-blockers and alpha(1)-adrenoceptor-antagonists.

31. A method according to claim 27 wherein the opioid is selected from enkephalins, endorphins and dynorphins.

32. A method according to claim 27 wherein the opioid is a delta opioid receptor agonist.

33. A method according to claim 27 wherein the calcium antagonist is selected from Amlodipine, nifedipine, nicardipine, nimodipine, nisoldipine, lercanidipine, telodipine, angizem, altiazem, bepridil, amlodipine, felodipine, mibefradil, isradipine, cavero, Bay K 8644(L-type) (1,4-dihydro-26-dimethyl-5-nitro-[2(trifluoromethyl)pheny1]-3-pyridine carboxylic acid (methyl ester)), calciseptine (L-type), omega-conotoxin GVIA (N-type), omega-conotoxin MVIIC (Q-type), cyproheptadine HCI, dantrolene sodium, diltiazem HCI (L-type), filodipine, flunarizine HCI (Ca2+/Na+), fluspirilene (L-type), HA-10772HCI(1-(5 isoquinolinyl sulphonyl) homo piperazine.HCI), isradipine, loperamide HCI, manoalide, niguldipine HCI (L-type), nitrendipine (L-type), pimozide (L- and T-type), ruthenium red, ryanodine (SR channels), taicatoxin, verapamil HCI (L-type), Azelnidipine (L-type) methoxy-verapamil HCI (L-type), YS-035HCI (L-type)N[2(3,4-dimethoxyphenyl)ethyl]-3,4-dimethoxy N-methyl benzene ethaneamine HCI) and calcium antagonists with AV blocking actions.

34. A method according to claim 27 wherein the NO donor is either nitric-oxide synthase independent or nitric-oxide synthase dependent.

35. A method according to claim 27 wherein the sodium hydrogen exchange inhibitor is selected from the group consisting of amiloride, cariporide, eniporide, triamterene and EMD 84021, EMD 94309, EMD 96785, and T-162559.

36. A method according to claim 27 wherein the cell is a myocyte, endothelial cell, smooth-muscle cell, neutrophil, platelet and other inflammatory cells, or the tissue is heart tissue or vasculature, or the organ is a heart.

37. A method according to claim 27 wherein the composition further comprises one or more of an antioxidant, ionic magnesium, an impermeant and a metabolic substrate.

38. A method according to claim 27 wherein the composition has been oxygenated.

39. A method according to claim 27 comprising administering the composition as part of a medicament including the composition and a pharmaceutically acceptable carrier, diluent, adjuvant, and/or excipient.

40. A method according to claim 39 wherein the medicament has concentrations of one or more of sodium, calcium and chloride lower than physiological concentrations.

41. A method according to claim 27 wherein the composition is at a temperature of profound hypothermia (0 to 4 degrees Celsius), moderate hypothermia (5 to 20 degrees Celsius), mild hypothermia (20 to 32 degrees Celsius) or normothermia (32 to 38 degrees Celsius).

42. A method according to claim 27 wherein the components of the medicament or composition are combined before administration or when the components are administered substantially simultaneously or co-administered.

43. A method according to claim 30, wherein the alpha(1)-adrenoceptor-antagonist is prazosin.

44. A method according to claim 30, wherein the beta-blocker is selected from esmolol, atenolol, metoprolol and propranolol.

45. A method according to claim 31, wherein the opioid is an enkephalin which targets delta, alpha and/or mu receptors.

46. A method according to claim 31, wherein the opioid is DPDPE.

47. A method according to claim 33, wherein the calcium antagonist is verapamil.

48. A method according to claim 34, wherein the nitric-oxide synthase independent NO donor is selected from nitroprusside, nitro-glycerine, flurbiprofen or its NO-donating derivative, HCT1026 (2-fluoro-a-methyl[1,1¹-biphenyl]-4-acetic acid and 4-(nitrooxy)butyl ester.

49. A method according to claim 30, wherein the nitric-oxide synthase dependent No-donor is regulator calcium calmodulin and L-arginine.
